(19) United States
(12) Patent Application Publication
(10) Pub. No.: US 2021/0247403 A1
(43) Pub. Date: Aug. 12, 2021

(54) MARKERS OF IMMUNE WELLNESS AND METHODS OF USE THEREOF

(71) Applicant: HealthTell Inc., San Ramon, CA (US)

(72) Inventors: Aaron ARVEY, San Ramon, CA (US); Gang AN, San Ramon, CA (US); Ted TARASOW, San Ramon, CA (US); Bill COLSTON, San Ramon, CA (US)

(21) Appl. No.: 17/050,366

(22) PCT Filed: Apr. 23, 2019
(86) PCT No.: PCT/US2019/028791

§ 371 Date: Oct. 23, 2020

Related U.S. Application Data
(60) Provisional application No. 62/662,131, filed on Apr. 24, 2018.

(51) Int. Cl. G01N 33/68; G16H 50/20; G16H 50/30

(52) U.S. Cl. CPC G01N 33/6854; G01N 2800/60; G16H 50/30; G16H 50/20

(57) ABSTRACT

Provided herein are methods of measuring immune health in a subject. In some embodiments, methods herein comprise, obtaining an immunological measurement from a biological sample from the subject, wherein the immunological measurement comprises antibody-peptide binding. Also provided are computer-implemented methods of predicting immune health, including computer-implemented machine learning algorithms useful in such predictions.

### CROSS-REFERENCE

This application claims the benefit of U.S. Provisional Application No. 62/662,131 filed on Apr. 24, 2018, entitled “Markers Of Immune Wellness And Methods Of Use Thereof,” which is incorporated herein by reference in its entirety. All publications, patents, and patent applications mentioned in this specification are herein incorporated by reference to the same extent as if each individual publication, patent, or patent application was specifically and individually indicated to be incorporated by reference.

### BACKGROUND OF THE INVENTION

Immune function has a primary role of combating foreign agents, such as pathogens and the like. In addition, the immune system carries out an important function in eradicating cancer and precancerous lesions. Malfunction of the immune system can lead to immune deficiencies or autoimmune disorders. Despite the prominent role of the immune system in various disorders, there is generally a lack of methods to evaluate and assess an individual's immune system.

### SUMMARY OF THE INVENTION

Provided herein are methods of measuring immune health in a subject. In some embodiments, methods herein comprise, obtaining an immunological measurement from a biological sample from the subject, wherein the immunological measurement comprises antibody-peptide binding. In some embodiments, the immunological measurement further comprises measurement of one or more of the group consisting of: an immune protein sequence, a cytokine level, a metabolite level, and a blood cell count. In some embodiments, the cytokine is selected from TNFα, GM-CSF, MCP-1 (CCL2), MCP-3, IFNα, IFNγ, IL1β, IL2, IL4, IL5, IL6, IL7, IL8, IL10, IL12, IL13, IL17, IL18, IL21, CRP, EGFR, IP10 (CXCL10), Eotaxin (CCL11), MIG, AGP, sTNF-RI, sTNF-RII, sL2RA, sIL1RA, sIL1RII, sIL6R, CD40L, IL18BP, EGF, VEGF, resistin, leptin, adiponectin, alpha-1-antitrypsin, and free fatty acids. In some embodiments, the cytokine is selected from CD40L, EGF, Eotaxin (CCL11), GM-CSF, IFNα, IFNγ, IL-1β, sIL-RA, sIL-2R, IL-6, IP-10 (CXCL10), MCP-1 (CCL2), TNFα, sTNF-RI, and sTNF-RII. In some embodiments, the cytokine is selected from Eotaxin (CCL11), sIL-1RA, sIL-2R, sTNF-RI, IP10 (CXCL10), TNFα, IFNα, IFNγ, IL6, sTNF-RII, and IL-1β. In some embodiments, the cytokine level is measured in a biological fluid. In some embodiments, the biological fluid is selected from the group consisting of serum, whole blood, dried blood, plasma, saliva, and a combination thereof. In some embodiments, the cytokine level is measured in a cytokine assay selected from the group consisting of a bead assay, an aptamer assay, an ELISA assay, and an ELISPOT assay.

In some instances, antibody-peptide binding is measured using an immobilized peptide capture assay, including but not limited to a peptide array, a microtiter plate assay or a bead assay. In some embodiments, antibody-peptide binding is measured using a peptide array binding assay, wherein the peptide array binding assay comprises: (a) contacting a sample from the subject to a peptide array comprising a plurality of different peptides on distinct features of the array; (b) detecting the binding of antibodies present in the sample to a set of peptides on the peptide array to obtain a pattern of binding signals, wherein the pattern comprises binding signals each associated with a distinct peptide on the array; and (c) comparing the pattern of binding signals in the sample to the pattern of binding signals obtained in reference samples, wherein the binding signals obtained from the binding of the sample correspond to a same set of peptides predictive of immune health identified in a plurality of healthy reference subjects, thereby determining the immune health of the subject. In some embodiments, the sample is a biological fluid. In some embodiments, the biological fluid is selected from the group consisting of whole blood, serum, plasma, saliva, and a combination thereof. In some embodiments, blood is dried blood. In some embodiments, the peptide array is a peptide microarray. In some embodiments, the peptide array comprises at least about 10,000 distinct peptides. In some embodiments, the peptide array comprises at least about 3,000,000 distinct peptides. In some embodiments, the peptide array comprises peptides having 20 or fewer amino acids. In some embodiments, the peptide array comprises peptides having at least 20 amino acids. In some embodiments, the peptide array comprises peptides comprising natural amino acids. In some embodiments, the peptide array comprises peptides comprising unnatural amino acids. In some embodiments, the peptide array comprises a plurality of peptides characterized by at least one serine motif, threonine motif, serine-threonine motif, or any combination thereof. In some embodiments, the serine motif comprises S, SS, SSS, or SSSS. In some embodiments, the threonine motif comprises T or TT. In some embodiments, the serine-threonine motif comprises TS or ST. In some embodiments, each of the at least one serine motif, threonine motif, or serine-threonine motif is positioned no more than 1, 2, 3, 4, 5 or 6 amino acids from the N-terminus. In some embodiments, the plurality of peptides are acetylated. In some embodiments, a machine learning algorithm generates a prediction of immune health based on the immunological measurement. In some embodiments, the machine learning algorithm comprises a panel of peptide features comprising the plurality of peptides characterized by at least one serine motif, threonine motif, serine-threonine motif, or any combination thereof. In some embodiments, the plurality of peptides make up at least 10%, 20%, 30%, 40%, 50%, 60%, 70%, 80%, 90%, or 95% of the panel of peptide features. In some embodiments, the plurality of peptides comprises at least 50, 100, 150, 200, 250, 300, or 350 peptides. In some embodiments, at least 10%, 20%, 30%, 40%, 50%, 60%, 70%, 80%, 90%, or 95% of the plurality of peptides are statistically correlated with age. In some embodiments, at least 10%, 20%, 30%, 40%, 50%, 60%, 70%, 80%, 90%, or 95% the plurality of peptides that are statistically correlated with age have an N-terminal di-serine (SS) motif. In some embodiments, the peptide array comprises peptides having a sequence comprising EX₁(X₂)ₙ (SEQ ID NO: 1), wherein X₁ comprises an amino acid selected from A, S, R, Y, and V, X₂ comprises any amino acid. In some embodiments, n is between 3 and 30. In some embodiments, antibody-peptide binding to a peptide having a sequence of SEQ ID NO: 1 is associated with body mass index (BMI). In some embodiments, the peptide array comprises peptides having a sequence comprising SS(X)ₙ (SEQ ID NO: 2), wherein X comprises any amino acid. In some embodiments, n is between 3 and 30. In some embodiments, antibody-peptide binding to a peptide having a sequence of SEQ ID NO: 2 is associated with chronological age.

In some embodiments, the plurality of healthy reference subjects are subjects not having an immune altering condition. In some embodiments, the immune altering condition is selected from an autoimmune disease, an inflammatory disease, an immunodeficiency disease, and a cancer. In some embodiments, the set of peptides predictive of immune health are identified by a method comprising: (i) providing a same peptide array and contacting a plurality of reference samples from a plurality of reference subjects to the peptide array; (ii) detecting the binding of antibodies present in each of the reference samples to the peptides on the array to obtain a pattern of binding signals for each of the reference samples, wherein each pattern of binding signals corresponds to one of a range of known measurements of at least one marker of immune health; (iii) measuring the binding signal associated with each peptide in each of the pattern of binding signals obtained for each of the reference samples; (iii) determining the correlation of the binding signal for each of the peptides in the plurality of reference samples to the range of measurements of the at least one known marker of immune health; and (iv) identifying a set of peptides having a combination of binding signals that correlates to the at least one marker of immune health, thereby identifying the set of peptides predictive of immune health. In some embodiments, range of known measurements is selected for chronological age and body mass index. In some embodiments, step (iv) comprises using a statistical model selected from Elastic Net regression, SVM, and neural networks. In some embodiments, the at least one marker of immune health is selected from chronological age, body mass index, at least one cytokine, and a combination thereof. In some embodiments, the at least one marker of immune health further comprises one or more of the group consisting of an immune protein sequence, a cytokine level, a metabolite level, and a blood cell count.

In some embodiments, the immune health of the subject corresponds to an immunological measurement that is less than, equal to, or greater than the same immunological measurement obtained in the healthy reference subjects having a chronological age corresponding to the immune age of the subject. In some embodiments, the immune health corresponds to a chronological age that is greater than, equal to, or less than the chronological age of the subject, thereby determining that the immune health of the subject is greater than, equal to, or less than the immune age of healthy reference subjects. In some embodiments, the immune health corresponds to a BMI that is greater than, equal to, or less than the BMI of the subject, thereby determining that the immune health of the subject is greater than, equal to, or less than the immune health of healthy reference subjects. In some embodiments, the immune health corresponds to a combination of chronological age and BMI that is greater than, equal to, or less than the chronological age of the subject, thereby determining that the immune health of the subject is greater than, equal to, or less than the immune health of healthy reference subjects. In some embodiments, the marker is chronological age, and wherein the set of peptides predictive of immune health comprise at least one of the sequence motifs provided in Table 1. In some embodiments, the marker is chronological age, and wherein the set of peptides predictive of immune health comprise at least one of the following sequence motifs: S, SS, SSS, SSSS, ST, TS, TT, or TTT. In some embodiments, the marker is BMI, and wherein the set of peptides predictive of immune health comprise at least one of the sequence motifs provided in Table 2. In some embodiments, the marker is BMI, and wherein the set of peptides predictive of immune health comprise at least one of the following sequence motifs: S, SS, SSS, SSSS, ST, TS, TT, or TTT. In some embodiments, the immune protein is selected from the group consisting of an immunoglobulin and a T cell receptor. In some embodiments, the immune protein sequence is determined by sequencing a nucleic acid encoding the immune protein. In some embodiments, the metabolite is selected from a fatty acid, an amino acid, a sugar, an enzyme substrate, and combinations thereof. In some embodiments, the blood cell is selected from one or more of an erythrocyte, a leukocyte, a neutrophil, an eosinophil, a basophil, a lymphocyte, a T cell, a CD4+ T cell, a CD8+ T cell, a regulatory T cell, a γδ T cell, a natural killer cell, a natural killer T cell, a monocyte, a macrophage, and a platelet. In some embodiments, the method comprises providing a recommendation for the subject based on the measured immune health. In some embodiments, the recommendation comprises providing treatment to the subject, stopping treatment of the subject, adopting a lifestyle change, or obtaining testing for one or more immune-related diseases, disorders, or conditions. In some embodiments, the method comprises providing a therapy or treatment to the subject based on the measured immune health. In some embodiments, the method comprises providing further testing to the subject based on the measured immune health. In some embodiments, the further testing comprises genetic testing, metabolite testing, serum protein testing, blood cell count testing, immunoglobulin testing, or any combination thereof.

Also provided herein are computer-implemented methods of predicting an immune health of a subject. In some embodiments, computer-implement methods of predicting immune health comprise: ingesting, by a computer, results of an immunological measurement from a biological sample from the subject, wherein the immunological measurement comprises antibody-peptide binding; and applying, by the computer, a machine learning algorithm to the results of the immunological measurement to predict the immune health of the subject. In some embodiments, methods herein further comprise performing, by the computer, feature selection. In some embodiments, the feature selection is performed by t-test, correlation, principal component analysis (PCA), or a combination thereof. In some embodiments, the machine learning algorithm is implemented as: a linear classifier, a neural network, a support vector machine (SVM), an adaptively boosted classifier (AdaBoost), decision tree learning, or a combination thereof. In some embodiments, the machine learning algorithm is implemented as a linear classifier, and wherein a linear model is learned by elastic net. In some embodiments, the machine learning algorithm is implemented as ridge regression, lasso regression, regression trees, forward stepwise regression, backward elimination, support vector regression, or a combination thereof. In some embodiments, methods herein further comprise comparing, by the computer, a proxy measure of the immune health of the subject to the predicted immune health of the subject to determine a residual score. In some embodiments, the residual score is an indicator of immune health or immunosenescence. In some embodiments, the proxy measure of the immune health of the subject comprises: chronological age, body mass index (BMI), immune disease or immune disease state, response to treatment in autoimmune disease, response to treatment in immunotherapy, erythrocyte sedimentation rate, antinuclear autoantibodies, rheumatoid factor, fibrinogen, T cell TCR diversity, B cell immunoglobulin diversity, quantification of lymphocytes, quantification of myeloid cells, endogenous steroids, quantification of complement, or a combination thereof. In some embodiments, the proxy measure of the immune health of the subject comprises a combination of chronological age and body mass index (BMI). In some embodiments, the predicted immune health is expressed as an immune age. In some embodiments, methods herein further comprise ingesting survey data pertaining to the current or past health of the subject, and wherein the machine learning algorithm is further applied to the survey data. In some embodiments, methods herein further comprise generating, by the computer, a report. In some embodiments, the report is implemented as a mobile application or a web application. In some embodiments, the immunological measurement further comprises one or more of the group consisting of: antibody-peptide binding, an immune protein sequence, a cytokine level, a metabolite level, and a blood cell count. In some embodiments, the cytokine is selected from TNFα, GM-CSF, MCP-1 (CCL2), MCP-3, IFNα, IFNγ, IL1β, IL2, IL4, IL5, IL6, IL7, IL8, IL10, IL12, IL13, IL17, IL18, IL21, CRP, EGFR, IP10 (CXCL10), Eotaxin (CCL11), MIG, AGP, sTNF-RI, sTNF-RII, sIL2RA, sIL1RA, sIL1RII, sIL6R, CD40L, IL18BP, EGF, VEGF, resistin, leptin, adiponectin, alpha-1-antitrypsin, and free fatty acids. In some embodiments, the cytokine is selected from CD40L, EGF, Eotaxin (CCL11), GM-CSF, IFNα, IFNγ, IL-1β, sIL-1RA, sIL-2R, IL-6, IP-10 (CXCL10), MCP-1 (CCL2), TNFα, sTNF-RI, sTNF-RII. In some embodiments, the cytokine is selected from Eotaxin (CCL11), sIL-1RA, sIL-2R, sTNF-RI, IP10 (CXCL10), TNFα, IFNα, TFNγ, IL6, sTNF-RII, and IL-1β. In some embodiments, cytokine level is measured in a biological fluid. In some embodiments, the biological fluid is selected from the group consisting of serum, whole blood, dried blood, plasma, saliva, and a combination thereof. In some embodiments, the cytokine level is measured in a cytokine assay selected from the group consisting of a bead assay, an aptamer assay, an ELISA assay, and an ELISPOT assay. In some embodiments, antibody-peptide binding is measured in a peptide array binding assay, wherein the peptide array binding assay comprises: (a) contacting a sample from the subject to a peptide array comprising a plurality of different peptides on distinct features of the array; (b) detecting the binding of antibodies present in the sample to a set of peptides on the peptide array to obtain a pattern of binding signals, wherein the pattern comprises binding signals each associated with a distinct peptide on the array; and (c) comparing the pattern of binding signals in the sample to the pattern of binding signals obtained in reference samples, wherein the binding signals obtained from the binding of the sample correspond to a same set of peptides predictive of immune health identified in a plurality of healthy reference subjects, thereby determining the immune health of the subject. In some embodiments, the sample is a biological fluid. In some embodiments, the biological fluid is selected from the group consisting of blood, serum, plasma, saliva, and a combination thereof. In some embodiments, blood is dried blood. In some embodiments, the peptide array is a peptide microarray. In some embodiments, the peptide array comprises about 10,000 distinct peptides. In some embodiments, the peptide array comprises about 3,000,000 distinct peptides. In some embodiments, the peptide array comprises peptides having 20 or fewer amino acids. In some embodiments, the peptide array comprises peptides having at least 20 amino acids. In some embodiments, the peptide array comprises peptides comprising natural amino acids. In some embodiments, the peptide array comprises peptides comprising unnatural amino acids. In some embodiments, the peptide array comprises a plurality of peptides characterized by at least one serine motif, threonine motif, serine-threonine motif, or any combination thereof. In some embodiments, the serine motif comprises S, SS, SSS, or SSSS. In some embodiments, the threonine motif comprises T or TT. In some embodiments, the serine-threonine motif comprises TS or ST. In some embodiments, each of the at least one serine motif, threonine motif, or serine-threonine motif is positioned no more than 1, 2, 3, 4, 5 or 6 amino acids from the N-terminus. In some embodiments, the plurality of peptides are acetylated. In some embodiments, the machine learning algorithm comprises a panel of peptide features comprising the plurality of peptides characterized by at least one serine motif, threonine motif, serine-threonine motif, or any combination thereof. In some embodiments, the plurality of peptides make up at least 10%, 20%, 30%, 40%, 50%, 60%, 70%, 80%, 90%, or 95% of the panel of peptide features. In some embodiments, the plurality of peptides comprises at least 50, 100, 150, 200, 250, 300, or 350 peptides. In some embodiments, at least 10%, 20%, 30%, 40%, 50%, 60%, 70%, 80%, 90%, or 95% of the plurality of peptides are statistically correlated with age. In some embodiments, at least 10%, 20%, 30%, 40%, 50%, 60%, 70%, 80%, 90%, or 95% the plurality of peptides that are statistically correlated with age have an N-terminal di-serine (SS) motif. In some embodiments, the peptide array comprises peptides having a sequence comprising EX₁(X₂)ₙ (SEQ ID NO: 1), wherein X₁ comprises an amino acid selected from A, S, R, Y, and V, X₂ comprises any amino acid. In some embodiments, n is between 3 and 30. In some embodiments, antibody-peptide binding to a peptide having a sequence of SEQ ID NO: 1 is associated with body mass index (BMI). In some embodiments, the peptide array comprises peptides having a sequence comprising SS(X)ₙ (SEQ ID NO: 2), wherein X comprises any amino acid. In some embodiments, n is between 3 and 30. In some embodiments, antibody-peptide binding to a peptide having a sequence of SEQ ID NO: 2 is associated with chronological age. In some embodiments, the plurality of healthy reference subjects are subjects not having an immune altering condition. In some embodiments, the immune altering condition is selected from an autoimmune disease, an inflammatory disease, an immunodeficiency disease, and a cancer. In some embodiments, the set of peptides predictive of immune health are identified by a method comprising: (i) providing a same peptide array and contacting a plurality of reference samples from a plurality of reference subjects to the peptide array; (ii) detecting the binding of antibodies present in each of the reference samples to the peptides on the array to obtain a pattern of binding signals for each of the reference samples, wherein each pattern of binding signals corresponds to one of a range of known measurements of at least one marker of immune health; (iii) measuring the binding signal associated with each peptide in each of the pattern of binding signals obtained for each of the reference samples; (iii) determining the correlation of the binding signal for each of the peptides in the plurality of reference samples to the range of measurements of the at least one known marker; and (iv) identifying a set of peptides having a combination of binding signals that correlates to the at least one marker of immune health, thereby identifying the set of peptides predictive of immune health. In some embodiments, range of known measurements is selected for chronological age and body mass index. In some embodiments, step (iv) comprises using a statistical model selected from Elastic Net regression, SVM, and neural networks. In some embodiments, the at least one marker of immune health is selected from chronological age, body mass index, at least one cytokine, and a combination thereof. In some embodiments, the at least one marker of immune health further comprises one or more of the group consisting of an immune protein sequence, a cytokine level, a metabolite level, and a blood cell count. In some embodiments, the immune health of the subject corresponds to an immunological measurement that is less than, equal to, or greater than the same immunological measurement obtained in the healthy reference subjects having a chronological age corresponding to the immune health of the subject. In some embodiments, the immune health corresponds to a chronological age that is greater than, equal to, or less than the chronological age of the subject, thereby determining that the immune health of the subject is greater than, equal to, or less than the immune age of healthy reference subjects. In some embodiments, the immune health corresponds to a BMI that is greater than, equal to, or less than the BMI of the subject, thereby determining that the immune health of the subject is greater than, equal to, or less than the immune health of healthy reference subjects. In some embodiments, the immune health corresponds to a combination of chronological age and BMI that is greater than, equal to, or less than the chronological age of the subject, thereby determining that the immune health of the subject is greater than, equal to, or less than the immune health of healthy reference subjects. In some embodiments, the marker is chronological age, and wherein the set of peptides predictive of immune health comprise at least one of the sequence motifs provided in Table 1. In some embodiments, the marker is chronological age, and wherein the set of peptides predictive of immune health comprise at least one of the following sequence motifs: S, SS, SSS, SSSS, ST, TS, TT, or TTT. In some embodiments, the marker is BMI, and wherein the set of peptides predictive of immune health comprise at least one of the sequence motifs provided in Table 2. In some embodiments, the marker is BMI, and wherein the set of peptides predictive of immune health comprise at least one of the following sequence motifs: S, SS, SSS, SSSS, ST, TS, TT, or TTT. In some embodiments, the method comprises providing a recommendation for the subject based on the measured immune health. In some embodiments, the recommendation comprises providing treatment to the subject, stopping treatment of the subject, adopting a lifestyle change, or obtaining testing for one or more immune-related diseases, disorders, or conditions. In some embodiments, the method comprises providing a therapy or treatment to the subject based on the measured immune health. In some embodiments, the method comprises providing further testing to the subject based on the measured immune health. In some embodiments, the further testing comprises genetic testing, metabolite testing, serum protein testing, blood cell count testing, immunoglobulin testing, or any combination thereof. In another aspect, disclosed herein is a computer system comprising a processor and non-transitory computer readable storage medium encoded with a computer program that causes the processor to perform any of the methods of the present disclosure, including at least the computer-implemented methods of this paragraph.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application file contains at least one drawing executed in color. Copies of this patent application with color drawing(s) will be provided by the Office upon request and payment of the necessary fee. An understanding of the features and advantages of the present invention will be obtained by reference to the following detailed description that sets forth illustrative embodiments, in which the principles of the invention are utilized, and the accompanying drawings of which:

FIG. 1 shows schematic of cohort recruitment for training regression model. Samples were obtained based on their binning by age group, sex, and BMI. Reference is made to Example 1.

FIG. 2 shows a graph plotting the correlation between the Immune Index (ImmunoSignature prediction) with chronological age (top panel) and BMI (bottom panel) using linear models learned by elastic net. Reference is made to Example 1.

FIG. 3 shows a graph of the correlation between the ImmunoSignature prediction (e.g., immune health prediction) with chronological age determined in separate models developed using samples from different training sets. Reference is made to Example 1.

FIG. 4A-4G shows graphs plotting the relationship of cytokine levels with chronological age and with BMI. FIG. 4A plots the relationship between IP10 and age (r=0.14). FIG. 4B plots the relationship between Eotaxin and age (r=0.27). FIG. 4C plots the relationship between sCD40L and age (r=0.13). FIG. 4D plots the relationship between sIL2Ra and age (r=0.16).

FIG. 4E plots the relationship between sTNFR1 and age (r=0.18). FIG. 4F plots the relationship between sIL1Ra and BMI (r=0.38). FIG. 4G plots the relationship between CRP and BMI (r=0.36). Reference is made to Example 2.

FIG. 5 shows graphs showing on the y-axis, the relationship between immunosignatures (i.e. peptide array signals) shown in row A, cytokines (row (B)), and combinations of immunosignatures and cytokines (rows (C) and (D)) and on the x-axis, a combination function of chronological age and BMI (column (i)), chronological age (column (ii)) and BMI (column (iii)), as proxies for immune health. Row (C) trained on example matrix where peptide array and cytokine data were concatenated, whereas row (D) trained on matrix where only score derived from peptide array data was concatenated to cytokine data. Cytokine data was transformed by log 10(x+1) to make linear regression variance more homoscedastic. In this context, “concatenation” refers to combining two matrices (organized as donors as rows and measurements in columns) by adjoining column-wise after matching rows by donor. Reference is made to Example 3.

FIG. 6 shows a graph of chronological age-based ImmunoSignature data (y-axis) and ImmunoSignature data based on cytokine levels (x-axis) indicating an association between these parameters. Reference is made to Example 3.

FIG. 7 shows a graph of peptide-array binding data of secondary antibody binding in the absence of serum as a measure of background binding, (y-axis), and the log ratio of the age difference between older i.e. >65 years and younger <40 years samples (x-axis). Reference is made to Example 5.

FIG. 8 shows graphs of the log 10 ratio of donors >65 yo and donors <40 yo (x-axis; each dot is single probe) versus the log 10 fold change of probes in response to serum spiked with triglycerides, rheumatoid factor (RF), conjugated bilirubin, HAMA (human anti-mouse antibody), hemoglobin, and unconjugated bilirubin (y-axis). Reference is made to Example 5.

FIG. 9 shows graphs of the log 10 ratio of donors with BMI <24 and BMI >33 (x-axis; each dot is single probe) versus the log 10 fold change of probes in response to level of triglycerides, rheumatoid factor (RF), conjugated bilirubin, HAMA (human anti-mouse antibody), hemoglobin, and unconjugated bilirubin. Reference is made to Example 5.

FIG. 10 shows an exemplary process by which models or classifiers are trained and selected. Reference is made to Example 6.

FIG. 11 shows a graph correlating the residuals across multiple training sets to provide a summary of the bias-variance tradeoff. The x-axis shows a model trained on San Francisco (SF) samples, whereas y-axis shows model trained on samples where Other-where in California (OC). Both x- and y-axes show the value of prediction residual when applying the learned model on a validation set of 1074 samples. Reference is made to Example 7.

FIG. 12 shows a graph plotting the immune index residual 80% interval against the immune index residual (mean). Reference is made to Example 7.

FIG. 13 shows a graph plotting the immune index over time for individual donors. Time points on x-axis are days 0, 2, 4, 7, and 28. Reference is made to Example 7.

FIGS. 14A-C show data demonstrating the phenotypic association of the immune index with SLE and SLEDAI. Reference is made to Example 7. FIG. 14A shows the immune index residual values (y-axis) of samples obtained from reference healthy subjects and groups of subjects with fibromyalgia, osteoarthritis (OA), psoriatic arthritis, rheumatoid arthritis (RA), systemic lupus erythematous (SLE), and Sjogren's syndrome (SS) in order from left to right.

FIG. 14B shows the correlation of Immune Index values with chronological age in the healthy subjects shown in FIG. 14A. FIG. 14C shows the correlation of Immune Index residual with the SELENA-SLEDAI score for subjects with SLE.

FIG. 15 shows a concept plot of a hypothetical dataset. Each point is a single donor and the y-axis shows an idealized immunosignature score for immune index, whereas the x-axis represents a function of Age and BMI i.e. F(Age, BMI).

FIG. 16A-16C show graphs plotting parameter selection criteria (accuracy, residual correlation, mean squared difference, and number of features in model) against elastic net model parameters (alpha (a) and lambda (k) values). The model was optimized for both accuracy and lower variance by finding parameters that yield high accuracy (Pearson's correlation) and lower variance (highly correlated residuals and lower mean squared differences) when training on independent training sets. All optimization criteria were evaluated on a hold set validation set of 1074 samples.

FIG. 17 shows an exemplary report summarizing the results of an immune index score.

FIG. 18 shows an exemplary digital processing device configured to carry out the methods described herein.

FIG. 19A-E show an exemplary optimization of peptide microarray. FIG. 19A depicts the clustering of probes in grid pattern affixed a wafer chip. FIG. 19B and FIG. 19C illustrate one of the optimization steps where the foreground (FG) intensity was measured and the coefficient of variance (CV) reported to ensure consistency across multiple wafers (40010, 40011, 40025, 40027, 40037, 40038, 40039, and 40040). FIG. 19D and FIG. 19E illustrate that the wafer-to-wafer (FIG. 19D) and experiment-to-experiment (FIG. 19E) residuals or results were correlated.

FIG. 20A-20C summarizes the improvement of the data output and analysis based on the optimization of peptide microarray. Accuracy, residual correlation, mean squared difference, and number of features in models were all improved with the optimization.

FIG. 21A-B show the composition of the recruited cohort (FIG. 21A), and the statistical analysis (FIG. 21C) showing the two lines with lower bound for confidence intervals assuming point estimates of r=0.7 and r=0.6. The conservative estimate of r=0.6 yielded a 99%-confidence interval that excluded r<0.5 when N >˜400. Therefore, at least 400 serum donors were needed to obtain a robust estimate of Pearson's correlation coefficient that would pass the threshold of r >0.5. FIG. 21C demonstrates the power analysis for detecting impact of factor (ethnicity, left) and factor interacting with covariate (Age, right). Number of samples was number of samples required per ethnicity that was to be pair-wise compared. If there were 4 ethnicities, only 2 ethnicities at a time were compared against one another, each composed of N samples. Power was shown for significance level of α=0.05 (dashed line). Power was based on simulation of nominal significance and did not include multiple hypothesis correction.

FIG. 22A-C illustrate age-associated antibody binding and affinity for serine residues in significant subset of older individuals. FIG. 22A shows that if a donor had high intensity binding to an SS-feature, it was likely that the donor's serum would also bind highly to other probes that began with “SS”. FIG. 22B shows that the distinct pattern of the imaging mass spectrometry (IMS) was due to more prevalent SS probes in older donors. FIG. 22C shows that high intensity fold change probes were SS probes, which were more abundant in donors who were older than 60 years old than young donors who were younger than 42 years old. FIG. 22D shows 20 of the top 100 most age-associated probes which are highly correlated with one another across 1675 donors.

FIG. 23A-D show the affinity of age-associated antibody binding for serine residues in older individuals. FIG. 23A is an exemplary workflow of the peptide array assy. FIG. 23B demonstrates that a set of peptide features were significantly associated with older vs younger serum donors, and FIG. 23C further shows that the antibody-affinity patterns were associated with age. FIG. 23D is an exemplary list of SS peptides identified in the IMS for predicting immune age. The list comprises highest ranking SS-peptides, which have the largest fold changes in older, as opposed to younger, donors.

FIG. 24A illustrates the binding affinity for the probes comprising serine residues. Serine and threonine are structurally similar, but the probes with serine still bound with higher affinity. Notable probes with SS motifs having high binding affinity include SS, SSVFGREP, SSSS, SSVAYQEPA, and SSVAEPA. FIG. 24B shows the distance from the N-terminus for probes having serine/threonine motifs and the corresponding fluorescence signal on the ˜3.2 million probe peptide microarray (V16). FIG. 24C shows the distance from the N-terminus for the di-serine probes and the corresponding fluorescence signal on the ˜125 k probe peptide microarray (V13). FIG. 24D shows the binding affinity for the probes comprising an N-terminal di-serine motif in comparison to other di-residue motifs. FIG. 24E shows the distribution of the number of age-associated features amongst peptide probes having N-terminal motifs that are AS, SA, SS, and WS on the peptide microarray (iCX1).

FIG. 25A-D show that the affinity towards SS was limited to when the N-terminus of the probe was acetylated. FIG. 25A shows the comparison of probes with acetylated N-terminus as opposed free amine N-terminus. FIG. 25B details the fold change in antibody (Ab) binding signal associated with age obtained in young (<40) v (old (>60) donors when binding was performed on arrays of acetylated or non-acetylated peptides. FIG. 25C illustrates that the Ab binding to two types of arrays each comprising two replicate libraries of peptides. In the first array type, both replicate libraries contained features that were acetylated (Ac/Ac); in the second type of array, only library A was acetylated, while library B was non-acetylated (Ac/NH2). The samples used were derived from young (<40) and old (>60) donors. FIG. 25D shows the acetylation/non-acetylation status of the immuno-signature peptides from which the immune index was determined. The data show that the immune index that was determined for two different populations of donors (sf or oc). FIG. 25 shows the accuracy of all probes (FIG. 25E), acetylated probes only (FIG. 25F), and unacetylated probes only in predicting age (FIG. 25G). Both acetylated and unacetylated probes provide similar accuracy (a distinct metric from effect side which acetylation has a significant effect on). Thus, unacetylated probes can still yield an accurate immune index.

FIG. 26A-B illustrate the “immune age” residuals, which were peptide array score that were highly stable across peptide array formats, array synthesis reagents, and model training sets. A single test set is shown in FIG. 26A, and multiple array formats, syntheses, were used to train the Immune Index (x- and y-axes as labeled). Values on x- and y-axes are residuals, which normalize out the default transitive correlation of all models being correlated to chronological age. FIG. 26B depicts the donor samples examined across 6 different combinations of dates of experimentation, batches of wafer, and array types. The residuals remained correlated across all 6 combinations.

FIG. 27 illustrates a regression model yielding similar results across samples binned by body mass index (BMI) (FIG. 27A), sample collection site (FIG. 27B), and ethnicity (FIG. 27C). Each dot was a single donor, and each line showed regression across a set of donors based on distinct binnings. Chronological age was shown on the x-axis, prediction of age based on peptide array was shown on the y-axis. Legend shows correlation coefficient, regression slope, intercept, and number of samples in a given bin (N). Data shown is for a model learned from 1068 and applied to 1116 as a hold-out set; only 1116 data is shown. In each of these studies, the intercept and slope were not statistically significantly different. Similarly, the bin-slope interaction term was not significant.

FIG. 28A-FIG. 28B show how the peptide array age-associated signal requires antibody binding and does not require small molecules. 30 kDa column filters were used to filter the samples. For 30 kDa filters, the flow-through contained only small molecules (<90 kDa). FIG. 28A demonstrates antibody purification methods where IgG was required for machine learning prediction of chronological age. 16 donor samples were selected to obtain coverage of chronological age regression dynamic range. These 16 samples were processed in 4 ways: (1) no processing (sample source), (2) filtered through 30 kDa column and only the filtrate (>˜15 kDa molecules retained; filtrate), (3) filtered through 30 kDa column and only the flow-through retained (<˜75 kDa molecules retained; Flow through), and (4) the filtrate and flow through were recombined after running through column. FIG. 28A shows that the sample source, filtrate +, flow-through, and filtrate all recapitulated original signal. In contrast, the flow-through alone, which was severely IgG depleted, had no correlation with original signal. FIG. 28B shows raw signal being recapitulated, the machine learning regression model was recapitulated only when IgG is present. The 16 samples were plotted as machine learning regression values from the original (x-axis) and filter column-processed (y-axis) data.

FIG. 29 illustrates how the immune index predictive of immune age is constant over 15 months. Each line represents the immune index (FIGS. 29A and 29B) and the SS score derived from the binding of serum Abs from each of 12 different donors at multiple intervals during 450 days. The lines were within a range of 5 years of age over 15 months. FIG. 29C shows the SS score derived from binding to acetylated SS probes.

### DETAILED DESCRIPTION OF THE INVENTION

Disclosed herein are systems and methods for generating predictions of immune function and/or wellness. Biomarkers can be profiled using various assays such as peptide arrays that allow identification of protein binding. Assay signals can be aggregated and analyzed using statistical methods. In some instances, the assay signals are inputted into a trained machine learning algorithm such as a classifier or regression model to generate one or more predictions. The predictions can include scores or indexes of immune health (e.g. IgG diversity, inflammation score, age, BMI, etc). In some cases, the results of the analysis are presented to a user in the form of a report. For example, various forms of information relevant to immune health (e.g. cytokines, immune cell counts/fractions, antibody repertoire) can be summarized into a single metric such as an immune wellness index. In some instances, the machine learning algorithm predicts age, BMI, or a combination thereof using one or more biomarkers. In some instances, age and/or BMI can be used to generate an immune index that incorporates relevant biomarker information to provide a measure of immune wellness. The results of the machine learning algorithm can be provided to a user in a report such as on a phone, website, or printable document (e.g. PDF).

### Machine Learning

Disclosed herein are computer-implemented systems and methods for generating a prediction. In some cases, a prediction is generated by a predictive algorithm. The prediction can be generated using array data such as, for example, peptide array binding data. In some cases, the prediction is for immune health. The algorithm can be a machine learning algorithm such as a classifier or regression model configured to generate a prediction based on a panel of features such as peptide array binding. The classifier can be generated using peptide array data. For example, in some cases, peptide array binding data from a plurality of subjects is processed to generate or train a classifier or regression model comprising a panel of features corresponding to the peptides in the array and that are predictive of immune health, by associating the peptide array data with the actual immune health. Various forms of biological data can be incorporated into these models. For example, peptide array binding data, metabolite data, cytokine data, or any combination thereof can be used. In many cases, a computer-implemented algorithm is used to analyze the peptide array data of the plurality of subjects to generate a classifier or regression model comprising a panel of peptide array features predictive of immune health. The algorithm is often trained using peptide array binding data from a plurality of subjects to determine the association between peptide feature binding and immune health.

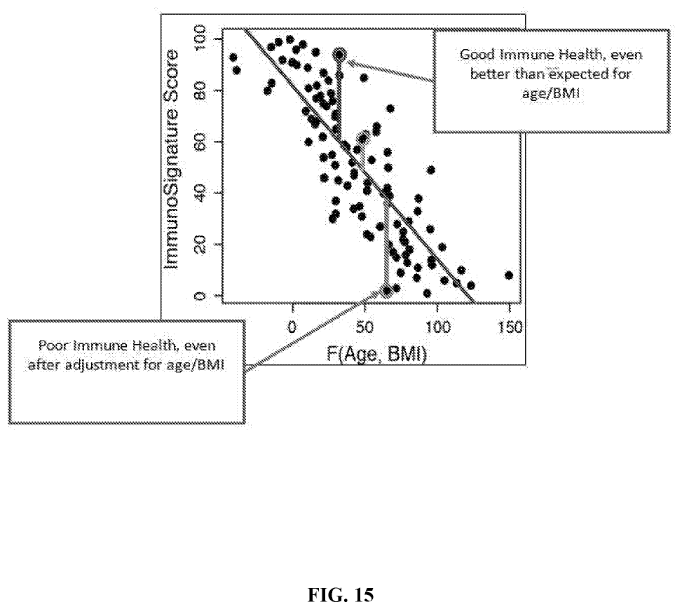

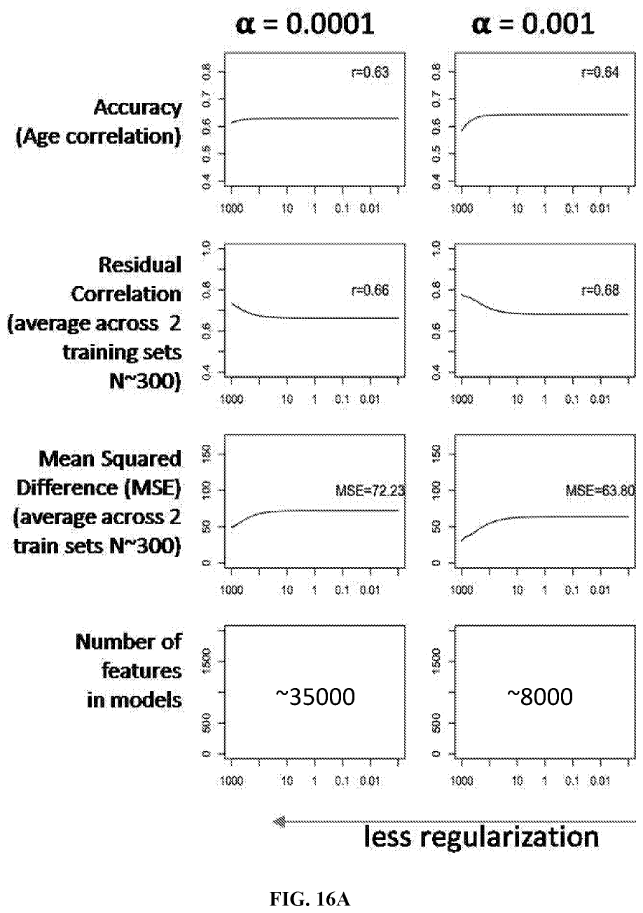

In some instances, the machine learning algorithm predicts age, BMI, or a combination thereof (and/or residuals thereof) using one or more biomarkers. A function of age and BMI (and residuals thereof) may be used to generate the prediction(s). An exemplar function of age and BMI has the formula: Tested F(Age, BMI)=(Age−25)/3+(BMI−18)/1.5. This function gives equal weight and similar dynamic range with the uniformly sampled training set. The immune index or prediction may be plotted against the F(Age, BMI) value obtained to provide a comparison of expected immune health (e.g. based on actual age and BMI) versus predicted immune health (e.g. based on one or more biomarkers). As shown in FIG. 15, the comparison allows a determination of the subject's immune health adjusted for age and BMI.

The algorithm is optionally trained on peptide array data from at least about 50, 100, 150, 200, 300, 400, 500, 600, 700, 800, 900, 1000, 2000, 3000, 4000 or 5000 subjects, including increments therein. In some cases, the algorithm is trained on peptide array data from no more than about 50, 100, 150, 200, 300, 400, 500, 600, 700, 800, 900, 1000, 2000, 3000, 4000 or 5000 subjects, including increments therein. In some cases, the algorithm, after training, achieves near (within 10%) asymptotic accuracy using samples from no more than about 300-500 subjects. In some instances, the trained algorithm achieves within 10% asymptotic accuracy using samples taken from no more than about 100, 200, 300, 400, 500, 600, 700, 800, 900, or about 1000 subjects.

A classifier or trained algorithm as described herein may be used to make a prediction of immune health. One or more selected feature spaces such as peptide array data can be provided to the classifier. Illustrative algorithms can include methods that reduce the number of variables and are selected from a non-limiting group of algorithms including principal component analysis (PCA), partial least squares (PLS) regression, and independent component analysis (ICA). Algorithms can include methods that analyze numerous variables directly and are selected from a non-limiting group of algorithms including methods based on machine learning processes. Machine learning processes can include random forest algorithms, bagging techniques, boosting methods, or any combination thereof. Methods may be statistical methods. Statistical methods can include penalized logistic regression, prediction analysis of microarrays, methods based on shrunken centroids, support vector machine analysis, or regularized linear discriminant analysis.

A classifier described herein comprises at least one feature space. The classifier sometimes comprises two or more feature spaces. The two or more feature spaces may be distinct from one another. Each feature space comprises at least one type of information about a sample, such as, for example, peptide array binding signal, measurement of cytokine levels, and/or measurement of metabolites. The accuracy of the prediction is often improved by combining two or more feature spaces in a classifier or regression model rather than using a single feature space. Individual feature spaces sometimes have different dynamic ranges. The difference in the dynamic ranges between feature spaces can be at least 1, 2, 3, 4, or 5 orders of magnitude. As a non-limiting example, the gene expression feature space sometimes has a dynamic range between 0 and about 200, and the sequence variation feature space may have a dynamic range between 0 and about 20.

In some cases, a feature space comprises a panel of peptide array signals. The panel of peptide array signals of an individual feature space can be associated with a prediction of immune health. In some instances, at least a subset of the features used in the classifiers or regression algorithms for generating the health indicators or metrics disclosed herein comprise the signal for peptides that have one or more serine and/or threonine motifs. In some instances, at least 10%, 20%, 30%, 40%, 50%, 60%, 70%, 80%, 90%, 95%, or 99% or more of the peptide features used in the classifiers or algorithms disclosed herein have at least one serine and/or threonine motif. In some instances, the classifiers or algorithms disclosed herein comprise a peptide feature space comprising a plurality of peptides comprising one or more serine and/or threonine motifs.

In some embodiments, a machine learning algorithm is provided with unlabeled or unclassified data for unsupervised learning, which leaves the algorithm to identify hidden structure amongst the cases (e.g., clustering). In some embodiments, unsupervised learning is used to identify the representations that are most useful for classifying raw data (e.g., identifying features that help separate communications into separate cohorts that may be analyzed using different models and/or evaluated with different thresholds or rules). For example, unsupervised learning is capable of identifying hidden patterns such as relationships between certain features from the data in the knowledge base that would not be readily apparent to a human.

In some embodiments, one or more sets of training data are generated and provided to an emergency location validation system comprising one or more algorithms for making predictions. In some embodiments, an algorithm utilizes a predictive model such as a neural network, a decision tree, a support vector machine, or other applicable model. Using the training data, an algorithm is able to form a classifier for generating a classification or prediction according to relevant features. The features selected for classification can be classified using a variety of viable methods. In some embodiments, the trained algorithm comprises a machine learning algorithm. In some embodiments, the machine learning algorithm is selected from at least one of a supervised, semi-supervised and unsupervised learning, such as, for example, a support vector machine (SVM), a Naïve Bayes classification, a random forest, an artificial neural network, a decision tree, a K-means, learning vector quantization (LVQ), regression algorithm (e.g., linear, logistic, multivariate), regularized regression algorithm (e.g., linear, logistic, multivariate), association rule learning, deep learning, dimensionality reduction and ensemble selection algorithms. In some embodiments, the machine learning algorithm is a support vector machine (SVM), a Naïve Bayes classification, a random forest, or an artificial neural network. Machine learning techniques can include bagging procedures, boosting procedures, random forest algorithms, other meta-learning methods, other ensemble methods, and combinations thereof.

In some embodiments, a machine learning algorithm such as a classifier is tested using data that was not used for training to evaluate its predictive ability. In some embodiments, the predictive ability of the classifier is evaluated using one or more metrics. These metrics include accuracy, specificity, sensitivity, positive predictive value, negative predictive value, which are determined for a classifier by testing it against a set of independent cases. In some instances, an algorithm has an accuracy of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a specificity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a sensitivity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an algorithm has a positive predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances an algorithm has a negative predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some cases, an algorithm has a validation area under curve (AUC) of at least 0.50, 0.55, 0.60, 0.65, 0.70, 0.75, 0.80, 0.85, 0.90, 0.95, or greater than 0.95. In the context of Immune Index, a classifier may discriminate between two sets of samples. These two sets may be old vs young as defined by single cutoff of about 35, 40, 45, 50, 55, 60, or 65 years old or defined by two cutoffs (young are less than 35, 40, 45, 50, 55; old are greater than 40, 45, 50, 55, 60, 65), or similarly defined. The two sets may be high BMI or low BMI as defined by single cutoff of about 18, 25, 30, 35 or defined by two cutoffs (low BMI less than about 20, 25, 30; high BMI are greater than 25, 30, 35, 40), or similarly defined.

In some cases, an immune index (i2) is calculated. The immune index can be compared against an expected immune index (Ei2) is calculated, and the difference between the two is referred to as the residual. The residual is often associated with meaningful immune phenotypes. A key consideration in evaluating the residual is determining the composition of the residual. The residual is typically considered part of an error term of the regression model. For example, regression error is defined by the sum of squared residuals (e.g. deviations predicted from actual empirical values of data). The bias-variance tradeoff is the problem of minimizing both bias and variance as the two source of error in statistical or machine learning modeling. Bias represents the error from incorrect assumptions in the model or learning algorithm. A high bias can be indicative of an underfit model or algorithm that fails to account for relevant relationships between the features and the output (e.g. immune index). On the other hand, variance represents the error from sensitivity to small variations in the training set. A high variance can be indicative of an overfit model or algorithm that is too sensitive to the noise in the training set. For example, models with low bias and high variance may be more complex and able to model the training set with greater accuracy.

The error (e.g. regression error in a regression model) can be broken down into the components of bias, variance, and noise. In some cases, simulations are performed using training and validation sets to estimate each of these each individual contributions to the error. For example, the stability of each sample's residual can be determined (e.g. constant and/or degree of variance) using random samplings of the training set (e.g. 50% random subsamplings, yielding 300 samples per subsampling, which is set below the estimated number of ˜500 samples required for optimal prediction). A high stability or low variance using random samplings would strongly suggest that variance is not a major source of error. Noise can be accounted for by performing technical replicates to determine the scores and correlation values. If the scores and correlation values are virtually identical, then noise is unlikely to be a major source of error. Thus, low variance and noise would suggest that bias is the primary source of error, which indicates that the residual is a meaningful metric for immune, disease, or other human phenotypes associated with the immune score (e.g. immune index).

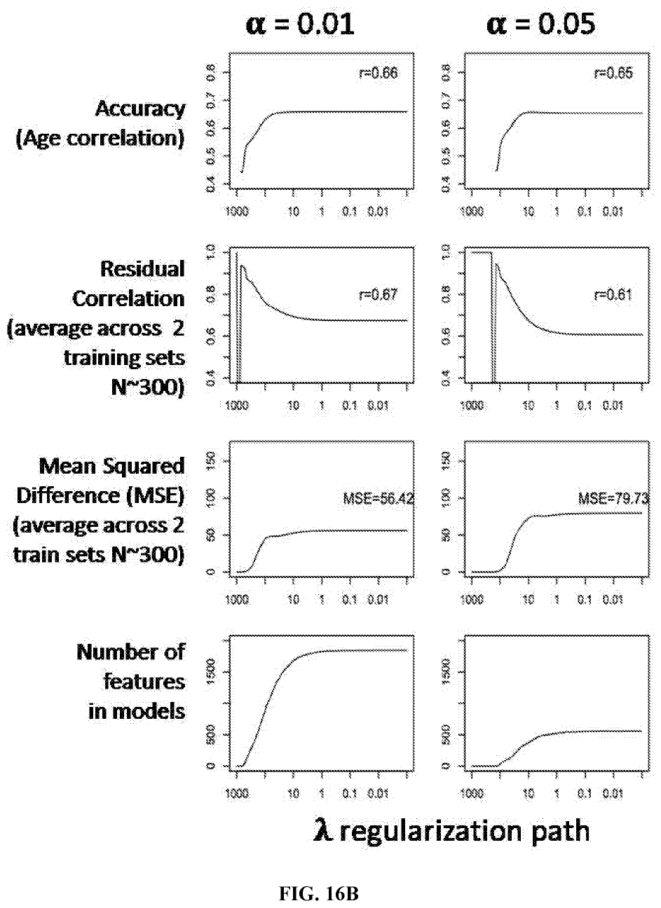

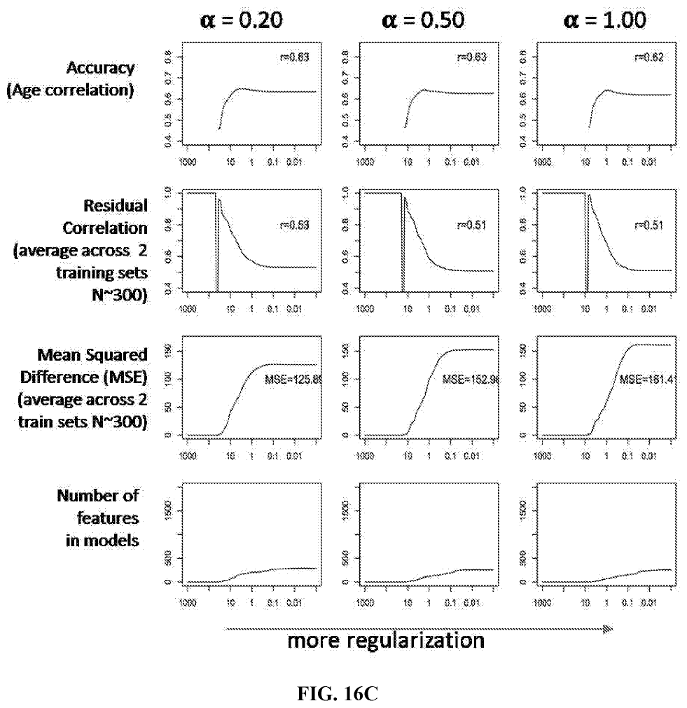

Regularization can be used to alter the residual correlation. In some cases, regularized regression is performed using elastic net. Regularization using elastic net can be carried out at different alpha (a) and lambda (k) values as shown in FIG. 16A, FIG. 16B, and FIG. 16C. In some cases, regularization parameters are assigned values that maximize accuracy and prefer bias over variance. For example, a can be at equal to 0.01 and λ set equal to 1 based on classifier asymptotic stability.

### Immune Health

Disclosed herein are platforms, systems, methods, and devices for generating predictions of immune health. Immune health or wellness can be assayed by profiling biomarkers in a biological sample such as by detecting binding of biomarkers (e.g. immunoglobulins, proteins, biomolecules, etc) to peptide arrays or other available assays. For example, peptide arrays can be characterized by intensity of individual probes, a global diversity metric that summarizes all probes of a given intensity, and/or linear and/or non-linear combinations of probes. Predictions of immune health can include one or more metrics such as IgG diversity, immunity score, immunity-outlier score, inflammation score, immune diversity score, and a summary immune wellness or health metric. These one or more metrics can be generated using models trained according to the machine learning algorithms disclosed herein. For example, data (e.g., peptide array, cytokine, metabolite data) can be labeled with IgG diversity, immunity score, immunity-outlier score, inflammation score, immune diversity score, or a summary immune wellness or health metric and then used to train a classifier or regression model. The trained model (e.g., predictive algorithm) can be used to receive new data for an individual and generate a predictive metric for that individual. Predictions can also include proxy immune wellness metrics such as chronological age, estimates of weight/height/BMI, or other metrics that are associated with immune wellness.

The prediction of immune health based on biomarker information obtained from a subject can be compared to or normalized against an expected immune health (e.g. a score or index determined using actual age and BMI of the subject). The subject's immune health can be benchmarked against the expected immune health to determine or estimate the subject's predicted health based on the subject's age, BMI, or both (or one or more other relevant factors such as cytokine levels). For example, a subject having a poor immune health in an absolute sense compared to the overall population may still have superior immune health relative to others in the subject's age group.

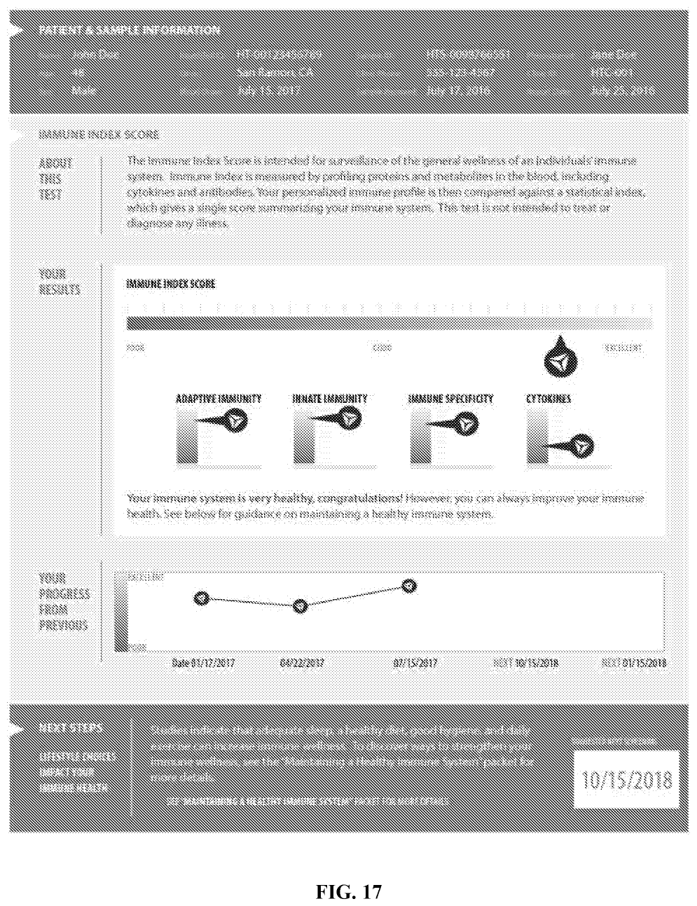

The immune health or wellness of a subject can be presented as a report. For example, one or more immune wellness scores can be presented in a report to consumers that can be printed out onto paper, viewed as PDF through website or on desktop, and/or through a phone app. An exemplar report is shown in FIG. 17. Subject and/or sample information can be included in the report such as name, age, sex, ID, clinic, blood draw date, sample ID, clinic phone, sample receipt date, phlebotomist, clinic ID, and report date. The report can comprise an immune index score that is intended for surveillance of the general wellness of an individuals' immune system. The Immune Index is measured by profiling proteins and/or metabolites in the blood, e.g. antibodies and/or cytokines. The personalized immune profile is compared against a statistical index, which gives a single score summarizing the subject's immune system. For example, FIG. 17 shows an overall immune index score as well as scores for adaptive immunity, innate immunity, immune specificity, and cytokines, respectively. In addition, the report may track and show progress in the immune health metric(s) over time. In some cases, the report also contains instructions or recommendations for the subject such as lifestyle choices/changes. Examples of lifestyle changes include changes to diet, exercise, sleep, and stress management. Sometimes, the report has advice and/or links to additional information or external resources for strengthening immune wellness. The report may also suggest a time for the next screening.

### Immune Health Portal

Disclosed herein are platforms, systems, methods, and devices providing an immune health portal allowing users to access their immune health predictions. The immune health portal has user data that is generally secured through encryption and requires user authentication for user login. In some cases, user authentication is provided by requiring a login password and/or biometric identification (e.g. fingerprint, iris or retinal scan, voice recognition, facial recognition). The immune health portal can comprise one or more software modules. In some cases, the immune health portal comprises a software module for receiving user input parameters. These input parameters can include user information such age, gender, ethnicity, BMI, health conditions, medical history, diet, exercise, geographic location and/or climate, and other parameters relevant to immune health. A user is able to obtain personalized immune health predictions that are tailored according to entered input parameters. The immune health portal may provide one or more predictive algorithms trained according to specific subsets of data corresponding to certain combinations of input parameters. For example, different predictive algorithms may be generated for distinct populations (e.g. based on ethnicity or diet). This approach controls for differences in immune health that arise from specific population characteristics that are not generalizable to the overall population. As another example, people who have contracted chicken pox in their childhood are at risk of shingles later in life, which may be accompanied with various complications. Accordingly, a user who enters a positive parameter for chicken pox may receive an immune health indicator that generated by a machine learning algorithm or classifier trained using data from individuals who had contracted chicken pox.

The immune health portal can comprise a software module obtaining a machine learning algorithm and a software module applying the machine learning algorithm to analyze immunological measurements of the user to generate a prediction of immune health. In some cases, the software module obtains a machine learning algorithm adapted to the user input parameters. The machine learning algorithm may be selected from a plurality of machine learning algorithms, wherein each of the plurality of machine learning algorithms is customized to a combination of user input parameters.

The immune health portal may be accessed by a variety of methods including, but not limited to, a web portal, a mobile application, or a software application. In some cases, the immune health portal generates an immune health report that is delivered to a user by electronic communication such as, for example, e-mail, web messaging, SMS, MMS, or electronic chat. Such electronic communications are generally secured through encryption.

The immune health portal can interface with one or more databases storing user data such as immune health predictions (current and historical). The one or more databases may be stored on a server and/or cloud-based network. In some cases, the immune health portal provides a search tool for keyword and/or parameter-based searching of the one or more databases.

### Immunological Measurements

Methods of determining immune health provided herein, in certain instances rely upon measurements of immunological or biological markers or parameters obtained from a subject or a population of subjects. Such measurements of immunological or biological markers or parameters include, but are not limited to, for example, measurement or detection of antibody peptide binding, immune protein sequences, cytokine levels, metabolite levels, blood cell counts, including immunological cell counts, RNA sequences, mRNA sequences, microRNA sequences, microbiome composition or diversity statistics, and combinations thereof.

### Peptide Binding

Methods of measuring an immune health in an individual or subject provided herein comprise obtaining an immunological or biological measurement from a biological sample from the subject, in some embodiments, comprising, for example, antibody-peptide binding. Antibody peptide binding comprises, in some cases, determining the binding of antibodies from a biological sample from the subject to one or more peptides, for example, by measuring binding of antibodies from the subject to a population of peptides. In some cases, antibody binding to the population of peptides is measured, for example by using a population of peptides immobilized onto a substrate. In some instances, the population of peptides are immobilized onto beads. In some instances, the population of peptides are immobilized in wells on, for example, a microtiter plate. In yet other instances, the population of peptides are spotted or synthesized onto, for example, an array, such as a microarray. In some cases, the array comprises a plurality of peptides that correlate with known proteins in a proteomic space. In yet other instances, the array comprises a plurality of peptides that are uncorrelated with known proteins in a proteomic space. In still other instances, the array comprises a plurality of a mixture of peptides that correlate with known proteins in a proteomic space and uncorrelated with known proteins in a proteomic space.

In some instances, peptide microarrays useful in methods of measuring immune heath in a subject provided herein comprise a population of peptides that are spotted on a microarray, synthesized on a microarray, or a combination thereof. Binding of antibodies from the subject to the peptides may be visualized using a variety of methods including but not limited to fluorescence, chemiluminescence, colorimetric, and other visualization methods detectable by visual and other means.

In some instances, measurement of antibody-peptide binding includes use of an immunosignature assay, the assay comprising a. contacting a peptide array with a first biological sample from an individual or subject; b. detecting binding of antibodies in the first biological sample with the peptide array to obtain a first immunosignature profile; c. contacting a peptide array with a control sample derived from one or more individuals with, for example, a known antibody panel or other marker; d. detecting binding of antibody in the control sample with the peptide array to obtain a second immunosignature profile; e. comparing the first immunosignature profile to the second immunosignature profile to determine if an individual or subject has detectable and/or measurable levels of a specific or multiple antibody-peptide binding events. In some embodiments, the method performance of the immunosignature assay is characterized by an area under the receiver operator characteristic (ROC) curve (AUC) being greater than 0.6, greater than 0.7, greater than 0.8, greater than 0.9 or greater than 0.95.

In some instances, peptides are identified from the immunosignature binding patterns obtained, which present patterns or motifs indicative of an individual or subject's immune health. In some instances, individuals and subjects are screened for the antibody binding to at least one, at least two, at least three, at least four, at least five, at least six, at least seven, at least eight, at least nine, or at least ten different peptide sequence motifs. In addition, enrichment of at least one specific amino acid can also be isolated, which are indicative of an individual or subject's immune health. In some instance, sequence motifs can be enriched in at least one amino acid by at least 100%, at least 125%, at least 150%, at least 175%, at least 200%, at least 225%, at least 250%, at least 275%, at least 300%, at least 350%, at least 400%, at least 450%, or at least 500%.

In some embodiments, the peptides on the microarray comprise natural amino acids. In some embodiments, the peptides on the microarray comprise non-natural amino acids, or a mixture of natural and non-natural amino acids. In yet other instances, the non-natural amino acids include but are not limited to D-amino acids, homo amino acids, beta-homo amino acids, N-methyl amino acids, alpha-methyl amino acids, deiminated amino acids, non-natural side chain variant amino acids and other non-natural amino acids.

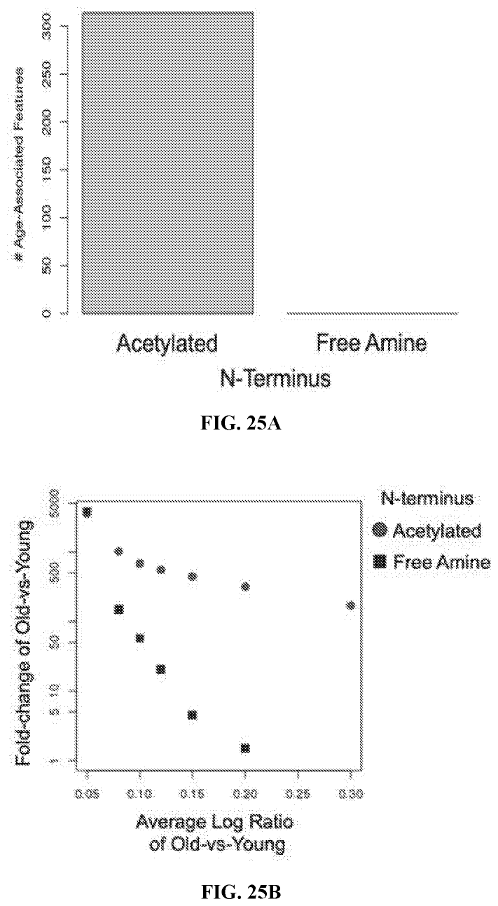

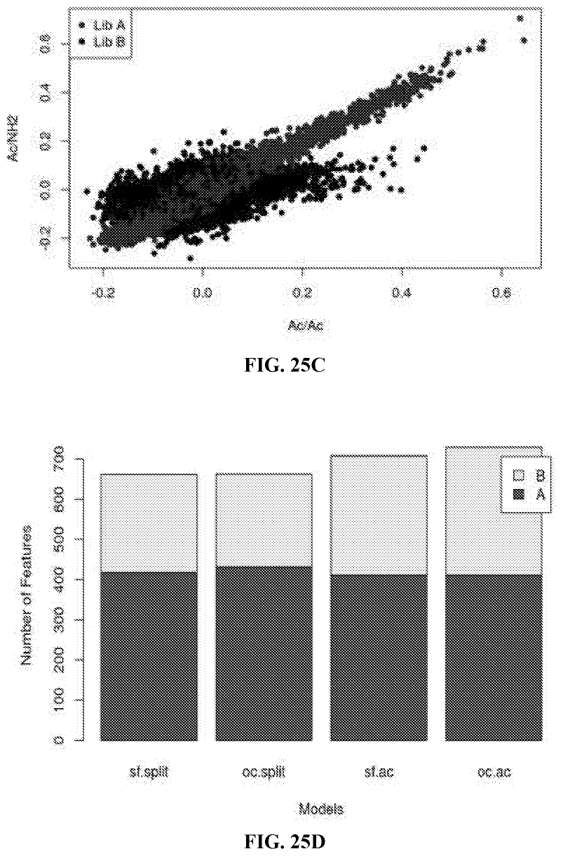

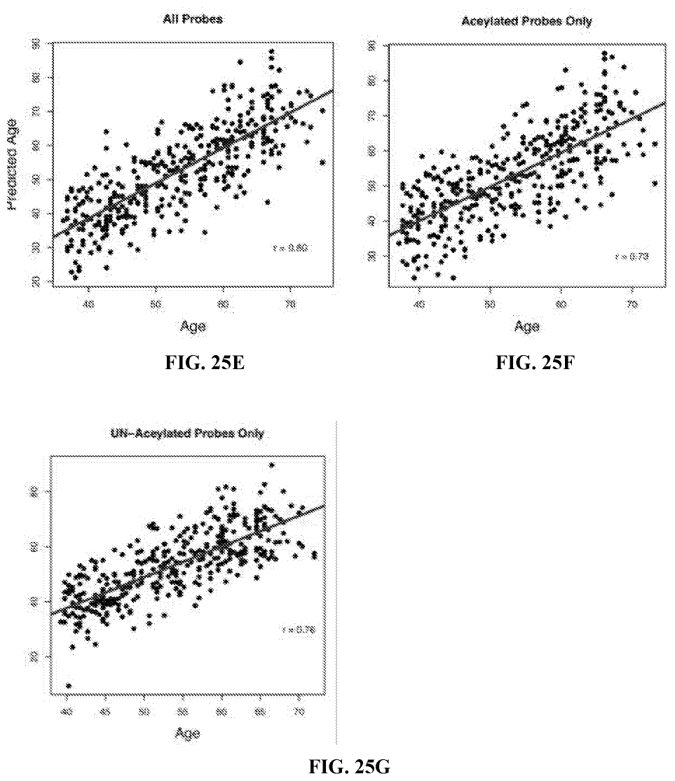

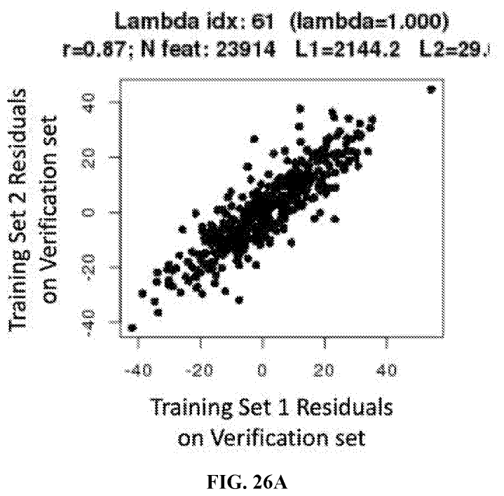

The present disclosure has discovered that acetylation of the peptide probes on the microarray significantly improves signal to noise. FIGS. 25A-25D show that acetylated probes provide a substantially higher effect size compared to unacetylated probes. The acetylation can be N-terminal acetylation. In some instances, the peptides on the microarray are acetylated. In some instances, the peptides are not acetylated. In some instances, at least about 1%, 2%, 3%, 4%, 5%, 10%, 20%, 30%, 40%, 50%, 60%, 70%, 80%, 90%, or 95% or more of the peptides on the microarray are acetylated. In some instances, the predictive model that generates a metric of immune health (e.g., immune wellness score) utilizes a panel of peptides from the microarray that are acetylated. In some cases, at least about 1%, 2%, 3%, 4%, 5%, 10%, 20%, 30%, 40%, 50%, 60%, 70%, 80%, 90%, 95%, or 100% of the panel of peptides used in the predictive model are acetylated. In some cases, the panel of peptides comprises an acetylation percentage of 10% to 100%. In some cases, the panel of peptides comprises an acetylation percentage of 10% to 20%, 10% to 30%, 10% to 40%, 10% to 50%, 10% to 60%, 10% to 70%, 10% to 80%, 10% to 90%, 10% to 95%, 10% to 100%, 20% to 30%, 20% to 40%, 20% to 50%, 20% to 60%, 20% to 70%, 20% to 80%, 20% to 90%, 20% to 95%, 20% to 100%, 30% to 40%, 30% to 50%, 30% to 60%, 30% to 70%, 30% to 80%, 30% to 90%, 30% to 95%, 30% to 100%, 40% to 50%, 40% to 60%, 40% to 70%, 40% to 80%, 40% to 90%, 40% to 95%, 40% to 100%, 50% to 60%, 50% to 70%, 50% to 80%, 50% to 90%, 50% to 95%, 50% to 100%, 60% to 70%, 60% to 80%, 60% to 90%, 60% to 95%, 60% to 100%, 70% to 80%, 70% to 90%, 70% to 95%, 70% to 100%, 80% to 90%, 80% to 95%, 80% to 100%, 90% to 95%, 90% to 100%, or 95% to 100%. In some cases, the panel of peptides comprises an acetylation percentage of 10%, 20%, 30%, 40%, 50%, 60%, 70%, 80%, 90%, 95%, or 100%. In some cases, the panel of peptides comprises an acetylation percentage of at least 10%, 20%, 30%, 40%, 50%, 60%, 70%, 80%, 90%, or 95%. In some cases, the panel of peptides comprises an acetylation percentage of at most 20%, 30%, 40%, 50%, 60%, 70%, 80%, 90%, 95%, or 100%. In some cases, at least about 1%, 2%, 3%, 4%, 5%, 10%, 20%, 30%, 40%, 50%, 60%, 70%, 80%, 90%, 95%, or 100% of the features used in the predictive model are acetylated peptides. For example, a predictive model can include both peptide signals from a microarray and cytokine signals from a different assay (e.g., mass spectrometry analysis of a serum sample), and a percentage or proportion of the signals utilized in the model can be from acetylated peptides.

The present disclosure recognizes a surprising and unexpected discovery that peptide probes characterized by certain amino acid motifs on the microarray tend to provide significantly higher signal relative to other peptides. Specifically, serine, threonine, and serine/threonine motifs have been found to provide surprisingly high fluorescence intensity on various peptide microarrays disclosed herein. Accordingly, in some instances, some of the peptides on the microarray comprise peptides that have one or more serine (S) and/or threonine (T) motifs. In some instances, some of the peptides on the microarray comprise peptides that have one or more serine (S) motifs. In some instances, the peptides comprise an S, SS, SSS, or SSSS motif. In some instances, the peptides comprise an S, SS, SSS, SSSS, ST, or TS motif. In some instances, the peptides on the microarray comprise peptides that have one or more threonine (T) motifs. In some instances, the peptides comprise a T, TT, or TTT motif. In some instances, the peptides on the microarray comprise peptides that have a serine-threonine motif. In some instances, the peptides comprise a TS or ST motif. In some instances, the peptides on the microarray comprise peptides that have one or more serine and/or threonine motifs that are located on the N-terminus. In some instances, the one or more serine and/or threonine motifs are located no more than 1 amino acid away the N-terminus (N-1). In some instances, the one or more serine and/or threonine motifs are located 2 amino acids no more than 2 amino acids away from the N-terminus (N-2). In some instances, the one or more serine and/or threonine motifs are located no more than 1 amino acid, 2 amino acids, 3 amino acids, 4 amino acids, 5 amino acids, or 6 amino acids away from the N-terminus. In some instances, one or more peptides having one or more of these motifs make up at least a subset of the features used in the peptide panel for the predictive algorithms disclosed herein (e.g., classifiers and regression models for generating immune health or wellness metrics).

In some instances, at least a subset of the features used in the classifiers or regression algorithms for generating the health indicators or metrics disclosed herein comprise the signal for peptides that have one or more serine and/or threonine motifs. In some instances, at least 10%, 20%, 30%, 40%, 50%, 60%, 70%, 80%, 90%, 95%, or 99% or more of the peptide features used in the classifiers or algorithms disclosed herein have at least one serine and/or threonine motif. In some instances, the classifiers or algorithms disclosed herein comprise a peptide feature space of peptides comprising a plurality of serine and/or threonine motifs. In some instances, the plurality of serine and/or threonine motifs comprise multi-serine motifs (e.g., S, SS, SSS, SSSS). In some instances, the plurality of serine and/or threonine motifs comprise multi-serine motifs (e.g., SS, SSS, SSSS). In some instances, the plurality of serine and/or threonine motifs comprise multi-threonine motifs (e.g., TT, TTT). In some instances, the plurality of serine and/or threonine motifs comprise serine and serine/threonine motifs (e.g., S, SS, SSS, SSSS, TS, ST). In some instances, the plurality of serine and/or threonine motifs comprise serine, threonine, and serine/threonine motifs (e.g., S, SS, SSS, SSSS, TS, ST, TTT, TT, T). In some instances, the peptide panel comprises peptides that have one or more serine and/or threonine motifs that are located on or near the N-terminus. In some instances, the one or more serine and/or threonine motifs are located no more than 1 amino acid away the N-terminus (N-1). In some instances, the one or more serine and/or threonine motifs are located 2 amino acids no more than 2 amino acids away from the N-terminus (N-2). In some instances, the one or more serine and/or threonine motifs are located no more than 1 amino acid, 2 amino acids, 3 amino acids, 4 amino acids, 5 amino acids, or 6 amino acids away from the N-terminus.

In some instances, the machine learning algorithm comprises a feature set comprising a plurality of peptides. In some instances, the plurality of peptides comprises one or more peptides having at least one serine and/or threonine motif. In some instances, peptides having a serine and/or threonine motif make up a percentage of the peptide panel that is about 10% to about 100%. In some instances, peptides having a serine and/or threonine motif make up a percentage of the peptide panel or peptide feature space that is about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 10% to about 100%, about 20% to about 30%, about 20% to about 40%, about 20% to about 50%, about 20% to about 60%, about 20% to about 70%, about 20% to about 80%, about 20% to about 90%, about 20% to about 95%, about 20% to about 100%, about 30% to about 40%, about 30% to about 50%, about 30% to about 60%, about 30% to about 70%, about 30% to about 80%, about 30% to about 90%, about 30% to about 95%, about 30% to about 100%, about 40% to about 50%, about 40% to about 60%, about 40% to about 70%, about 40% to about 80%, about 40% to about 90%, about 40% to about 95%, about 40% to about 100%, about 50% to about 60%, about 50% to about 70%, about 50% to about 80%, about 50% to about 90%, about 50% to about 95%, about 50% to about 100%, about 60% to about 70%, about 60% to about 80%, about 60% to about 90%, about 60% to about 95%, about 60% to about 100%, about 70% to about 80%, about 70% to about 90%, about 70% to about 95%, about 70% to about 100%, about 80% to about 90%, about 80% to about 95%, about 80% to about 100%, about 90% to about 95%, about 90% to about 1000, or about 95% to about 100%. In some instances, peptides having a serine and/or threonine motif make up a percentage of the peptide panel that is about 10%, about 20%, about 30%, about 40%, about 50%, about 60%, about 70%, about 80%, about 90%, about 95%, or about 100%. In some instances, peptides having a serine and/or threonine motif make up a percentage of the peptide panel that is at least about 10%, about 20%, about 30%, about 40%, about 50%, about 60%, about 70%, about 80%, about 90%, or about 95%. In some instances, peptides having a serine and/or threonine motif make up a percentage of the peptide panel that is at most about 20%, about 30%, about 40%, about 50%, about 60%, about 70%, about 80%, about 90%, about 95%, or about 100%.

In some instances, the machine learning algorithm has a feature space comprising a plurality of peptides (e.g., from a peptide microarray). In some instances, the plurality of peptides includes peptides characterized by at least one serine and/or threonine motif. In some embodiments, the peptide feature space or peptide panel comprises at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 or more peptides characterized by at least serine and/or threonine motif. In some instances, the peptide feature space or peptide panel comprises no more than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 or more peptides characterized by at least serine and/or threonine motif. In some instances, the peptide feature space comprises at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000 or more peptides, wherein at least 10%, 20%, 30%, 40%, 50%, 60%, 70%, 80%, 90%, 95%, or 100% of the peptides in the feature space are characterized by at least one serine and/or threonine motif. The at least one serine and/or threonine motif can be limited to serine motifs, threonine motifs, or serine/threonine motifs, or any combination thereof. In some instances, the serine and/or threonine motifs are no more than 1, 2, 3, 4, 5, or 6 amino acids from the N-terminus of their respective peptides (for the motif that is closest to the N-terminus).

In some instances, the peptides having serine and/or threonine motifs are acetylated. The acetylation can enhance the signal or effect size of these peptide probes that are already characterized by increased signal, thereby generating a combined effect.

In some instances, the peptides on the microarrays comprise peptides that are at least 4 mer, at least 5 mer, at least 6 mer, at least 7 mer, at least 8 mer, at least 9 mer, at least 10 mer, at least 11 mer, at least 12 mer, at least 13 mer, at least 14 mer, at least 15 mer, at least 16 mer, at least 17 mer, at least 18 mer, at least 19 mer, at least 20 mer, at least 21 mer, at least 22 mer, at least 23 mer, at least 24 mer, at least 25 mer or longer. In some instances, the peptides on the microarrays comprise individual peptides that are equivalent in length. In other instances, the peptides on the microarrays comprise individual peptides that are not equivalent in length. In still other instances, the peptides on the microarrays comprise peptides that are between 5-50 mer, between 5-40 mer, between 5-35 mer, between 5-30 mer, between 5-25 mer, between 5-20 mer or between 5-15 mer.

Peptide microarrays herein comprise peptides spotted or synthesized on the array using a variety of suitable methods. Peptide microarrays comprise a range of spotted or synthesized peptides from about 100 peptides to about 10,000,000 peptides. In some cases, peptide microarrays comprise from about 1,000 peptides to about 100,000 peptides. In some cases, peptide microarrays comprise from about 5,000 peptides to about 50,000 peptides. In some cases, peptide microarrays comprise at least about 1,000 peptides, at least about 2,000 peptides, at least about 3,000 peptides, at least about 4,000 peptides, at least about 5,000 peptides, at least about 6,000 peptides, at least about 7,000 peptides, at least about 8,000 peptides, at least about 9,000 peptides, at least about 10,000 peptides, at least about 11,000 peptides, at least about 12,000 peptides, at least about 13,000 peptides, at least about 14,000 peptides, at least about 15,000 peptides, at least about 16,000 peptides, at least about 17,000 peptides, at least about 18,000 peptides, at least about 19,000 peptides, at least about 20,000 peptides, at least about 21,000 peptides, at least about 22,000 peptides, at least about 23,000 peptides, at least about 24,000 peptides, at least about 25,000 peptides, at least about 26,000 peptides, at least about 27,000 peptides, at least about 28,000 peptides, at least about 29,000 peptides, at least about 30,000 peptides, at least about 31,000 peptides, at least about 32,000 peptides, at least about 33,000 peptides, at least about 34,000 peptides, at least about 35,000 peptides, at least about 36,000 peptides, at least about 37,000 peptides, at least about 38,000 peptides, at least about 39,000 peptides, at least about 40,000 peptides, at least about 41,000 peptides, at least about 42,000 peptides, at least about 43,000 peptides, at least about 44,000 peptides, at least about 45,000 peptides, at least about 46,000 peptides, at least about 47,000 peptides, at least about 48,000 peptides, at least about 49,000 peptides, at least about 50,000 peptides, at least about 51,000 peptides, at least about 52,000 peptides, at least about 53,000 peptides, at least about 54,000 peptides, at least about 55,000 peptides, at least about 56,000 peptides, at least about 57,000 peptides, at least about 58,000 peptides, at least about 59,000 peptides, at least about 60,000 peptides, at least about 61,000 peptides, at least about 62,000 peptides, at least about 63,000 peptides, at least about 64,000 peptides, at least about 65,000 peptides, at least about 66,000 peptides, at least about 67,000 peptides, at least about 68,000 peptides, at least about 69,000 peptides, at least about 70,000 peptides, at least about 71,000 peptides, at least about 72,000 peptides, at least about 73,000 peptides, at least about 74,000 peptides, at least about 75,000 peptides, at least about 76,000 peptides, at least about 77,000 peptides, at least about 78,000 peptides, at least about 79,000 peptides, at least about 80,000 peptides, at least about 81,000 peptides, at least about 82,000 peptides, at least about 83,000 peptides, at least about 84,000 peptides, at least about 85,000 peptides, at least about 86,000 peptides, at least about 87,000 peptides, at least about 88,000 peptides, at least about 89,000 peptides, at least about 90,000 peptides, at least about 91,000 peptides, at least about 92,000 peptides, at least about 93,000 peptides, at least about 94,000 peptides, at least about 95,000 peptides, at least about 96,000 peptides, at least about 97,000 peptides, at least about 98,000 peptides, at least about 99,000 peptides, at least about 100,000 peptides, at least about 1,000,000 peptides, at least about 3,000,000, or at least about 10,000,000 peptides.

Antibody-peptide binding herein comprises measuring binding of antibodies from a subject, for example antibodies found in a biological sample from a subject. Biological samples for antibody-peptide binding measurement herein include any suitable biological sample comprising antibodies that is obtainable from a subject. Suitable biological samples include but are not limited to blood, serum, whole blood, dried blood, plasma, saliva, urine, lymph fluid, semen, vaginal secretions, synovial fluid, tears, bone marrow, cerebrospinal fluid, bronchial secretions, and combinations thereof. In some embodiments, suitable biological samples include serum, whole blood, dried blood, plasma, saliva, and combinations thereof. Biological samples from the subject comprise antibodies including but not limited to IgG, IgA, IgD, IgM, and combinations thereof.

### Cytokines

Methods of measuring an immune health in a subject provided herein comprise obtaining an immunological measurement from a biological sample from the subject, in some embodiments, comprising cytokine levels. Cytokines are small proteins involved in cell signaling pathways including but not limited to autocrine signaling, paracrine signaling, endocrine signaling, and immune signaling. Cytokines include but are not limited to chemokines, interferons, interleukins, lymphokines, and tumor necrosis factors. Multiple types of cells produce cytokines including but not limited to macrophages, B cells, T cells, mast cells, endothelial cells, fibroblasts, and stromal cells. Cytokines useful for measuring immune health include but are not limited to TNFα, GM-CSF, MCP-1 (CCL2), MCP-3, IFNα, IFNγ, IL1β, IL2, IL4, IL5, IL6, IL7, IL8, IL10, IL12, IL13, IL17, IL18, IL21, CRP, EGFR, IP10 (CXCL10), Eotaxin (CCL11), MIG, AGP, sTNF-RI, sTNF-RII, sL2RA, sIL1RA, sIL1RII, sIL6R, CD40L, IL18BP, EGF, VEGF, resistin, leptin, adiponectin, alpha-1-antitrypsin, free fatty acids, and combinations thereof. In some cases, cytokines include CD40L, EGF, Eotaxin (CCL11), GM-CSF, IFNα, IFNγ, IL-1β, sIL-RA, sIL-2R, IL-6, IP-10 (CXCL10), MCP-1 (CCL2), TNFα, sTNF-RI, sTNF-RII, and combinations thereof. In some cases cytokines include Eotaxin (CCL11), sIL-1RA, sIL-2R, sTNF-RI, IP10 (CXCL10), TNFα, IFNα, IFNγ, IL6, sTNF-RII, IL-1β, and combinations thereof.

Methods herein determine cytokine levels in a biological sample from a subject.

Biological samples for cytokine measurement herein include any suitable biological sample comprising cytokines that is obtainable from a subject. Suitable biological samples include but are not limited to blood, serum, whole blood, dried blood, plasma, saliva, urine, lymph fluid, semen, vaginal secretions, synovial fluid, tears, bone marrow, cerebrospinal fluid, bronchial secretions, and combinations thereof. In some embodiments, suitable biological samples include serum, whole blood, dried blood, plasma, saliva, and combinations thereof. In some cases, cytokine levels are measured in cells obtained from a subject, including but not limited to lymphocytes, peripheral blood mononuclear cells, T cells, B cells, macrophages, mast cells, and combinations thereof.

Methods herein determine cytokine levels using any suitable assay. Suitable assays for measurement of cytokines in determining immune health include but are not limited to a bead assay, an aptamer assay, an ELISA assay, and an ELISPOT assay. In some cases, cytokine assays include intracellular cytokine staining, immunofluorescence, quantitative PCR, Western blot, and combinations thereof. In some cases, the assay measures a single cytokine per assay. In some cases, the assay measures multiple cytokines in a multiplex assay. In some cases, methods of measuring cytokines comprise a colorimetric, fluorescent, chemiluminescent, enzymatic, or other measurement.

### Cell Counts

Methods of measuring an immune health in a subject provided herein comprise obtaining an immunological measurement from a biological sample from the subject, in some embodiments, comprising cell counts. Suitable biological samples include but are not limited to blood, whole blood, dried blood, plasma, saliva, urine, lymph fluid, semen, vaginal secretions, synovial fluid, tears, bone marrow, cerebrospinal fluid, bronchial secretions, and combinations thereof. Cell counts herein comprise counting the numbers and types of cells in a biological sample from a subject. In some cases, types of cells counted include but are not limited to red blood cells, white blood cells, platelets, lymphocytes, monocytes, macrophages, mast cells, neutrophils, eosinophils, basophils, T cells, B cells, and combinations thereof. In some cases, CD4+ T cells, CD8+ T cells, CD4+CD25+ T cells, and marker defined subsets (e.g., CD45RA, CD45RA, CCR7, CXCR3, PD1) thereof, are counted.

Cell counts for methods of measuring immune health in a subject comprise any suitable cell counting method. Cell counting methods herein include but are not limited to use of coulter counters, fluorescence activated flow cytometry, flow cytometry, hemocytometers, and combinations thereof. In some cases, cells are stained with a dye before counting. In some instances, cells are stained with antibodies, including fluorescent antibodies and other labeled antibodies before counting. In some cases, cells are magnetically sorted before counting.

### Immune Protein Sequence

Methods of measuring an immune health in a subject provided herein comprise obtaining an immunological measurement from a biological sample from the subject, in some embodiments, comprising obtaining an immune protein sequence. Immune proteins having useful sequence information herein include but are not limited to immunoglobulin proteins, T cell receptor proteins, major histocompatibility complex proteins, and combinations thereof. Immune proteins herein often comprise hypervariable regions which, in some cases, influence an immune response, including an immune response to a pathogen or an immune response to a self-antigen. In some cases, the level or extent of diversity of immune protein sequences in a subject, indicates the degree of diversity of targets recognized by an immune system in the subject, which in turn can be an indicator of immune health. Accordingly, measurement of an immune protein sequence, including the level or extent of diversity of immune protein sequences in an individual or subject may be used in the evaluation of immune health.

Methods of determining an immune protein sequence for methods of measuring immune health in a subject herein alternately comprises obtaining a nucleic acid sequence or a protein sequence. Nucleic acid sequences useful in methods herein include DNA and RNA sequencing methods including but not limited to Sanger sequencing, single molecule real-time sequencing, Ion Torrent sequencing, pyrosequencing, sequencing by synthesis, sequencing by ligation, nanopore sequencing, massively parallel signature sequencing, polony sequencing, DNA nanoball sequencing, and other suitable nucleic acid sequencing methods. Alternately, sequencing immune proteins comprises protein sequencing methods including but not limited to Edman degradation, mass spectrometry, MALDI-TOF mass spectrometry, peptide mass fingerprinting, and other suitable protein sequencing methods.

### Metabolites

Methods of measuring an immune health in a subject provided herein comprise obtaining an immunological measurement from a biological sample from the subject, in some embodiments, comprising metabolite levels. Metabolite levels useful in measuring immune health in a subject comprise intermediate products in metabolism. Exemplary metabolites include but are not limited to fatty acids, amino acids, sugars, enzyme substrates, and combinations thereof. Further examples of metabolites include 1-methyl-adenosine, 1-methyl-guanosine, arginine, asparagine, camitine, glutamate, glutarate, histidine, methionine, methyl-histidine, my-inositol, phenylalanine, tryptophan, tyrosine, ADP, ATP, creatine, diphospho-glycerate, fructose-6-phosphate, glucose-6-phosphate, glutamate, glutathione, malate, N-acetyl-D-glucosamine, NAD+, Phosphoglycerate, UDP-glucose, phosphoglycerate, UDP-glucose, urate, acetyl-camitine, oopthalmic acid, phosphocreatine, propionyl-camitine, adenine, aspartate, ergothioneine, GDP-glucose, GMP, N-acetyl-glutamate, nicotinamide, S-methyl-ergothioneine, sedoheptulose-7-phosphate, succinate, trimethyl-histidine, UDP-acetyl-glucosamine, UDP-glucuronate, camosine, dimethyl-proline, glyceraldehyde-3-phosphate, pantohenate, S-adenosyl-homocysteine, s-adenosyl-methionine, tetradecanoyl-camitine, UMP, trimethyl-phenylalanine, trimethyl-tryptophan, trimethyl-tyrosine, 1,5-anhydroglucitol, adenosine, arginine-succinate, CDP-choline, CDP-ethanolamine, dimethyl-guanosine, dimethyl-lysine, glucose, glucosamine, glycerate, glycerol-phosphocholine, N₆-acetyl-lysine, kynurenine, omithine, proline, threonine, UTP, valine, 2-oxoglutarate, betaine, dimethyl-xanthine, indoxyl-sulfate, N₂-acetyl-lysine, N-acetyl-arginine, N-acetyl-aspartate, N-acetyl-iso-leucine, xanthine, caffeine, hexanoyl-camitine, methyl-lysine, 4-amino-benzoate, 4-guanidino-butanoate, acetylcamosine, cheno-deoxycholate, decanoyl-camitine, dodecanoyl-camitine, glycocheno-deoxycholate, hippurate, isovaleryl-camitine, N-acetyl-omithine, octanoyl-camitine, quinolinic acid, and other suitable metabolites for measuring immune health.

Assays for measuring metabolites for methods of measuring immune health provided herein include measuring metabolites in a biological sample obtained from a subject. Biological samples suitable for measuring metabolites include but are not limited to blood, serum, whole blood, dried blood, plasma, saliva, urine, lymph fluid, semen, vaginal secretions, synovial fluid, tears, bone marrow, cerebrospinal fluid, bronchial secretions, and combinations thereof. In some cases, biological samples for measuring metabolites comprise blood samples obtained from the subject. Assays for measuring metabolites include but are not limited to chromatography, liquid chromatography, mass spectrometry, liquid chromatography-mass spectrometry, ELISA, enzymatic assay, luminescence assay, fluorescence assay, colorimetric assay, and combinations thereof.

### Companion Methods of Prediction, Detection, Diagnosis, Prognosis, or Selection of Treatment Based on Assessment of Immune Health

In some embodiments, a subject's immune health can indicate a presence or absence of at least one of autoimmune diseases, inflammatory diseases, immunodeficiency diseases, or cancer. In some cases, the subject's immune health can indicate a likelihood or risk of developing at least one of autoimmune diseases, inflammatory diseases, immunodeficiency diseases, or cancer. In other cases, the subject's immune health indicates a diagnosis or prognosis of at least one of autoimmune diseases, inflammatory diseases, immunodeficiency diseases, or cancer. In some embodiments, the subject's immune health can be indicative of how the subject responds to a treatment at least one of autoimmune diseases, inflammatory diseases, immunodeficiency diseases, or cancer. In some cases, how the subject responds to a treatment can natural, beneficial, or adverse.

In some cases, the subject can be advised to adopt a lifestyle that decreases the likelihood of developing the at least one of autoimmune diseases, inflammatory diseases, immunodeficiency diseases, or cancer. In some embodiments, the subject is advised to undergo additional diagnostic testing based on the subject's immune health. In some instances, the methods disclosed herein comprise providing a recommendation based on the immune health of the subject. In some instances, the recommendation comprises treating the subject, ceasing an ongoing treatment on the subject, a lifestyle change (e.g., change in dietary habits, exercise, occupation, hobbies, etc), or obtaining further testing for one or more immune-related diseases, disorders, or conditions. In some instances, the methods disclosed herein comprise providing treatment to the subject, stopping treatment of the subject, or conducting testing on the subject (e.g., laboratory or diagnostic tests). The testing can include genetic testing, protein biomarker testing, blood cell testing (e.g., immune cell testing), or immunoglobulin testing (e.g., IgG, IgA, IgM levels).

In some cases, the methods disclosed herein comprise: (a) obtaining an immunological measurement from a biological sample of an individual; (b) applying a machine learning algorithm to the results of the immunological measurement to predict the immune health of the individual; (c) treating the individual for a disease or disorder; and (d) repeating steps (a) and (b) to generate an updated prediction of the immune health of the subject. This approach can be used to evaluate the treatment for efficacy. For example, an improvement in the immune health prediction may be used as an indicator of effective treatment. Conversely, a lack of improvement or a decrease in immune health may indicate an ineffective treatment. In some instances, the immune health prediction for an individual receiving treatment is compared to a baseline or reference such as an individual not receiving treatment (e.g., control). Therefore, the evaluation of the treatment may be based on the changes in immune health relative to the reference. For example, an immune health prediction that remains the same over time during treatment may still indicate effectiveness of the therapy if the reference is a decrease in immune health. Thus, statistically significant similarities and/or differences for an individual in comparison to a baseline or reference can be used to inform a treatment evaluation. In some instances, the immune health of an individual is determined over a plurality of time points to build an immune health timeline. Any of the other metrics disclosed herein can also be monitored over time, for example, antibody or IgG diversity, inflammation score, age, BMI, cytokines, immune cell counts/fractions.

In some cases, the at least one of autoimmune diseases, inflammatory diseases, immunodeficiency diseases, or cancer can be any one of the following: achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis Suppurativa (HS) (Acne Inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, pptic neuritis, palindromic rheumatism (PR), PANDAS, paraneoplastic cerebellar degeneration (PCD), paroxysmal nocturnal hemoglobinuria (PNH), parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada Disease, acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblstoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma [DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor. In some aspects, the cancer type comprises acute lymphoblastic leukemia, acute myeloid leukemia, bladder cancer, breast cancer, brain cancer, cervical cancer, cholangiocarcinoma, colon cancer, colorectal cancer, endometrial cancer, esophageal cancer, gastrointestinal cancer, glioma, glioblastoma, head and neck cancer, kidney cancer, liver cancer, lung cancer, lymphoid neoplasia, melanoma, a myeloid neoplasia, ovarian cancer, pancreatic cancer, pheochromocytoma and paraganglioma, prostate cancer, rectal cancer, squamous cell carcinoma, testicular cancer, stomach cancer, or thyroid cancer.

### Definitions

“Immune health” herein refers to a subject's immune state. Immune health can be predicted from measurements of markers of immune health. Immune health can refer to immunosenescence or immune age. In some instances, a measurement of the immune age of a subject indicates immunosenescence of the subject's immune health.

“Immunological measurement” herein refers to a measurement of one or a combination of two or more markers of immune health.

“Peptides predictive of immune health” herein refers to a set of array peptides that have been identified to bind analytes (e.g. antibodies) that are present in biological samples obtained from a plurality of reference subjects for which markers of immune health are known. For example, in some instances, the state of immune health can be determined by the binding of antibodies to a set of peptides that is characteristic for a group of healthy subjects of a known chronological age. In this instance, the state of immune health can be referred to as immune age. In other instances, the state of immune health can be determined by measurement of BMI and other markers of immune health.

“Marker of immune health” herein refers to measurable or detectable trait known to be associated and/or correlated to a state of immune health. For example, chronological age can be a marker of immune health that is a measureable trait that is associated/correlated with the immunological state of one or more subjects. It is known that increasing chronological age is associated with a decrease in immune health (for example, decreased immunity due to thymic involution, PMID 11531948). Another example of a marker of immune health is BMI, which is a measurable trait that is associated/correlated with the immunological state of one or more subjects. It is known that increasing BMI is associated with increased low grade chronic inflammation, which in turn diminishes immunological health (e.g., C-reactive protein levels are higher, on average, in individuals with higher BMI, PMID 21219177). Other markers of immune health include analytes that are associated with the state of the adaptive, innate or overall immune system e.g. cytokines, which can be associated/correlated with inflammation, and consequent immune health.

“Measurements of at least one marker of immune health” include for example, level of cytokines, signals of antibody binding to a set of array peptides, immune protein or nucleic acid sequence, metabolite level, and blood cell count, or combinations thereof.

“A range of known measurements of at least one marker of immune health” herein refers to measurements of at least one marker of immune health that are made in a reference set of subjects over a range of values for the marker. For example, a range of known measurements of BMI, as the marker, can be made in reference subjects over a range of known chronological age spanning 25 to 100 years of age.

“Immune age” herein refers to the predicted state of subject's immune health that is calculated by an algorithm from measurements of one or more immunological markers that are known to be associated with and/or correlate to the chronological age of healthy reference subjects. By comparing a subject's predicted immune age to the mean immune age of a cohort of healthy reference of known chronological age, the state of the subject's immune system i.e. the immune age of the subject, can be determined to approximate that of individuals of corresponding chronological age, individuals who are older than the subject, or individuals who are younger than the subject. For example, the subject might be 50 years old, but have a predicted immune age value that is typical of people who are 40 years old, suggesting that the subject has greater immune health than most people who are 50 years old.

“Motif” or “submotif” herein refers to a linear sequence of one or more amino acids. The motifs or submotifs described herein typically are in reference to serine and/or threonine amino acid sequences (or combinations thereof) that are between 1-4 amino acids in length and within 0 to 6 amino acids of the N-terminus, and are part of peptide probes such as those located on peptide microarrays. These motifs or submotifs are often characterized by enhanced signal with respect to association with chronological age or indicators thereof, and thus can be useful in predicting immune health such as an immune wellness metric or score.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In some embodiments, the display is a wearable display. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

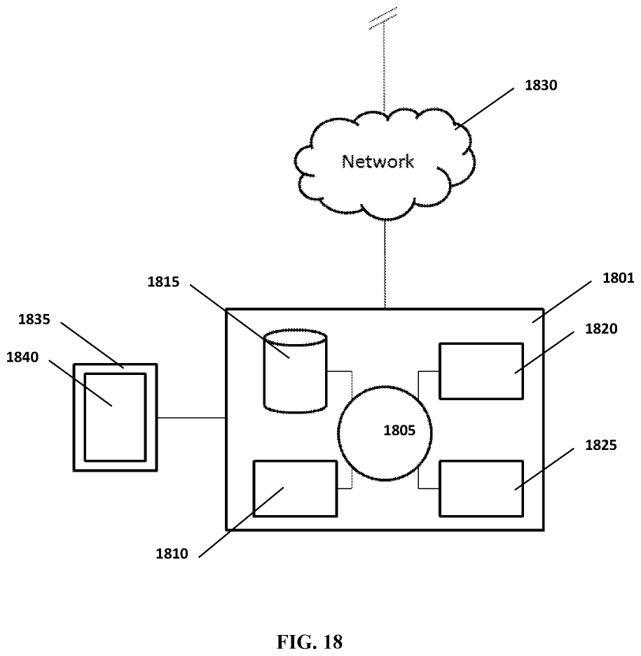

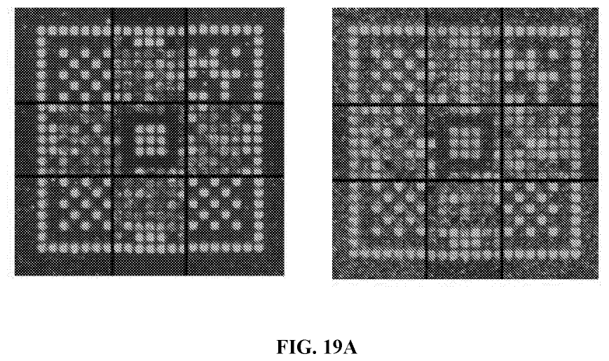

Referring to FIG. 18, in a particular embodiment, an exemplary digital processing device 1801. In this embodiment, the digital processing device 1801 includes a central processing unit (CPU, also “processor” and “computer processor” herein) 1805, which can be a single core or multi core processor, or a plurality of processors for parallel processing. The digital processing device 1801 also includes memory or memory location 1810 (e.g., random-access memory, read-only memory, flash memory), electronic storage unit 1815 (e.g., hard disk), communication interface 1820 (e.g., network adapter) for communicating with one or more other systems, and peripheral devices, such as cache, other memory, data storage and/or electronic display adapters. The memory 1810, storage unit 1815, interface 1820 and peripheral devices are in communication with the CPU 1805 through a communication bus (solid lines), such as a motherboard. The storage unit 1815 can be a data storage unit (or data repository) for storing data. The digital processing device 1801 can be operatively coupled to a computer network (“network”) 1830 with the aid of the communication interface 1820. The network 1830 can be the Internet, an internet and/or extranet, or an intranet and/or extranet that is in communication with the Internet. The network 1830 in some cases is a telecommunication and/or data network. The network 1830 can include one or more computer servers, which can enable distributed computing, such as cloud computing. The network 1830, in some cases with the aid of the device 1801, can implement a peer-to-peer network, which may enable devices coupled to the device 1801 to behave as a client or a server. The digital processing device 1801 can include an output 1835 such as a display or monitor 1840.

Continuing to refer to FIG. 18, the CPU 1805 can execute a sequence of machine-readable instructions, which can be embodied in a program or software having one or more software modules 1825. The instructions may be stored in a memory location, such as the memory 1810. The instructions can be directed to the CPU 1805, which can subsequently program or otherwise configure the CPU 1805 to implement methods of the present disclosure. Examples of operations performed by the CPU 1805 can include fetch, decode, execute, and write back. The CPU 1805 can be part of a circuit, such as an integrated circuit. One or more other components of the device 1801 can be included in the circuit. In some cases, the circuit is an application specific integrated circuit (ASIC) or a field programmable gate array (FPGA).

Continuing to refer to FIG. 18, the storage unit 1815 can store files, such as drivers, libraries and saved programs. The storage unit 1815 can store user data, e.g., user preferences and user programs. The digital processing device 1801 in some cases can include one or more additional data storage units that are external, such as located on a remote server that is in communication through an intranet or the Internet.

Continuing to refer to FIG. 18, the digital processing device 1801 can communicate with one or more remote computer systems through the network 1830. For instance, the device 1801 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the digital processing device 1801, such as, for example, on the memory 1810 or electronic storage unit 1815. The machine executable or machine readable code can be provided in the form of software. During use, the code can be executed by the processor 1805. In some cases, the code can be retrieved from the storage unit 1815 and stored on the memory 1810 for ready access by the processor 1805. In some situations, the electronic storage unit 1815 can be precluded, and machine-executable instructions are stored on memory 1810.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's on or more CPUs, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Apache Hadoop, Microsoft® .NET, or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PUP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of user, patent, phenotypic, genomic, microbiome, and metabolome information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Numbered Embodiments

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A method of measuring immune health in a subject comprising, obtaining an immunological measurement from a biological sample from the subject, wherein the immunological measurement comprises antibody-peptide binding. 2. The method of embodiment 1, wherein the immunological measurement further comprises one or more of the group consisting of: an immune protein sequence, a cytokine level, a metabolite level, and a blood cell count. 3. The method of embodiment 2, wherein the cytokine is selected from TNFα, GM-CSF, MCP-1 (CCL2), MCP-3, IFNα, IFNγ, IL1β, IL2, IL4, IL5, IL6, IL7, IL8, IL10, IL12, IL13, IL17, IL18, IL21, CRP, EGFR, IP10 (CXCL10), Eotaxin (CCL11), MIG, AGP, sTNF-RI, sTNF-RII, sIL2RA, sIL1RA, sIL1RII, sIL6R, CD40L, IL18BP, EGF, VEGF, resistin, leptin, adiponectin, alpha-1-antitrypsin, and free fatty acids. 4. The method of any one of embodiments 2 to 3, wherein the cytokine is selected from CD40L, EGF, Eotaxin (CCL11), GM-CSF, IFNα, IFNγ, IL-1β, sIL-RA, sIL-2R, IL-6, IP-10 (CXCL10), MCP-1 (CCL2), TNFα, sTNF-RI, and sTNF-RII. 5. The method of any one of embodiments 2 to 4, wherein the cytokine is selected from Eotaxin (CCL11), sIL-1RA, sIL-2R, sTNF-RI, IP10 (CXCL10), TNFα, IFNα, IFNγ, IL6, sTNF-RII, and IL-1β. 6. The method of any one of embodiment 2 to 5, wherein cytokine level is measured in a biological fluid. 7. The method of embodiment 6, wherein the biological fluid is selected from the group consisting of serum, whole blood, dried blood, plasma, saliva, and a combination thereof 8. The method of embodiment 6, wherein the cytokine level is measured in a cytokine assay selected from the group consisting of a bead assay, an aptamer assay, an ELISA assay, and an ELISPOT assay. 9. The method of embodiment 1, wherein antibody-peptide binding is measured in a peptide array binding assay, wherein the peptide array binding assay comprises (a) contacting a sample from the subject to a peptide array comprising a plurality of different peptides on distinct features of the array; (b) detecting the binding of antibodies present in the sample to a set of peptides on the peptide array to obtain a pattern of binding signals, wherein the pattern comprises binding signals each associated with a distinct peptide on the array; and (c) comparing the pattern of binding signals in the sample to the pattern of binding signals obtained in reference samples, wherein the binding signals obtained from the binding of the sample correspond to a same set of peptides predictive of immune health identified in a plurality of healthy reference subjects, thereby determining the immune health of the subject. 10. The method of embodiment 9, wherein the sample is a biological fluid. 11. The method of embodiment 10, wherein the biological fluid is selected from the group consisting of whole blood, serum, plasma, saliva, and a combination thereof 12. The method of embodiment 11, wherein blood is dried blood. 13. The method of any one of embodiments 9 to 12, wherein the peptide array is a peptide microarray. 14. The method of any one of embodiments 9 to 13, wherein the peptide array comprises at least about 10,000 distinct peptides. 15. The method of any one of embodiments 9 to 13, wherein the peptide array comprises at least about 3,000,000 distinct peptides. 16. The method of any one of embodiments 9 to 15, wherein the peptide array comprises peptides having 20 or fewer amino acids. 17. The method of any one of embodiments 9 to 15, wherein the peptide array comprises peptides having at least 20 amino acids. 18. The method of any one of embodiments 9 to 17, wherein the peptide array comprises peptides comprising natural amino acids. 19. The method of any one of embodiments 9 to 18, wherein the peptide array comprises peptides comprising unnatural amino acids. 20. The method of any one of embodiments 9 to 19, wherein the peptide array comprises a plurality of peptides characterized by at least one serine motif, threonine motif, serine-threonine motif, or any combination thereof 21. The method of embodiment 20, wherein the serine motif comprises S, SS, SSS, or SSSS. 22. The method of embodiment 20, wherein the threonine motif comprises T or TT. 23. The method of embodiment 20, wherein the serine-threonine motif comprises TS or ST. 24. The method of any one of embodiments 20-23, wherein each of the at least one serine motif, threonine motif, or serine-threonine motif is positioned no more than 1, 2, 3, 4, 5 or 6 amino acids from the N-terminus. 25. The method of any one of embodiments 20-24, wherein the plurality of peptides are acetylated. 26. The method of any one of embodiments 20-25, wherein a machine learning algorithm generates a prediction of immune health based on the immunological measurement. 27. The method of embodiment 26, wherein the machine learning algorithm comprises a panel of peptide features comprising the plurality of peptides characterized by at least one serine motif, threonine motif, serine-threonine motif, or any combination thereof 28. The method of embodiment 26 or 27, wherein the plurality of peptides make up at least 10%, 20%, 30%, 40%, 50%, 60%, 70%, 80%, 90%, or 95% of the panel of peptide features. 29. The method of any one of embodiments 26-28, wherein the plurality of peptides comprises at least 50, 100, 150, 200, 250, 300, or 350 peptides. 30. The method of any one of embodiments 26-29, wherein at least 10%, 20%, 30%, 40%, 50%, 60%, 70%, 80%, 90%, or 95% of the plurality of peptides are statistically correlated with age. 31. The method of embodiment 30, wherein at least 10%, 20%, 30%, 40%, 50%, 60%, 70%, 80%, 90%, or 95% the plurality of peptides that are statistically correlated with age have an N-terminal di-serine (SS) motif. 32. The method of any one of embodiments 9 to 19, wherein the peptide array comprises peptides having a sequence comprising EX1(X2)n (SEQ ID NO: 1), wherein X1 comprises an amino acid selected from A, S, R, Y, and V, X2 comprises any amino acid. 33. The method of embodiment 32, wherein n is between 3 and 30. 34. The method of embodiment 32 or embodiment 33, wherein antibody-peptide binding to a peptide having a sequence of SEQ ID NO: 1 is associated with body mass index (BMI). 35. The method of any one of embodiments 9 to 34, wherein the peptide array comprises peptides having a sequence comprising SS(X)n (SEQ ID NO: 2), wherein X comprises any amino acid. 36. The method of embodiment 35, wherein n is between 3 and 30. 37. The method of embodiment 35, wherein antibody-peptide binding to a peptide having a sequence of SEQ ID NO: 2 is associated with chronological age. 38. The method of any one of embodiments 9 to 37, wherein the plurality of healthy reference subjects are subjects not having an immune altering condition. 39. The method of embodiment 38, wherein the immune altering condition is selected from an autoimmune disease, an inflammatory disease, an immunodeficiency disease, and a cancer. 40. The method of any one of embodiments 9 to 39, wherein the set of peptides predictive of immune health are identified by a method comprising: (i) providing a same peptide array and contacting a plurality of reference samples from a plurality of reference subjects to the peptide array; (ii) detecting the binding of antibodies present in each of the reference samples to the peptides on the array to obtain a pattern of binding signals for each of the reference samples, wherein each pattern of binding signals corresponds to one of a range of known measurements of at least one marker of immune health; (iii) measuring the binding signal associated with each peptide in each of the pattern of binding signals obtained for each of the reference samples; (iii) determining the correlation of the binding signal for each of the peptides in the plurality of reference samples to the range of measurements of the at least one known marker of immune health; and (iv) identifying a set of peptides having a combination of binding signals that correlates to the at least one marker of immune health, thereby identifying the set of peptides predictive of immune health. 41. The method of embodiment 40, wherein range of known measurements is selected for chronological age and body mass index. 42. The method of embodiment 40, wherein step (iv) comprises using a statistical model selected from Elastic Net regression, SVM, and neural networks. 43. The method of embodiment 40, wherein the at least one marker of immune health is selected from chronological age, body mass index, at least one cytokine, and a combination thereof 44. The method of embodiment 40, wherein the at least one marker of immune health further comprises one or more of the group consisting of an immune protein sequence, a cytokine level, a metabolite level, and a blood cell count. 45. The method of any one of embodiments 9 to 44, wherein the immune health of the subject corresponds an immunological measurement that is less than, equal to, or greater than the same immunological measurement obtained in the healthy reference subjects having a chronological age corresponding to the immune age of the subject. 46. The method of embodiment 45, wherein the immune health corresponds to a chronological age that is greater than, equal to, or less than the chronological age of the subject, thereby determining that the immune health of the subject is greater than, equal to, or less than the immune age of healthy reference subjects. 47. The method of embodiment 45, wherein the immune health corresponds to a BMI that is greater than, equal to, or less than the BMI of the subject, thereby determining that the immune health of the subject is greater than, equal to, or less than the immune health of healthy reference subjects. 48. The method of embodiment 45, wherein the immune health corresponds to a combination of chronological age and BMI that is greater than, equal to, or less than the chronological age of the subject, thereby determining that the immune health of the subject is greater than, equal to, or less than the immune health of healthy reference subjects. 49. The method of any one of embodiments 40 to 48, wherein the marker is chronological age, and wherein the set of peptides predictive of immune health comprise at least one of the sequence motifs provided in Table 1. 50. The method of any one of embodiments 40 to 48, wherein the marker is chronological age, and wherein the set of peptides predictive of immune health comprise at least one of the following sequence motifs: S, SS, SSS, SSSS, ST, TS, TT, or TTT. 51. The method of any one of embodiments 40 to 48, wherein the marker is BMI, and wherein the set of peptides predictive of immune health comprise at least one of the sequence motifs provided in Table 2. 52. The method of any one of embodiments 40 to 48, wherein the marker is BMI, and wherein the set of peptides predictive of immune health comprise at least one of the following sequence motifs: S, SS, SSS, SSSS, ST, TS, TT, or TTT. 53. The method of any one of embodiments 2 to 51, wherein the immune protein is selected from the group consisting of an immunoglobulin and a T cell receptor. 54. The method of any one of embodiments 2 to 53, wherein the immune protein sequence is determined by sequencing a nucleic acid encoding the immune protein. 55. The method of any one of embodiments 2 to 54, wherein the metabolite is selected from a fatty acid, an amino acid, a sugar, an enzyme substrate, and combinations thereof. 56. The method of any one of embodiments 2 to 55, wherein the blood cell is selected from one or more of an erythrocyte, a leukocyte, a neutrophil, an eosinophil, a basophil, a lymphocyte, a T cell, a CD4+ T cell, a CD8+ T cell, a regulatory T cell, a γδ T cell, a natural killer cell, a natural killer T cell, a monocyte, a macrophage, and a platelet. 57. A computer-implemented method of predicting an immune health of a subject comprising: a) ingesting, by a computer, results of an immunological measurement from a biological sample from the subject, wherein the immunological measurement comprises antibody-peptide binding; and b) applying, by the computer, a machine learning algorithm to the results of the immunological measurement to predict the immune health of the subject. 58. The method of embodiment 57, further comprising performing, by the computer, feature selection. 59. The method of embodiment 58, wherein the feature selection is performed by t-test, correlation, principal component analysis (PCA), or a combination thereof. 60. The method of embodiment 57, wherein the machine learning algorithm is implemented as: a linear classifier, a neural network, a support vector machine (SVM), an adaptively boosted classifier (AdaBoost), decision tree learning, or a combination thereof 61. The method of embodiment 60, wherein the machine learning algorithm is implemented as a linear classifier, and wherein a linear model is learned by elastic net. 62. The method of embodiment 57, wherein the machine learning algorithm is implemented as ridge regression, lasso regression, regression trees, forward stepwise regression, backward elimination, support vector regression, or a combination thereof 63. The method of embodiment 57, further comprising comparing, by the computer, a proxy measure of the immune health of the subject to the predicted immune health of the subject to determine a residual score. 64. The method of embodiment 63, wherein the proxy measure of the immune health of the subject comprises: chronological age, body mass index (BMI), immune disease or immune disease state, response to treatment in autoimmune disease, response to treatment in immunotherapy, erythrocyte sedimentation rate, antinuclear autoantibodies, rheumatoid factor, fibrinogen, T cell TCR diversity, B cell immunoglobulin diversity, quantification of lymphocytes, quantification of myeloid cells, endogenous steroids, quantification of complement, or a combination thereof 65. The method of embodiment 64, wherein the proxy measure of the immune health of the subject comprises a combination of chronological age and body mass index (BMI). 66. The method of embodiment 57, wherein the predicted immune health is expressed as an immune age. 67. The method of embodiment 57, further comprising ingesting survey data pertaining to the current or past health of the subject, and wherein the machine learning algorithm is further applied to the survey data. 68. The method of embodiment 57, further comprising generating, by the computer, a report. 69. The method of embodiment 68, wherein the report is implemented as a mobile application or a web application. 70. The method of any one of embodiments 57 to 69, wherein the immunological measurement further comprises one or more of the group consisting of: antibody-peptide binding, an immune protein sequence, a cytokine level, a metabolite level, and a blood cell count. 71. The method of embodiment 70, wherein the cytokine is selected from TNFα, GM-CSF, MCP-1 (CCL2), MCP-3, IFNα, IFNγ, IL1β, IL2, IL4, IL5, IL6, IL7, IL8, IL10, IL12, IL13, IL17, IL18, IL21, CRP, EGFR, IP10 (CXCL10), Eotaxin (CCL11), MIG, AGP, sTNF-RI, sTNF-RII, sIL2RA, sIL1RA, sIL1RII, sIL6R, CD40L, IL18BP, EGF, VEGF, resistin, leptin, adiponectin, alpha-1-antitrypsin, and free fatty acids. 72. The method of any one of embodiments 70 to 71, wherein the cytokine is selected from CD40L, EGF, Eotaxin (CCL11), GM-CSF, IFNα, IFNγ, IL-1β, sIL-1RA, sIL-2R, IL-6, IP-10 (CXCL10), MCP-1 (CCL2), TNFα, sTNF-RI, sTNF-RII. 73. The method of any one of embodiments 70 to 71, wherein the cytokine is selected from Eotaxin (CCL11), sIL-1RA, sTL-2R, sTNF-RI, IP10 (CXCL10), TNFα, IFNα, IFNγ, IL6, sTNF-RII, and IL-1β. 74. The method of any one of embodiments 70 to 73, wherein cytokine level is measured in a biological fluid. 75. The method of embodiment 74, wherein the biological fluid is selected from the group consisting of serum, whole blood, dried blood, plasma, saliva, and a combination thereof 76. The method of any one of embodiments 70 to 75, wherein the cytokine level is measured in a cytokine assay selected from the group consisting of a bead assay, an aptamer assay, an ELISA assay, and an ELISPOT assay. 77. The method of any of embodiments 57 to 76, wherein antibody-peptide binding is measured in a peptide array binding assay, wherein the peptide array binding assay comprises (a) contacting a sample from the subject to a peptide array comprising a plurality of different peptides on distinct features of the array; (b) detecting the binding of antibodies present in the sample to a set of peptides on the peptide array to obtain a pattern of binding signals, wherein the pattern comprises binding signals each associated with a distinct peptide on the array; and (c) comparing the pattern of binding signals in the sample to the pattern of binding signals obtained in reference samples, wherein the binding signals obtained from the binding of the sample correspond to a same set of peptides predictive of immune health identified in a plurality of healthy reference subjects, thereby determining the immune health of the subject. 78. The method of embodiment 77, wherein the sample is a biological fluid. 79. The method of embodiment 78, wherein the biological fluid is selected from the group consisting of blood, serum, plasma, saliva, and a combination thereof 80. The method of embodiment 79, wherein blood is dried blood. 81. The method of any one of embodiments 77 to 80, wherein the peptide array is a peptide microarray. 82. The method of any one of embodiments 77 to 80, wherein the peptide array comprises about 10,000 distinct peptides. 83. The method of any one of embodiments 77 to 80, wherein the peptide array comprises about 3,000,000 distinct peptides. 84. The method of any one of embodiments 77 to 83, wherein the peptide array comprises peptides having 20 or fewer amino acids. 85. The method of any one of embodiments 77 to 83, wherein the peptide array comprises peptides having at least 20 amino acids. 86. The method of any one of embodiments 77 to 85, wherein the peptide array comprises peptides comprising natural amino acids. 87. The method of any one of embodiments 77 to 86, wherein the peptide array comprises peptides comprising unnatural amino acids. 88. The method of any one of embodiments 77 to 87, wherein the peptide array comprises a plurality of peptides characterized by at least one serine motif, threonine motif, serine-threonine motif, or any combination thereof. 89. The method of embodiment 88, wherein the serine motif comprises S, SS, SSS, or SSSS. 90. The method of embodiment 88, wherein the threonine motif comprises T or TT. 91. The method of embodiment 88, wherein the serine-threonine motif comprises TS or ST. 92. The method of any one of embodiments 88-91, wherein each of the at least one serine motif, threonine motif, or serine-threonine motif is positioned no more than 1, 2, 3, 4, 5 or 6 amino acids from the N-terminus. 93. The method of any one of embodiments 88-92, wherein the plurality of peptides are acetylated. 94. The method of any one of embodiments 88 to 93, wherein the machine learning algorithm comprises a panel of peptide features comprising the plurality of peptides characterized by at least one serine motif, threonine motif, serine-threonine motif, or any combination thereof. 95. The method of embodiment 94, wherein the plurality of peptides make up at least 10%, 20%, 30%, 40%, 50%, 60%, 70%, 80%, 90%, or 95% of the panel of peptide features. 96. The method of embodiment 94 or 95, wherein the plurality of peptides comprises at least 50, 100, 150, 200, 250, 300, or 350 peptides. 97. The method of embodiment 94 or 96, wherein at least 10%, 20%, 30%, 40%, 50%, 60%, 70%, 80%, 90%, or 95% of the plurality of peptides are statistically correlated with age. 98. The method of embodiment 97, wherein at least 10%, 20%, 30%, 40%, 50%, 60%, 70%, 80%, 90%, or 95% the plurality of peptides that are statistically correlated with age have an N-terminal di-serine (SS) motif. 99. The method of any one of embodiments 77 to 87, wherein the peptide array comprises peptides having a sequence comprising EX1(X2)n (SEQ ID NO: 1), wherein X1 comprises an amino acid selected from A, S, R, Y, and V, X2 comprises any amino acid. 100. The method of embodiment 99, wherein n is between 3 and 30. 101. The method of embodiment 99 or embodiment 100, wherein antibody-peptide binding to a peptide having a sequence of SEQ ID NO: 1 is associated with body mass index (BMI). 102. The method of any one of embodiments 77 to 101, wherein the peptide array comprises peptides having a sequence comprising SS(X)n (SEQ ID NO: 2), wherein X comprises any amino acid. 103. The method of embodiment 102, wherein n is between 3 and 30. 104. The method of embodiment 102, wherein antibody-peptide binding to a peptide having a sequence of SEQ ID NO: 2 is associated with chronological age. 105. The method of any one of embodiments 77 to 104, wherein the plurality of healthy reference subjects are subjects not having an immune altering condition. 106. The method of embodiment 105, wherein the immune altering condition is selected from an autoimmune disease, an inflammatory disease, an immunodeficiency disease, and a cancer. 107. The method of any one of embodiments 77 to 106, wherein the set of peptides predictive of immune health are identified by a method comprising: (i) providing a same peptide array and contacting a plurality of reference samples from a plurality of reference subjects to the peptide array; (ii) detecting the binding of antibodies present in each of the reference samples to the peptides on the array to obtain a pattern of binding signals for each of the reference samples, wherein each pattern of binding signals corresponds to one of a range of known measurements of at least one marker of immune health; (iii) measuring the binding signal associated with each peptide in each of the pattern of binding signals obtained for each of the reference samples; (iii) determining the correlation of the binding signal for each of the peptides in the plurality of reference samples to the range of measurements of the at least one known marker; and (iv) identifying a set of peptides having a combination of binding signals that correlates to the at least one marker of immune health, thereby identifying the set of peptides predictive of immune health. 108. The method of embodiment 107, wherein range of known measurements is selected for chronological age and body mass index. 109. The method of embodiment 107, wherein step (iv) comprises using a statistical model selected from Elastic Net regression, SVM, and neural networks. 110. The method of embodiment 107, wherein the at least one marker of immune health is selected from chronological age, body mass index, at least one cytokine, and a combination thereof. 111. The method of embodiment 107, wherein the at least one marker of immune health further comprises one or more of the group consisting of an immune protein sequence, a cytokine level, a metabolite level, and a blood cell count. 112. The method of any one of embodiments 57 to 111, wherein the immune health of the subject corresponds an immunological measurement that is less than, equal to, or greater than the same immunological measurement obtained in the healthy reference subjects having a chronological age corresponding to the immune health of the subject. 113. The method of embodiment 112, wherein the immune health corresponds to a chronological age that is greater than, equal to, or less than the chronological age of the subject, thereby determining that the immune health of the subject is greater than, equal to, or less than the immune age of healthy reference subjects. 114. The method of embodiment 112, wherein the immune health corresponds to a BMI that is greater than, equal to, or less than the BMI of the subject, thereby determining that the immune health of the subject is greater than, equal to, or less than the immune health of healthy reference subjects. 115. The method of embodiment 112, wherein the immune health corresponds to a combination of chronological age and BMI that is greater than, equal to, or less than the chronological age of the subject, thereby determining that the immune health of the subject is greater than, equal to, or less than the immune health of healthy reference subjects. 116. The method of any one of embodiments 107 to 115, wherein the marker is chronological age, and wherein the set of peptides predictive of immune health comprise at least one of the sequence motifs provided in Table 1. 117. The method of any one of embodiments 107 to 115, wherein the marker is chronological age, and wherein the set of peptides predictive of immune health comprise at least one of the following sequence motifs: S, SS, SSS, SSSS, ST, TS, TT, or TTT. 118. The method of any one of embodiments 107 to 115, wherein the marker is BMI, and wherein the set of peptides predictive of immune health comprise at least one of the sequence motifs provided in Table 2. 119. The method of any one of embodiments 107 to 115, wherein the marker is BMI, and wherein the set of peptides predictive of immune health comprise at least one of the following sequence motifs: S, SS, SSS, SSSS, ST, TS, TT, or TTT. 120. The method of any one of embodiments 1 to 119, further comprising providing a recommendation for the subject based on the measured immune health. 121. The method of embodiment 120, wherein the recommendation comprises providing treatment to the subject, stopping treatment of the subject, adopting a lifestyle change, or obtaining testing for one or more immune-related diseases, disorders, or conditions. 122. The method of any one of embodiments 1 to 119, further comprising providing a therapy or treatment to the subject based on the measured immune health. 123. The method of any one of embodiments 1 to 119, further comprising providing further testing to the subject based on the measured immune health. 124. The method of embodiment 123, wherein the further testing comprises genetic testing, metabolite testing, serum protein testing, blood cell count testing, immunoglobulin testing, or any combination thereof. 125. A computer system comprising a processor and non-transitory computer readable storage medium encoded with a computer program that causes the processor to perform the method of any one of embodiments 57-121.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Immunosignatures Predict Chronological Age and BMI as Proxy Measures of Immune Health

Immunosignatures were tested first for their association with chronological age and with body mass index (BMI) parameters, which were chosen as proxy measures of immune wellness. The immunosignature associations were subsequently used alone or in combination with measured cytokine levels to provide an Immune Index score as a predictor of Immune Health.

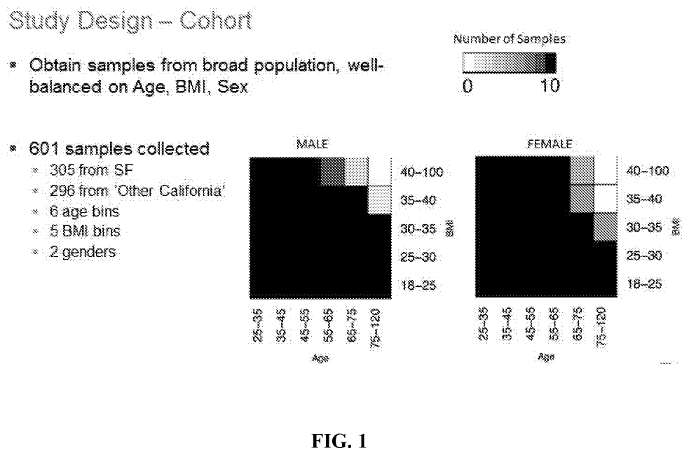

Cohorts: Serum samples from two cohorts of human subjects were obtained from Creative Testing Solutions (CTS) and recruitment was explicitly balanced on age, BMI, and sex. The first cohort (feasibility cohort) consisted of 601 samples that were obtained from healthy subjects at various sites in California. The 601 samples comprised 305 samples from San Francisco (SF), and 296 samples from ‘Other California’ sites (OC). The second cohort (validation cohort) (Val) consisted of 1074 samples that were obtained from healthy subjects at various CTS collection sites in USA. In addition to the serum sample, data on human subjects included collection site, age, sex, ethnicity, height, weight, BMI. Samples were binned by age groups: 25-35, 35-45, 45-55, 55-65, and 75-120 years; and by BMI score groups: 18-25, 25-30, 30-35, 35-40, and 40-100 (e.g. FIG. 1), and determined to be balanced on age, BMI, and sex.

Immunosignature assay: In the immunosignature assays, binding of serum antibodies to each array feature was measured by quantifying fluorescent signal. Arrays displaying a total of 131,712 peptides (V13) (median length of 9 amino acids) at features of 14 μm×14 μm each were utilized to query antibody-binding events. The array layout included 126,009 library-peptide features and 6203 control-peptide features attached to the surface via a common linker. Production quality manufactured microarrays were obtained and rehydrated prior to use by soaking with gentle agitation in distilled water for 1 h, PBS for 20 min and primary incubation buffer (PBST, 1% mannitol) for 1 h. Slides were loaded into an ArrayIt microarray cassette (ArrayIt, Sunnyvale, Calif.) to adapt the individual microarrays to a microtiter plate footprint. Using a Bravo liquid handler (Agilent, USA), 90 μl of each sample was prepared at a 1:625 dilution in primary incubation buffer (PBST, 1% mannitol) and then transferred to the cassette. This mixture was incubated on the arrays for 1 h at 37° C. with mixing on a TeleShake95 (INHECO, Martinsried, Germany) to drive antibody-peptide binding. Following incubation, the cassette was washed 3× in PBST using a BioTek 405TS (BioTek, Winooski, Vt.). Bound antibody was detected using 4.0 nM goat anti-human IgG (H+L) conjugated to AlexaFluor 555 (Thermo-Invitrogen, Carlsbad, Calif.), or 4.0 nM goat anti-human IgA conjugated to DyLight 550 (Novus Biologicals, Littleton, Colo.) in secondary incubation buffer (0.75% casein in PBST) for 1 h with mixing on a TeleShake95 platform mixer, at 37° C. Following incubation with secondary, the slides were again washed with PBST followed by distilled water, removed from the cassette, sprayed with isopropanol and centrifuged dry. Quantitative signal measurements were obtained by determining a relative fluorescent value for each addressable peptide feature.

Data Acquisition. Assayed microarrays were imaged using the Molecular Devices (CA, USA) ImageXpress Micro XLS imager with a Lumencor SOLA solid state white light engine and a Semrock TRITC-B filter cube. The Mapix software application (version 7.2.1) identified regions of the images associated with each peptide feature using an automated gridding algorithm. Median pixel intensities for each peptide feature were saved as a tab-delimitated text file and stored in a database for analysis.

Immunosignature Data Analysis. Fluorescent intensity was measured at each array peptide feature for each of the samples, and used to develop a classifier. Intensity values were log₁₀ transformed after adding a constant value of 100 to improve homoscedasticity. Values were then median normalized for each array by calculating the median value and subtracting it (this is equivalent to log ratio of values before they were log transformed).

Regression models of chronological age, of BMI, and combinations of age and BMI were employed and trained using the Elastic Net Feature selection (see, e.g., Zou, Hui; Hastie, Trevor (2005). “Regularization and Variable Selection via the Elastic Net”. Journal of the Royal Statistical Society, Series B: 301-320; Hastie, Tibshirani and Friedman, The Elements of Statistical Learning, 2^{nd} ed. (2008)) procedure to constrain model complexity. The Elastic Net approach applies Ridge Regression and LASSO penalties, where correlated features tend to be removed as groups. Briefly, Ridge Regression constrains the sum of coefficients to reduce overfit while reducing magnitude of coefficients, but does not eliminate features. The LASSO approach adds a quadratic term that leads to feature selection, but feature selection is unstable when features are correlated. 16- to 32-fold cross validation was used to correct for overfit. see Frank E. Harrell, Jr., Regression Modelling Strategies, Springer Science+Business Media Inc. (2001). Peptides that correlated with age, and with BMI were identified by Pearson correlation.

Cytokine assays: Simultaneous detection and quantification of multiple cytokines were determined using the Luminex xMAP (multi-analyte profiling) technology (ThermoFisher, USA). 25 ul of serum from each of the samples was assayed according to the manufacturer's instructions. The cytokine panel for the feasibility cohort (601 samples) was run as a customized 15-plex: CD40L, EGF, Eotaxin (CCL11), GM-CSF, IFN alpha, IFN gamma, IL-1 beta, IL-RA, IL-2R, IL-6, IP-10 (CXCL10), MCP-1 (CCL2), TNF alpha, TNF-RI, TNF-RII, and hsCRP ran at an additional dilution. Of these markers, the following were incorporated into a model for age: CD40L, EGF, Eotaxin (CCL11), GM-CSF, IFN gamma, IL-1 beta, IL-2R, IL-6, IP-10 (CXCL10), TNF alpha, TNF-RI, TNF-RII. The following markers were incorporated into a model for BMI: EGF, Eotaxin (CCL11), GM-CSF, IL-1 beta, IL-1RA, IL-6, hsCRP.

The cytokine panel for the feasibility cohort (1074 samples) was also run as a customized 11-plex: Eotaxin, sIL1Ra, sIL2Ra, sTNFR1, IP10, TNFα, IFNα, IFNγ, IL6, sTNFR2, IL1beta. C-reactive protein (CRP) was quantified in samples of both cohorts in a simplex assay.

Cytokine association analysis: the level of each of cytokines indicated for each of the two cohorts was measured as described in Example 1, and the elastic net regression model was learned as alpha=0.5 and broad lambda path (10\hat{ }⁵ to 10⁻³ in log-spaced decrements). Parameters were chosen by 16-random subsamplings on training set of 295 samples (80% trained, 20% used for parameter selection in each random subsampling). Training set was collected in California (but outside San Francisco). After model was learned and parameters selected, model was tested on 301 samples collected in San Francisco. Age and BMI correlation with cytokine model was r=0.423 and r=0.418 (respectively).

Results:

Immunosignature Predicts Chronological Age and BMI

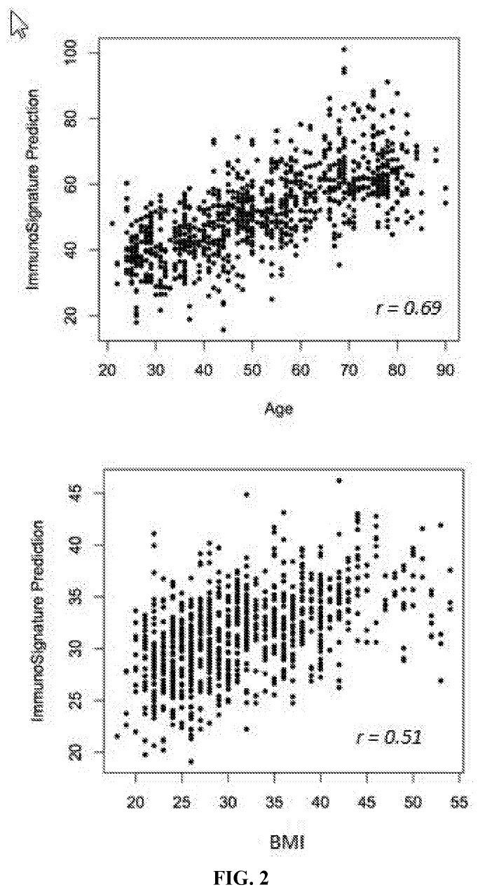

Elastic net model was developed first from immunosignature data obtained from the cohort of 601 samples. The model was trained on 90% of data, with 10% randomly held out on n=16 bootstraps for assessing accuracy. Accuracy on the 10% held out was consistently near r=0.70, with optimal accuracy at selected parameters (alpha=0.01, lambda=1). Accuracy on the 10% held-out was determined to be r=0.69 for correlating immunosignature signal with chronological age (FIG. 2, left panel), with optimal accuracy at selected parameter, and r=0.51 for correlating immunosignature signal with BMI, with optimal accuracy at selected parameter (FIG. 2, right panel).

Verification of the results obtained using the 601 samples cohort was confirmed using the 1074 validation samples, where comparable high correlation with chronological age (r=0.73), and with BMI (r=0.44) was determined. The model was trained on the 601 training samples, using 80% of data, with 20% randomly held out on n=16 bootstraps for assessing accuracy and parameter selection of alpha=0.01 and lambda=1. The 1074 samples were used as independent validation set.

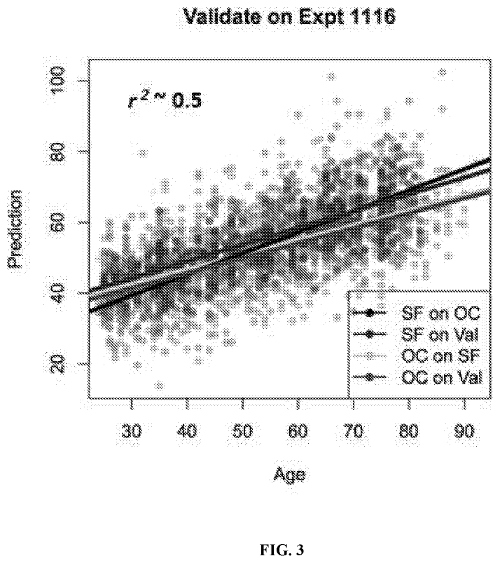

The high accuracy of the regression model was further demonstrated by linear regressions calculated for different training sets having absolute value and dynamic range that were similar in the cohorts (FIG. 3). FIG. 3 shows the correlation between the ImmunoSignature prediction with chronological age determined in separate models developed by: training on samples from San Francisco (SF) and testing on samples collected from non-San Francisco Other California (OC) sites, shown as “SF on OC”; training on samples from San Francisco (SF) and testing on samples collected from the independent validation set of 1074 samples (Val), shown as “SF on Val”; training on samples from non-San Francisco Other California (OC) and testing on samples collected from San Francisco (SF), shown as “OC on SF”; and training on non-San Francisco Other California (OC) sites and testing on the 1074 samples of the validation set (Val), shown as “OC on Val”.

These data show that immunosignature data correlate with chronological age and with BMI, and can therefore be used to predict the immune health of a subject. Additionally, the absolute value and dynamic of the regression output for different cohorts, demonstrates the high accuracy of the regression model.

Furthermore, for each donor, the residual with respect to age was not random and was reproducible across independent mutually exclusive training sets, which suggests that donor-specific chronological age vs immunological age has greater signal than the technical variation and statistical error (which is an important technical validation of using immune health proxy).

### Example 2. Cytokines Levels as Measurements of Markers of Immune Health

Cytokine Levels Correlate with Chronological Age and with BMI

Association of cytokine levels with chronological age, and with BMI were also determined. The level of each of the cytokines defined for the feasibility and the validation cohort was measured by Luminex® beads (Thermo Fisher Scientific, USA), and a linear model was learned by Elastic Net as described for the immunosignature models. Levels of cytokines were measured in samples form the feasibility and the validation cohorts as described in Example 2.

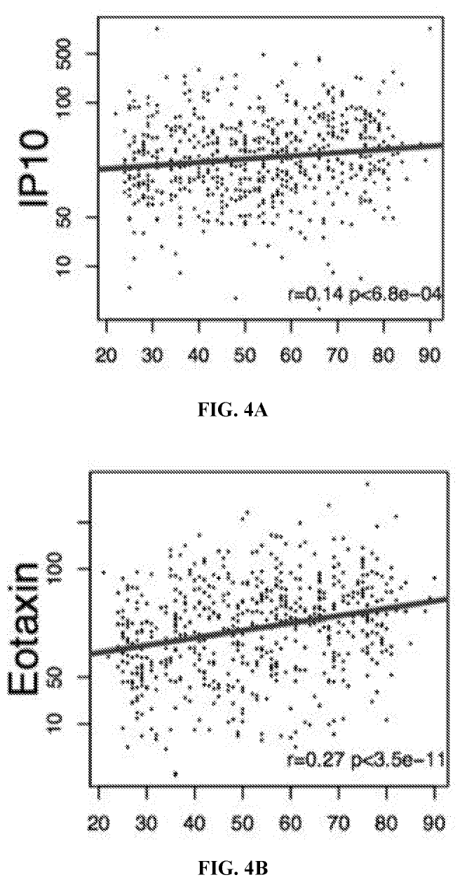

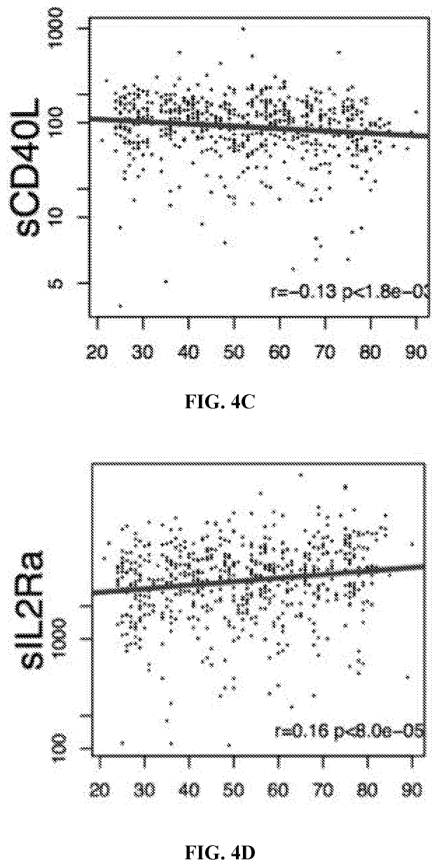

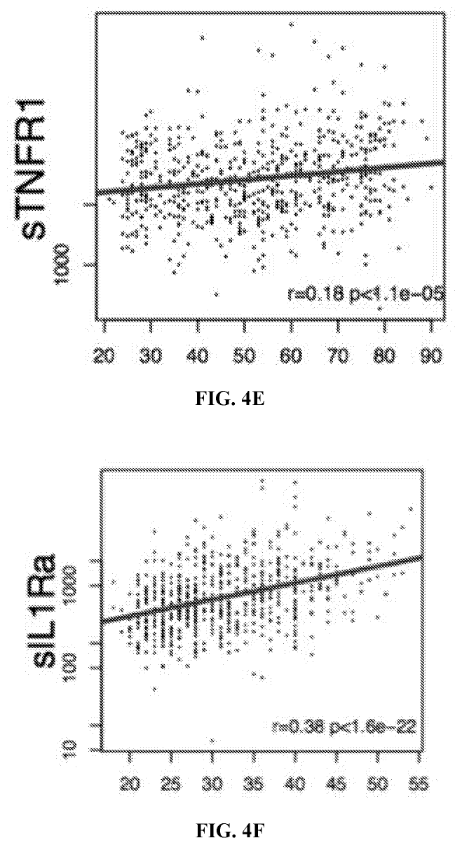

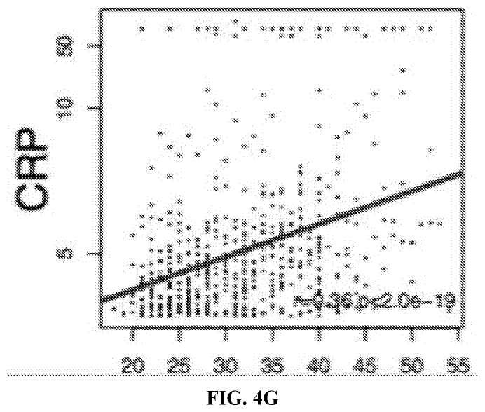

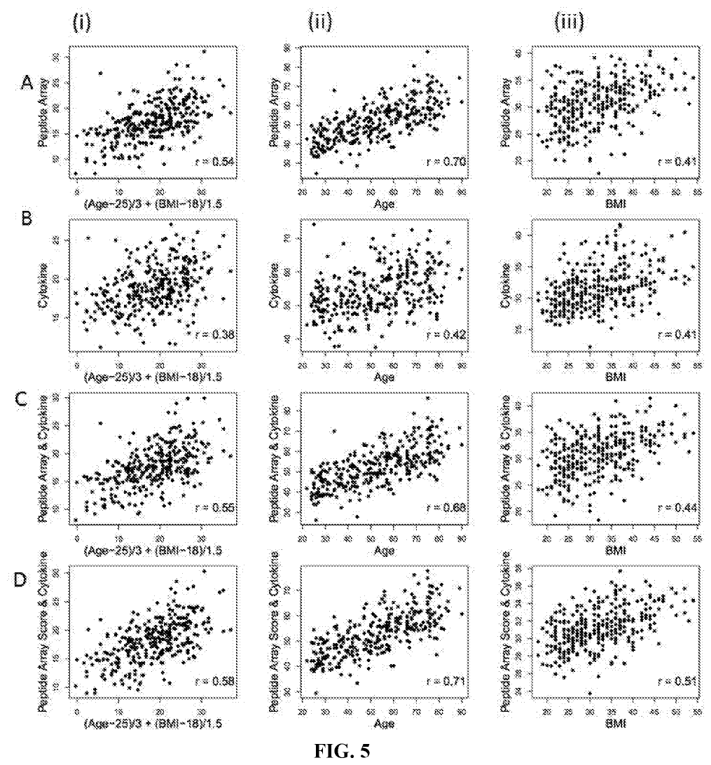

Examples of the association of cytokine levels with age and with BMI are shown in FIG. 4A-FIG. 4G. FIG. 4A plots the relationship between IP10 and age (r=0.14). FIG. 4B plots the relationship between Eotaxin and age (r=0.27). FIG. 4C plots the relationship between sCD40L and age (r=0.13). FIG. 4D plots the relationship between sIL2Ra and age (r=0.16). FIG. 4E plots the relationship between sTNFR1 and age (r=0.18). FIG. 4F plots the relationship between sIL1Ra and BMI (r=0.38). FIG. 4G plots the relationship between CRP and BMI (r=0.36).

### Example 3. Prediction Tasks

Immunosignatures, cytokines, and combination of immunosignatures and cytokines were tested for correlation with chronological age, BMI, and a combination of chronological age and BMI as proxies of immune health (FIG. 5).

Samples of subjects from the feasibility cohort were used in this study.

Elastic net models were developed to determine the association of immunosignature signal data (Row A), cytokine levels measured by ELISA (row B), and combinations of immunosignature signal data and cytokine levels (rows C and D), with known immunological measurements of a function of age and BMI, F(age, BMI), shown as (age-25)/3+(BMI-18/1.5) (see column (i)), chronological age (see column (ii)), and BMI (see column (iii)). F(Age, BMI) was derived to combine different ranges of 25-80 for age and 18-50 for BMI to project values in a single dynamic range that would be equally weighted. The prediction using the combination of immunosignature signal data and cytokine levels shown in row C is derived from signal data obtained from the binding of antibodies in reference samples to all the peptides of the array. The prediction using the combination of immunosignature signal data indicated by “Peptide Array Score” and cytokine levels shown in row C is derived from signal data obtained from the binding of antibodies in reference samples to peptides previously determined to be predictive i.e. peptides that showed association in predictions in row A. The data were obtained according to the immunosignature and cytokine measurements of samples from the feasibility cohort as described in Example 1. The models were trained on data of 90% of the OC samples and tested on data from 10% hold out of the 296 OC samples over 16 or 32 permutations, then tested on the data obtained from the 305 SF samples.

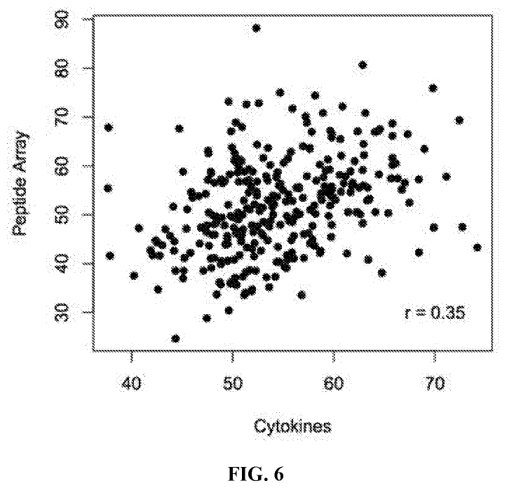

FIG. 5 shows graphs showing the relationship between immunosignatures (e.g. peptide array signals) (row (A)), cytokines (row (B)), and combinations of immunosignatures and cytokines (rows (C) and (D)) and plotting against a combination function of chronological age and BMI (column (i)), chronological age (column (ii)) and BMI (column (iii)), as proxies for immune health. Row (C) trained on example matrix where peptide array and cytokine data were concatenated, whereas row (D) trained on matrix where only score derived from peptide array data was concatenated. The results shown in FIG. 5 demonstrate that known measurements of chronological age, BMI and the combination of chronological age and BMI as can be used to determine immune health when measured by immunosignature assay and/or by cytokine levels. FIG. 6 shows that Immunosignature scores determined relative to chronological age (shown on y-axis as “peptide array”) were shown to be correlated to immunosignature scores determined relative to cytokine levels (shown on x-axis as “Cytokines”). While there is statistically significant correlation (Pearson's correlation coefficient of r=0.35), the majority of signal is independent. This suggests that a multivariate index that uses scores based on peptide array binding and cytokines may be beneficial. This index may present independent data reported jointly as single combined score or as set of scores that represent distinct aspects of the immune health.

### Example 4. Immunosignature Peptides Indicative of Chronological Age and BMI

The linear model developed to predict chronological age using immunosignature data identified a first set of array peptides that have a strong association with chronological age as defined by t-test values p<0.01 and log₁₀ ratio >0.2. Additionally, the regression model identified a second set of peptides from the immunosignature data that were predictive of BMI. Analysis of the peptide sequences for the presence of submotifs revealed that the peptides predictive of age and peptides predictive of BMI comprised the submotifs listed in Tables 1 and 2, respectively.

Tables 1 and 2 show the submotifs and amino acids that were enriched in the significant predictive peptides in the study.

TABLE 1
Sequence submotifs identified in immune health predictive
peptides correlating with chronological age

| type | motif | n | n.lib | enrich | p |
|---|---|---|---|---|---|
| XX | SS | 74 | 2656 | 38.34169144 | 1.70E−105 |
| X | S | 170 | 81047 | 4.452213596 | 7.58E−68 |
| XXX | SSV | 21 | 94 | 59.07414159 | 1.06E−50 |
| X . . . X | S . . . D | 31 | 3735 | 5.597420393 | 1.02E−32 |
| X . . . X | S . . . G | 28 | 4142 | 3.214358175 | 2.88E−30 |
| X . . . X | S . . . G | 30 | 4469 | 4.527179748 | 5.02E−29 |
| XXX | SSA | 13 | 133 | 25.84625887 | 7.27E−27 |
| X . . . X | S . . . D | 22 | 3270 | 3.199051707 | 9.07E−24 |
| X.X | S.V | 25 | 3000 | 10.69646206 | 9.78E−24 |
| XXX | SSL | 11 | 105 | 27.70188771 | 2.76E−23 |
| X . . . X | S . . . D | 24 | 4094 | 5.415577307 | 1.09E−20 |
| X . . . X | S . . . E | 22 | 3435 | 5.916669297 | 7.51E−20 |
| XX | SV | 21 | 2691 | 10.73923178 | 3.03E−19 |
| X.X | S.A | 20 | 2600 | 9.873657289 | 1.39E−18 |
| X . . . X | S . . . E | 19 | 3059 | 4.188812897 | 4.53E−18 |
| XXX | SSF | 8 | 92 | 22.99366174 | 1.27E−16 |
| X . . . X | S . . . S | 17 | 3109 | 5.051374362 | 1.64E−14 |
| X.X | S.L | 17 | 3096 | 7.048056398 | 1.17E−13 |
| XXX | SSY | 7 | 134 | 13.81335649 | 4.04E−13 |
| X . . . X | S . . . E | 13 | 2629 | 2.351251566 | 9.73E−13 |
| X . . . X | S . . . A | 13 | 2075 | 7.039206325 | 1.01E−11 |
| X . . . X | S . . . S | 13 | 2670 | 3.283590035 | 1.95E−11 |
| X . . . X | S . . . E | 15 | 3938 | 4.279706545 | 2.46E−10 |
| X.X | S.F | 14 | 3282 | 5.475337071 | 4.46E−10 |
| X . . . X | S . . . D | 15 | 4442 | 3.794120751 | 1.23E−09 |
| X . . . X | S . . . F | 13 | 3105 | 4.704139493 | 1.29E−09 |
| X . . . X | S . . . G | 15 | 4832 | 2.867779063 | 1.37E−09 |
| X . . . X | S . . . F | 12 | 2765 | 4.009289963 | 1.68E−09 |
| X . . . X | S . . . P | 11 | 2048 | 6.034776306 | 1.96E−09 |
| XXXX | SSVG | 3 | 7 | 11.70669643 | 2.64E−09 |
| XX | SA | 13 | 3233 | 5.533568195 | 5.74E−09 |
| X . . . X | S . . . F | 10 | 2334 | 2.88945534 | 1.44E−08 |
| XXXX | SSAW | 3 | 15 | 5.463125 | 3.42E−08 |
| X.X | S.G | 15 | 5582 | 3.449235348 | 4.68E−08 |
| XX | SL | 11 | 2628 | 5.760165253 | 5.75E−08 |
| X . . . X | S . . . F | 8 | 1804 | 2.108627108 | 6.23E−08 |
| XXX | SSS | 4 | 91 | 11.62316967 | 9.93E−08 |
| X.X | S.Y | 10 | 2549 | 5.035603953 | 3.00E−07 |
| XXXX | SSAA | 2 | 3 | 18.21041667 | 5.37E−07 |
| XXXX | SSLP | 2 | 3 | 18.21041667 | 5.37E−07 |
| XXXX | SSVH | 2 | 3 | 18.21041667 | 5.37E−07 |
| XXXX | SSVR | 2 | 3 | 18.21041667 | 5.37E−07 |
| X . . . X | S . . . L | 10 | 2884 | 3.895858617 | 5.83E−07 |
| X . . . X | S . . . G | 13 | 5356 | 2.727101032 | 6.52E−07 |
| X . . . X | S . . . L | 9 | 2489 | 3.340403801 | 8.47E−07 |
| XX | DG | 25 | 18500 | 1.85967006 | 1.79E−06 |
| XXXX | SSAL | 2 | 5 | 10.92625 | 1.79E−06 |
| XXXX | SSFP | 2 | 6 | 9.105208333 | 2.68E−06 |
| XXX | EDG | 7 | 1341 | 1.38030557 | 2.93E−06 |
| X . . . X | S . . . Y | 8 | 2051 | 4.382508532 | 3.40E−06 |
| XXXX | SSVE | 2 | 8 | 6.82890625 | 5.01E−06 |
| XXXX | SSVD | 2 | 9 | 6.070138889 | 6.44E−06 |
| XX | ED | 21 | 14979 | 1.929319229 | 7.29E−06 |
| X.X | S.R | 9 | 2912 | 3.967094446 | 7.92E−06 |
| XXXX | SSAV | 2 | 10 | 5.463125 | 8.04E−06 |
| X . . . X | S . . . R | 8 | 2427 | 3.70355377 | 1.14E−05 |
| XXXX | SSVF | 2 | 12 | 4.552604167 | 1.18E−05 |
| XXXX | SSYA | 2 | 13 | 4.202403846 | 1.39E−05 |
| XXX | SSR | 3 | 105 | 7.555060285 | 1.58E−05 |
| X . . . X | S . . . S | 9 | 3465 | 2.918352273 | 2.09E−05 |
| X . . . X | S . . . P | 6 | 1458 | 3.801675839 | 2.92E−05 |
| XXXX | SSLF | 2 | 19 | 2.875328947 | 3.05E−05 |
| X . . . X | S . . . W | 7 | 2034 | 3.866745022 | 3.08E−05 |
| XX | SF | 8 | 2613 | 4.213259377 | 3.45E−05 |
| X . . . X | S . . . N | 7 | 2140 | 3.675214661 | 4.23E−05 |
| X . . . X | S . . . A | 6 | 1594 | 3.477317047 | 4.76E−05 |
| X . . . X | S . . . N | 6 | 1784 | 3.106974985 | 8.79E−05 |
| X.X | S.P | 7 | 2428 | 3.700588193 | 0.000124121 |
| X . . . X | S . . . H | 7 | 2586 | 3.041360934 | 0.000135537 |
| XXX | SVG | 3 | 231 | 3.434118311 | 0.000163422 |
| X.X | S.D | 9 | 4537 | 2.546215347 | 0.000227366 |
| X . . . X | S . . . H | 6 | 2141 | 2.588903958 | 0.000233454 |
| X.X | S.W | 7 | 2709 | 3.316732423 | 0.000240181 |
| XXX | SAW | 3 | 286 | 2.773710944 | 0.000305306 |
| XXX | AED | 4 | 740 | 1.42933573 | 0.000365968 |
| X . . . X | S . . . S | 5 | 2104 | 1.129979593 | 0.000371392 |
| X . . . X | S . . . V | 6 | 2350 | 2.358656755 | 0.000380965 |
| X . . . X | S . . . Y | 5 | 1521 | 3.036841647 | 0.000381604 |
| X . . . X | S . . . V | 5 | 1851 | 1.821714955 | 0.000545292 |
| XXX | SVH | 2 | 92 | 5.748415434 | 0.000802006 |
| X . . . X | L . . . G | 8 | 4938 | 1.496650027 | 0.000841847 |
| X.X | S.S | 7 | 3385 | 2.65436577 | 0.000884639 |
| X . . . X | S . . . N | 4 | 1227 | 2.198529344 | 0.001002918 |
| XXX | SVR | 2 | 105 | 5.036706857 | 0.001042079 |
| XXX | SSQ | 2 | 106 | 4.989190754 | 0.001061805 |
| XXX | SSH | 2 | 110 | 4.807765636 | 0.001142497 |
| XX | SY | 7 | 3445 | 2.796252804 | 0.001193397 |
| X . . . X | S . . . V | 6 | 2749 | 2.452307657 | 0.001230031 |
| X . . . X | S . . . N | 3 | 721 | 1.978482994 | 0.001233388 |
| XXX | SAA | 2 | 116 | 4.559088103 | 0.001268882 |
| X . . . X | V . . . D | 7 | 4149 | 1.558604628 | 0.001411062 |
| XXX | SLP | 2 | 124 | 4.264953387 | 0.001447323 |
| XXX | SSW | 2 | 125 | 4.23083376 | 0.001470422 |
| X.X | V.D | 9 | 6165 | 1.87383277 | 0.001911581 |
| XXX | SFP | 2 | 153 | 3.456563529 | 0.002188032 |
| XXX | NFS | 3 | 586 | 1.353722406 | 0.002391085 |
| XXX | SAL | 2 | 192 | 2.754449062 | 0.003410806 |
| X . . . X | S . . . W | 4 | 1677 | 2.203475799 | 0.004647026 |
| X . . . X | S . . . P | 3 | 951 | 2.12744125 | 0.004824724 |
| X . . . X | S . . . Q | 4 | 1814 | 2.037061144 | 0.006099589 |
| X . . . X | S . . . W | 3 | 1139 | 1.776292036 | 0.007896193 |
| X . . . X | S . . . Q | 3 | 1333 | 1.517776916 | 0.012039273 |
| X . . . X | V . . . G | 6 | 4994 | 1.109900555 | 0.014753254 |
| X . . . X | S . . . H | 3 | 1652 | 1.224695296 | 0.021110928 |
| X . . . X | S . . . Q | 2 | 780 | 1.219219007 | 0.021464883 |
| X . . . X | S . . . K | 2 | 888 | 1.070935615 | 0.027261733 |

TABLE 2
Sequence submotifs identified in immune health
predictive peptides correlating with BMI

| type | motif | n | n.lib | enrich | p |
|---|---|---|---|---|---|
| XX | EA | 111 | 3464 | 11.63943988 | 5.27E−82 |
| X | E | 443 | 88052 | 1.986694539 | 1.02E−50 |
| X . . . X | E . . . K | 44 | 885 | 16.66731747 | 4.09E−43 |
| X . . . X | E . . . K | 45 | 1387 | 11.52675374 | 2.50E−35 |
| X.X | E.A | 54 | 2614 | 7.714856385 | 2.70E−31 |
| XX | ES | 51 | 2706 | 6.845881379 | 1.13E−27 |
| X . . . X | E . . . K | 39 | 1953 | 7.45387967 | 6.89E−23 |
| X | A | 263 | 57190 | 1.815943151 | 1.40E−22 |
| X . . . X | E . . . Q | 37 | 1850 | 7.465347177 | 8.25E−22 |
| XX | ER | 42 | 2742 | 5.56376561 | 8.70E−20 |
| XXX | EAA | 14 | 115 | 19.44361247 | 1.02E−19 |
| X . . . X | E . . . Q | 24 | 794 | 10.13320996 | 3.75E−19 |
| X . . . X | E . . . R | 38 | 2408 | 5.874587823 | 1.00E−18 |
| X . . . X | E . . . P | 30 | 1460 | 7.669877237 | 2.83E−18 |
| X . . . X | E . . . N | 22 | 696 | 10.59667813 | 4.45E−18 |
| X . . . X | E . . . N | 27 | 1201 | 7.987147763 | 8.93E−18 |
| X . . . X | E . . . A | 30 | 1605 | 6.976959979 | 3.54E−17 |
| X . . . X | E . . . K | 35 | 2320 | 5.616041989 | 8.83E−17 |
| XXX | EAS | 14 | 195 | 11.46674581 | 1.94E−16 |
| X . . . X | E . . . N | 30 | 1736 | 6.450472791 | 2.79E−16 |
| X . . . X | E . . . A | 24 | 1038 | 8.214570296 | 3.15E−16 |
| X . . . X | E . . . G | 43 | 4056 | 3.554076825 | 4.53E−16 |
| X . . . X | E . . . S | 31 | 2113 | 4.918339445 | 1.49E−15 |
| XXX | EAV | 14 | 227 | 9.850288253 | 1.62E−15 |
| XXX | ESA | 12 | 142 | 13.49707505 | 3.77E−15 |
| X . . . X | E . . . S | 34 | 2631 | 4.591229806 | 6.82E−15 |
| X.X | E.V | 37 | 3071 | 4.499472242 | 1.60E−14 |
| X . . . X | E . . . Q | 24 | 1286 | 6.630422991 | 3.21E−14 |
| X . . . X | E . . . A | 30 | 2064 | 5.410804574 | 3.50E−14 |
| X . . . X | E . . . P | 30 | 2078 | 5.374350645 | 4.15E−14 |
| X . . . X | E . . . P | 17 | 554 | 10.28715915 | 4.36E−14 |
| X.X | E.S | 37 | 3381 | 4.086920809 | 2.70E−13 |
| X.X | A.K | 35 | 3036 | 4.305325054 | 2.79E−13 |
| X . . . X | E . . . R | 24 | 1505 | 5.66559732 | 8.75E−13 |
| X . . . X | E . . . S | 34 | 3164 | 4.011090455 | 2.02E−12 |
| X . . . X | A . . . G | 40 | 4530 | 3.137131702 | 4.51E−12 |
| X . . . X | E . . . G | 40 | 4541 | 3.129532396 | 4.85E−12 |
| X . . . X | E . . . N | 27 | 2176 | 4.619076552 | 2.26E−11 |
| X . . . X | E . . . S | 34 | 3496 | 3.6204102 | 4.14E−11 |
| X . . . X | E . . . Y | 26 | 2083 | 4.646590121 | 4.64E−11 |
| X . . . X | E . . . P | 18 | 968 | 6.606449355 | 5.35E−11 |
| XX | AQ | 39 | 4486 | 3.157856012 | 6.95E−11 |
| X.X | E.Q | 30 | 2831 | 3.957501195 | 9.97E−11 |
| XX | EV | 29 | 2665 | 3.952644633 | 1.33E−10 |
| X | S | 280 | 81047 | 1.364230278 | 4.44E−10 |
| XXX | EAH | 10 | 226 | 7.067052571 | 4.52E−10 |
| X . . . X | E . . . R | 17 | 1020 | 5.58733938 | 5.66E−10 |
| X . . . X | E . . . V | 18 | 1217 | 4.958361981 | 1.15E−09 |
| X.X | E.R | 29 | 2959 | 3.6600979 | 1.16E−09 |
| XXX | EAR | 10 | 257 | 6.214606541 | 1.56E−09 |
| X . . . X | E . . . Y | 16 | 962 | 5.909025618 | 2.99E−09 |
| X . . . X | E . . . Q | 24 | 2229 | 4.008219162 | 4.38E−09 |
| X.X | E.P | 25 | 2420 | 3.858018555 | 5.75E−09 |
| XXX | ESQ | 7 | 110 | 10.16370652 | 1.55E−08 |
| X . . . X | A . . . S | 31 | 3816 | 3.02415199 | 1.81E−08 |
| XX | QK | 39 | 5549 | 2.552918015 | 2.18E−08 |
| XXX | ERG | 8 | 192 | 6.654807838 | 3.99E−08 |
| X . . . X | E . . . G | 37 | 5358 | 2.570687593 | 4.79E−08 |
| X . . . X | A . . . S | 27 | 3196 | 3.153385072 | 5.16E−08 |
| X . . . X | A . . . Q | 23 | 2378 | 3.600528662 | 6.17E−08 |
| X . . . X | A . . . K | 17 | 1394 | 4.332684943 | 8.49E−08 |
| X . . . X | E . . . D | 26 | 3289 | 2.650121445 | 1.05E−07 |
| XXX | ERS | 8 | 219 | 5.834352077 | 1.10E−07 |
| XXXX | KGYN | 4 | 18 | 4.556861349 | 1.12E−07 |
| X.X | A.A | 23 | 2444 | 3.514522304 | 1.19E−07 |
| XXX | AQK | 10 | 409 | 3.905021714 | 1.23E−07 |
| X . . . X | E . . . L | 17 | 1507 | 3.781742646 | 1.56E−07 |
| X . . . X | A . . . G | 27 | 3596 | 2.517099498 | 1.63E−07 |
| XX | EQ | 25 | 2874 | 3.159659116 | 1.69E−07 |
| X.X | E.G | 33 | 4727 | 2.607161936 | 2.51E−07 |
| X . . . X | E . . . V | 23 | 2721 | 3.146658272 | 6.21E−07 |
| XXX | ERA | 6 | 118 | 8.121121429 | 6.32E−07 |
| XX | NK | 34 | 5147 | 2.399450167 | 6.73E−07 |
| X . . . X | A . . . K | 21 | 2382 | 3.281918744 | 9.83E−07 |
| XXXX | AQKF | 4 | 32 | 2.563234509 | 1.28E−06 |
| XXX | EAP | 6 | 134 | 7.151435288 | 1.33E−06 |
| XX | EY | 27 | 3673 | 2.670114109 | 1.36E−06 |
| X | K | 209 | 61838 | 1.334619444 | 1.41E−06 |
| X . . . X | E . . . Y | 16 | 1495 | 3.99483461 | 1.43E−06 |
| XXX | EAL | 7 | 222 | 5.036070796 | 1.82E−06 |
| XXX | EAF | 7 | 225 | 4.968923186 | 1.99E−06 |
| XX | SG | 55 | 11062 | 1.805992828 | 2.99E−06 |
| X . . . X | E . . . E | 20 | 2529 | 2.651169338 | 3.09E−06 |
| X . . . X | E . . . R | 18 | 1993 | 3.371205449 | 3.42E−06 |
| X.X | E.N | 22 | 2800 | 2.934298684 | 4.01E−06 |
| X . . . X | A . . . G | 31 | 5031 | 2.29999764 | 4.16E−06 |
| X.X | A.Q | 22 | 2838 | 2.895009272 | 4.94E−06 |
| XXXXX | DPAQL | 2 | 2 | 1.781310837 | 5.99E−06 |
| XXXXX | KGYNR | 2 | 2 | 1.781310837 | 5.99E−06 |
| XXXXX | RVNHG | 2 | 2 | 1.781310837 | 5.99E−06 |
| XXXXX | VAQKF | 2 | 2 | 1.781310837 | 5.99E−06 |
| XXXXX | VPKRS | 2 | 2 | 1.781310837 | 5.99E−06 |
| X . . . X | E . . . F | 21 | 2737 | 2.86394393 | 6.53E−06 |
| X.X | G.K | 22 | 2910 | 2.823380177 | 7.25E−06 |
| XXX | EAQ | 7 | 286 | 3.909117891 | 9.55E−06 |
| XXX | ESV | 5 | 111 | 7.194386852 | 1.00E−05 |
| X . . . X | E . . . V | 16 | 1796 | 3.165079424 | 1.07E−05 |
| XX | AS | 24 | 3505 | 2.487197115 | 1.60E−05 |
| XXXXX | SNKVF | 2 | 3 | 1.187540558 | 1.79E−05 |
| XXXX | EAAY | 2 | 3 | 13.67058405 | 1.84E−05 |
| XXX | QKF | 8 | 441 | 2.897331304 | 1.94E−05 |
| X . . . X | R . . . K | 16 | 1856 | 3.217822059 | 2.05E−05 |
| XXXX | DPAQ | 3 | 22 | 2.796255828 | 2.26E−05 |
| X . . . X | E . . . G | 29 | 4896 | 2.210938196 | 2.36E−05 |
| X.X | A.P | 19 | 2472 | 2.870415747 | 2.38E−05 |
| X . . . X | E . . . V | 18 | 2315 | 2.902294799 | 2.46E−05 |
| XXX | EVG | 6 | 225 | 4.259077016 | 2.57E−05 |
| X . . . X | E . . . A | 9 | 645 | 4.677772504 | 2.58E−05 |
| XXX | NKV | 8 | 464 | 2.753713588 | 2.78E−05 |
| XX | AA | 18 | 2274 | 2.875206427 | 2.92E−05 |
| XXX | RAQ | 6 | 233 | 4.112842612 | 3.13E−05 |
| XXX | ESY | 5 | 144 | 5.545673198 | 3.52E−05 |
| XXX | EQV | 6 | 239 | 4.009591333 | 3.60E−05 |
| XXXX | EAAV | 2 | 4 | 10.25293803 | 3.68E−05 |
| XXXX | ESAP | 2 | 4 | 10.25293803 | 3.68E−05 |
| XXX | PNK | 7 | 354 | 3.158213889 | 3.72E−05 |
| XX | SA | 22 | 3233 | 2.471746695 | 3.87E−05 |
| XX | RS | 29 | 5019 | 2.09878421 | 5.08E−05 |
| XXXX | GNKH | 3 | 30 | 2.050587607 | 5.88E−05 |
| XXXX | AVQG | 2 | 5 | 8.202350427 | 6.12E−05 |
| XXXX | EALA | 2 | 5 | 8.202350427 | 6.12E−05 |
| XXXX | EAVK | 2 | 5 | 8.202350427 | 6.12E−05 |
| XXXX | ERVN | 2 | 5 | 8.202350427 | 6.12E−05 |
| XXXX | ESRA | 2 | 5 | 8.202350427 | 6.12E−05 |
| XXXX | EVGK | 2 | 5 | 8.202350427 | 6.12E−05 |
| X . . . X | E . . . F | 14 | 1720 | 2.728700628 | 6.79E−05 |
| X . . . X | E . . . H | 14 | 1665 | 2.987340729 | 6.88E−05 |
| X | G | 318 | 110872 | 1.132587685 | 8.12E−05 |
| X . . . X | A . . . P | 16 | 2079 | 2.864941643 | 8.96E−05 |
| XXXX | EASR | 2 | 6 | 6.835292023 | 9.16E−05 |
| XXXX | EKAF | 2 | 6 | 6.835292023 | 9.16E−05 |
| XXXX | EQGR | 2 | 6 | 6.835292023 | 9.16E−05 |
| XXXX | ESAG | 2 | 6 | 6.835292023 | 9.16E−05 |
| XXXX | EVSN | 2 | 6 | 6.835292023 | 9.16E−05 |
| X . . . X | A . . . K | 15 | 1894 | 2.956182884 | 9.31E−05 |
| X.X | E.H | 20 | 2996 | 2.493032017 | 9.94E−05 |
| X . . . X | S . . . S | 20 | 3109 | 2.401205268 | 0.000117428 |
| XXXX | EAHK | 2 | 7 | 5.858821734 | 0.000128036 |
| XXXX | EASA | 2 | 7 | 5.858821734 | 0.000128036 |
| XXXX | EAVV | 2 | 7 | 5.858821734 | 0.000128036 |
| X . . . X | A . . . Q | 12 | 1347 | 3.165079424 | 0.00013183 |
| XXX | GNK | 6 | 304 | 3.152277397 | 0.000134768 |
| X . . . X | S . . . K | 12 | 1355 | 3.146392608 | 0.000139145 |
| XXX | GQK | 6 | 309 | 3.101269672 | 0.00014718 |
| X . . . X | R . . . K | 17 | 2426 | 2.608605536 | 0.000164821 |
| XXXX | EAFQ | 2 | 8 | 5.126469017 | 0.000170433 |
| XXXX | EVGS | 2 | 8 | 5.126469017 | 0.000170433 |
| X . . . X | E . . . L | 15 | 2051 | 2.598343481 | 0.000174221 |
| X . . . X | K . . . G | 29 | 5498 | 1.963557164 | 0.000178205 |
| XX | PN | 24 | 4100 | 2.126250217 | 0.000203741 |
| XXXX | EAHP | 2 | 9 | 4.556861349 | 0.000218766 |
| XXXX | EAVA | 2 | 9 | 4.556861349 | 0.000218766 |
| XXXX | ESYK | 2 | 9 | 4.556861349 | 0.000218766 |
| XXXX | EVPS | 2 | 9 | 4.556861349 | 0.000218766 |
| X . . . X | A . . . N | 11 | 1221 | 3.20072221 | 0.00022569 |
| X . . . X | A . . . G | 30 | 5790 | 1.928825672 | 0.000238912 |
| X . . . X | S . . . G | 28 | 5356 | 1.946111638 | 0.000262382 |
| X . . . X | Q . . . K | 14 | 1865 | 2.802006983 | 0.00026908 |
| XXXX | ALNK | 2 | 10 | 4.101175214 | 0.000273006 |
| XXXX | EAHA | 2 | 10 | 4.101175214 | 0.000273006 |
| XXXX | EARA | 2 | 10 | 4.101175214 | 0.000273006 |
| XXXX | EAVP | 2 | 10 | 4.101175214 | 0.000273006 |
| XXXX | EKGY | 2 | 10 | 4.101175214 | 0.000273006 |
| XXXX | EQHA | 2 | 10 | 4.101175214 | 0.000273006 |
| X . . . X | A . . . F | 18 | 2827 | 2.376658104 | 0.000284653 |
| XX | AK | 24 | 4264 | 2.044471362 | 0.000320214 |
| X.X | S.N | 18 | 2793 | 2.406806849 | 0.000327002 |
| XX | KG | 24 | 4274 | 2.039687854 | 0.000329639 |
| XXXX | EAEP | 2 | 11 | 3.728341103 | 0.000333122 |
| XXXX | ERGA | 2 | 11 | 3.728341103 | 0.000333122 |
| XXXX | ESGY | 2 | 11 | 3.728341103 | 0.000333122 |
| XXXX | ESQV | 2 | 11 | 3.728341103 | 0.000333122 |
| XXXX | EYQA | 2 | 11 | 3.728341103 | 0.000333122 |
| XXXX | GQKQ | 2 | 11 | 3.728341103 | 0.000333122 |
| XXXX | GSFG | 2 | 11 | 3.728341103 | 0.000333122 |
| XXXX | PRAQ | 2 | 11 | 3.728341103 | 0.000333122 |
| X . . . X | E . . . H | 10 | 1111 | 3.017465012 | 0.000335336 |
| XXXX | NKVS | 3 | 56 | 1.098529075 | 0.000382622 |
| XXXX | QKRV | 3 | 56 | 1.098529075 | 0.000382622 |
| XXXX | EAGY | 2 | 12 | 3.417646011 | 0.000399087 |
| XXXX | ERPN | 2 | 12 | 3.417646011 | 0.000399087 |
| XXXX | NVPR | 2 | 12 | 3.417646011 | 0.000399087 |
| XXXX | VKQH | 2 | 12 | 3.417646011 | 0.000399087 |
| X . . . X | A . . . N | 13 | 1719 | 2.822847973 | 0.000410148 |
| XXXX | AHKL | 2 | 13 | 3.154750164 | 0.00047087 |
| XXXX | ASRE | 2 | 13 | 3.154750164 | 0.00047087 |
| XXXX | ESAQ | 2 | 13 | 3.154750164 | 0.00047087 |
| XXXX | NRVK | 2 | 13 | 3.154750164 | 0.00047087 |
| XXXX | QKYF | 2 | 13 | 3.154750164 | 0.00047087 |
| X . . . X | E . . . F | 19 | 3165 | 2.234756316 | 0.000471345 |
| X.X | S.R | 18 | 2912 | 2.308451762 | 0.000527898 |
| XXXX | AQKY | 2 | 14 | 2.929410867 | 0.000548441 |
| XXXX | EAAW | 2 | 14 | 2.929410867 | 0.000548441 |
| XXXX | EVNK | 2 | 14 | 2.929410867 | 0.000548441 |
| XXXX | QKRP | 2 | 14 | 2.929410867 | 0.000548441 |
| XXXX | SRSP | 2 | 14 | 2.929410867 | 0.000548441 |
| X.X | E.K | 18 | 2924 | 2.298977951 | 0.000553081 |
| X.X | K.S | 25 | 4711 | 1.981830801 | 0.000568759 |
| X . . . X | A . . . K | 9 | 980 | 3.078738026 | 0.000570104 |
| XXXX | AGAQ | 2 | 15 | 2.734116809 | 0.000631772 |
| XXXX | VNHG | 2 | 15 | 2.734116809 | 0.000631772 |
| XXXX | RSFG | 2 | 16 | 2.563234509 | 0.000720834 |
| X . . . X | A . . . N | 15 | 2283 | 2.445882751 | 0.000744441 |
| XXXX | NRVP | 2 | 17 | 2.412456008 | 0.000815598 |
| XXXX | RAQD | 2 | 17 | 2.412456008 | 0.000815598 |
| XXXX | RGQK | 2 | 17 | 2.412456008 | 0.000815598 |
| XXXX | RVNH | 2 | 17 | 2.412456008 | 0.000815598 |
| XXXX | AYKL | 2 | 18 | 2.278430674 | 0.000916035 |
| XXXX | HGPN | 2 | 18 | 2.278430674 | 0.000916035 |
| XXXX | KFQK | 2 | 18 | 2.278430674 | 0.000916035 |
| XXXX | EANG | 2 | 19 | 2.15851327 | 0.001022116 |
| XXXX | LGNK | 2 | 19 | 2.15851327 | 0.001022116 |
| XXXX | RYAQ | 2 | 19 | 2.15851327 | 0.001022116 |
| XXXX | EAHG | 2 | 20 | 2.050587607 | 0.001133812 |
| XXXX | EARG | 2 | 20 | 2.050587607 | 0.001133812 |
| XXXX | GPNV | 2 | 20 | 2.050587607 | 0.001133812 |
| XXXX | LSPN | 2 | 20 | 2.050587607 | 0.001133812 |
| XXXX | RPNK | 2 | 20 | 2.050587607 | 0.001133812 |
| XXXX | VPKR | 2 | 21 | 1.952940578 | 0.001251095 |
| XXXX | EAQS | 2 | 22 | 1.864170552 | 0.001373937 |
| XXXX | EQVG | 2 | 22 | 1.864170552 | 0.001373937 |
| XXXX | EYKH | 2 | 22 | 1.864170552 | 0.001373937 |
| XXXX | SYKD | 2 | 22 | 1.864170552 | 0.001373937 |
| XXXX | VAQK | 2 | 22 | 1.864170552 | 0.001373937 |
| XXXX | PAQF | 2 | 23 | 1.783119658 | 0.001502309 |
| XXXX | RAQK | 2 | 23 | 1.783119658 | 0.001502309 |
| XXXX | HAKR | 2 | 24 | 1.708823006 | 0.001636184 |
| XXXX | SNKV | 2 | 24 | 1.708823006 | 0.001636184 |
| XXXX | VANQ | 2 | 25 | 1.640470085 | 0.001775532 |
| XXXX | PYNF | 2 | 26 | 1.577375082 | 0.001920326 |
| XXXX | DNKV | 2 | 27 | 1.518953783 | 0.002070539 |
| XXXX | EANR | 2 | 27 | 1.518953783 | 0.002070539 |
| XXXX | EPLG | 2 | 28 | 1.464705433 | 0.002226142 |
| XXXX | GYNR | 2 | 28 | 1.464705433 | 0.002226142 |
| XXXX | PAQL | 2 | 28 | 1.464705433 | 0.002226142 |
| XXXX | PNKG | 2 | 28 | 1.464705433 | 0.002226142 |
| XXXX | PNQG | 2 | 28 | 1.464705433 | 0.002226142 |
| XXXX | SNRV | 2 | 28 | 1.464705433 | 0.002226142 |
| XXXX | VSSD | 2 | 28 | 1.464705433 | 0.002226142 |
| XXXX | EASG | 2 | 30 | 1.367058405 | 0.002553407 |
| XXXX | SNRH | 2 | 30 | 1.367058405 | 0.002553407 |
| XXXX | PKRS | 2 | 33 | 1.242780368 | 0.003084045 |
| XXXX | AWNK | 2 | 34 | 1.206228004 | 0.003271413 |
| XXXX | VPSE | 2 | 35 | 1.171764347 | 0.003463979 |
| XXXX | ANVS | 2 | 36 | 1.139215337 | 0.003661716 |
| XXXX | PKHS | 2 | 38 | 1.079256635 | 0.0040726 |
| XXXX | AKRS | 2 | 39 | 1.051583388 | 0.004285693 |
| XXXX | YNQK | 2 | 39 | 1.051583388 | 0.004285693 |
| X | Q | 178 | 60270 | 1.166233219 | 0.006243371 |
| X | R | 179 | 63542 | 1.112394281 | 0.030076053 |

In each of Tables 1 and 2:

“n”=the number of times the motif occurs in the top correlating predictive peptides. For Age, A total of 71 peptides were found to be statistically significant and with log 10(old/young) >0.2 in both experiments with 601 discovery set and 1074 validation set of samples, where old was age >65 and young was age <40. For BMI, A total of 331 peptides were found to be statistically significant and with log 10(high/low)>0.1 in both experiments with 601 discovery set and 1074 validation set of samples, where high is BMI>33 and low is BMI <24. Since “n” is total count of motif, not number of peptides with motif, it is possible to have n that is greater than number of peptides. For example, there are 170 serines in 71 peptide probes.

n. lib=the number of times the motif occurs in the array library

“enrich”=the fold enrichment of a motif in the top correlating predictive peptides relative to the number of times the motif occurs in the array library.

p=the statistical significance of the occurrence of a motif in the top correlating predictive peptides.

Fold enrichment=(no of times a motif (e.g. ABCD) occurs in the list/no of times the motif (ABCD) occurs in the library)/(Total no the motif type (e.g. tetramer) occurs in the list/over total number the motif type (e.g. tetramers) occurs in library). Percent enrichment is “enrichment” X 100.

The SS submotif was found to occur at the N-terminus and in about 75% of age-associated probes (183/248 of age-associated probes start with SS; only about 0.35% of probes start with SS). This peptide sequence signal remained strong after computationally trimming the N-terminus (predictive power >0.8 AUC). The E[A/S/R/Y/V] submotif was found to occur at the N-terminus and in about 75% of BMI-associated probes (244/322; only about 2% of probes start with E[A/S/R/Y/V]). This peptide sequence signal remained strong after computationally trimming the N-terminus (predictive power >0.7 AUC).

### Example 5. Probe Design

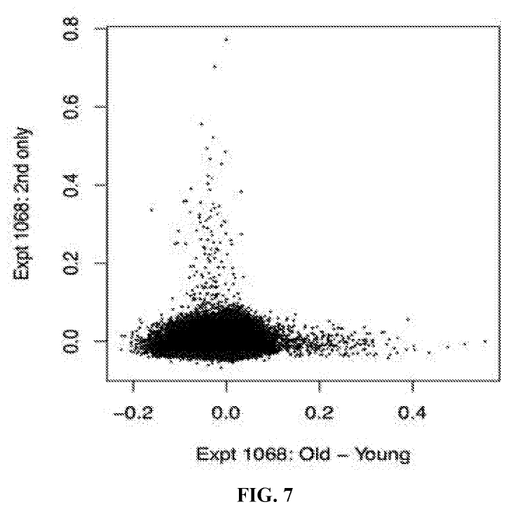

The peptide array probes were evaluated to account for nonspecific or secondary binding due to the possibility that the probes may not represent novel age-associated antigens (e.g. probes may represent elderly serum that allows for more promiscuous binding). FIG. 7 shows the level of binding of secondary antibody in the absence of serum as the level of signal of antibody peptide binding (y-axis) as a function of the difference in years between ‘old’ subjects being older than 60 years and ‘young’ subjects being younger than 40 years given as log ratio. The lack of correlation indicates that secondary antibody binding does not contribute to the identification of the age-associated probes.

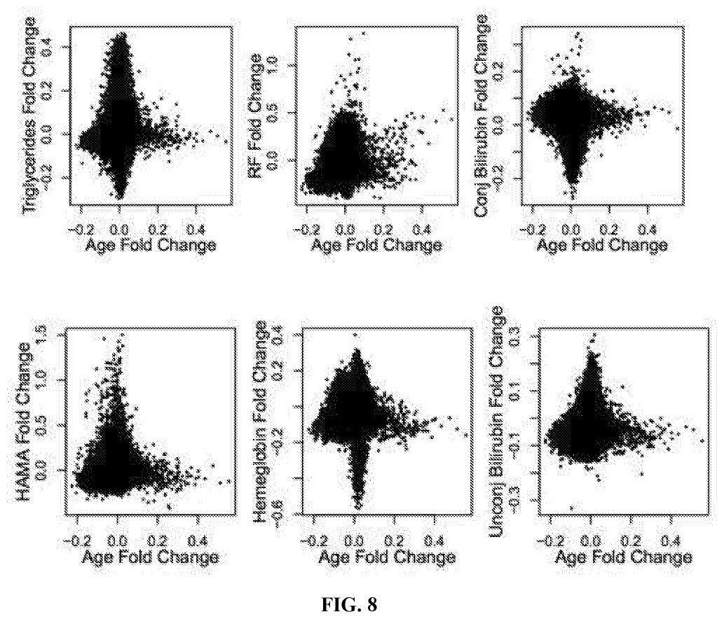

Binding to the age-associated peptides was also assessed for influence from interfering substances. As shown in FIG. 8, the log₁₀ chronological age fold change for the age-associated probes (x-axis) was plotted against the log₁₀ level of triglycerides, rheumatoid factor (RF), conjugated bilirubin, HAMA (human anti-mouse antibodies), hemoglobin, and unconjugated bilirubin (y-axis). A subset of age-associated probes was found to be associated with rheumatoid factor. However, the number of probes overlapping was not statistically significant. Rheumatoid factor is an autoantibody against the Fc portion of IgG, which comprises several SS motifs, including the SSV submotif. However, the rheumatoid factor IgM Fab binds far away from these regions. Therefore, these data indicate that the binding to age-associated peptides is minimally influenced by interfering substances.

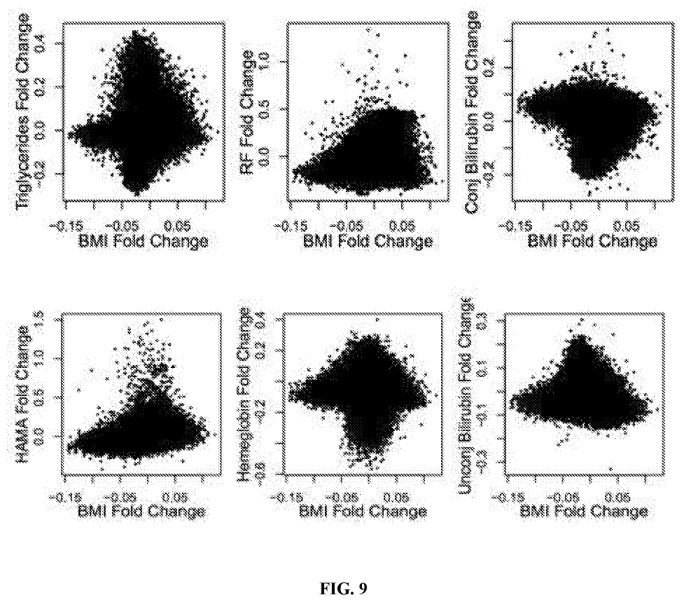

The BMI-associated probes were also evaluated for association with triglycerides, rheumatoid factor, conjugated bilirubin, HAMA, hemoglobin, and unconjugated bilirubin. As shown in FIG. 9, the BMI-associated probes were not influenced by any such potentially interfering substances.

### Example 6. Training Procedure for Predictive Models or Classifiers

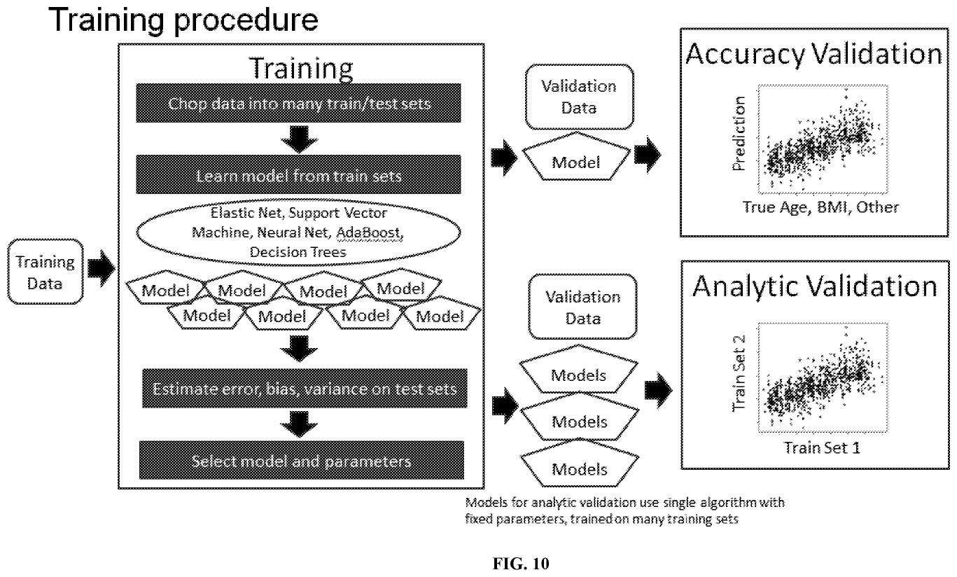

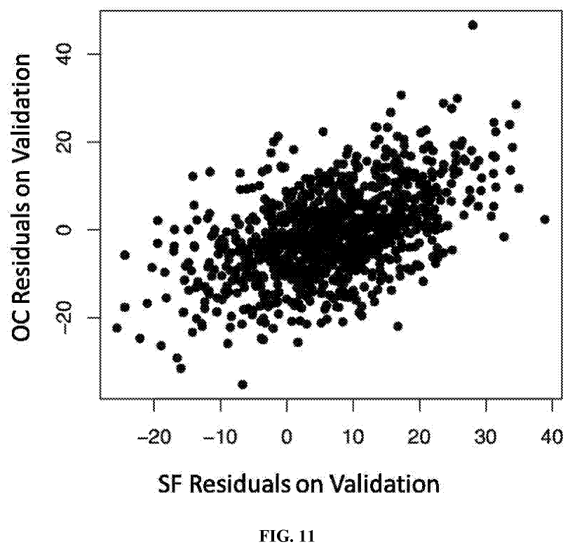

FIG. 10 shows an exemplary process by which models or classifiers are trained and selected.

Data is obtained from one or more sample sets such as through the methods described in Example 1. The data is sorted into multiple training and test sets. The training data sets are used to learn or train one or more models that can be selected from elastic net, support vector machine, neural net, AdaBoost (a machine learning meta-algorithm), or decision trees. The error, bias, and variance are estimated for the various models using the test sets, and these estimates are used to select the model and parameters. The selected model and parameters are then validated for accuracy (e.g., comparing true age, BMI, or other parameter with the prediction). The models can also undergo analytic validation in which the models use single algorithm with fixed parameters, rained on many training sets.

### Example 7. Evaluating the Residuals on the Validation Set

The immune index was evaluated for several conditions that show that residuals can be used as a meaningful indicator of immune health or wellness. The conditions include determining that (1) the regression model is highly accurate; (2) the residuals on the validation set are similar across multiple independent training sets; and (3) the residuals correlate with meaningful phenotypes.

As described in Example 1, and illustrated in FIG. 3, the absolute value and dynamic of the regression output was very similar in different cohorts, which demonstrates the high accuracy of the regression model.

In FIG. 11, the residuals are correlated across multiple training sets to provide a summary of the bias-variance tradeoff. First, a model M_{SF} was trained on San Francisco (SF) samples was trained on the 305 samples obtained at the SF site in the training set of 601 samples. Similarly, a model M_{OC} was trained on Other California (non-San Francisco Calif. samples, OC) was trained on the samples obtained from the 296 OC sites in the same 601 sample training site. Next, M_{SF} and M_{OC} were tested on the validation cohort of 1074 samples. As shown in FIG. 11, the residuals were correlated between the different training sets, satisfying condition 1. The notation “M_{SF}” is shorthand for the model trained on SF data and M_{OC} is the model trained on OC data.

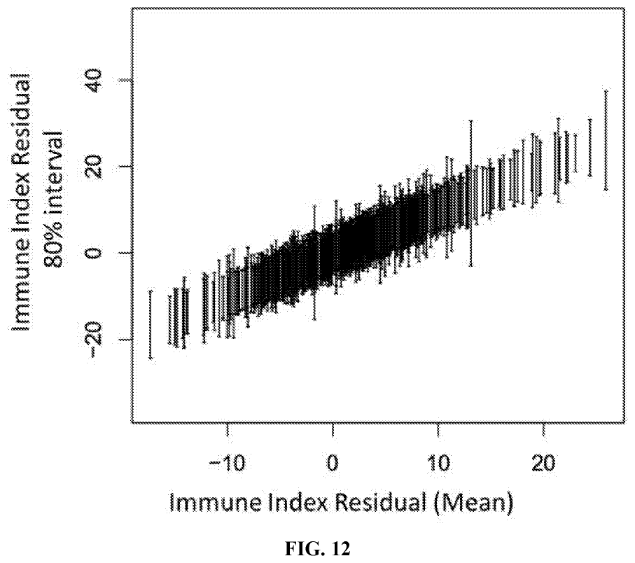

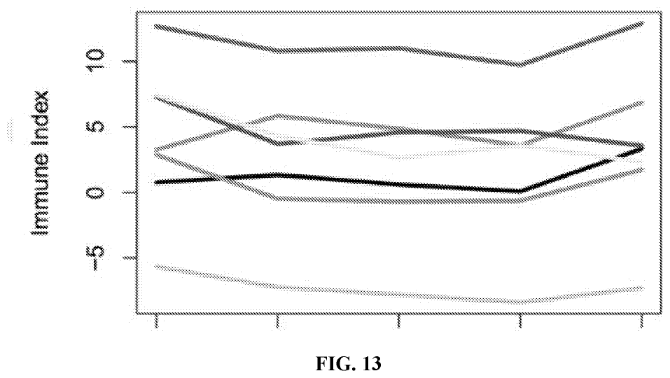

The residual was also shown to be stable for individual samples, which satisfies condition 2. The model was trained with α=0.01 and X=1 using 200 simulated training sets. The residuals were computed on the validation set using 200 trained models. The 80% confidence interval (y-axis) was plotted against the mean of the Immune Index Residual (x-axis), as shown in FIG. 12. It was determined that 97% of samples have an 80% interval that excludes 0 for the immune index residuals >10. The immune index residuals were also found to be longitudinally stable as shown in FIG. 13, which shows the immune index residuals (y-axis) measured over time (x-axis, days 0, 2, 4, 7, 28) for individual donors.

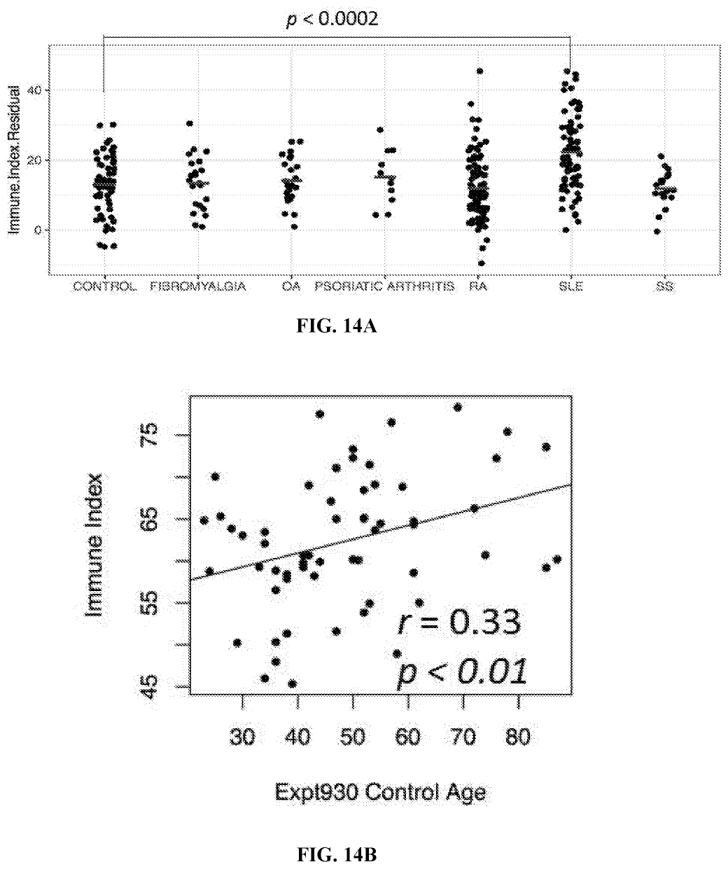

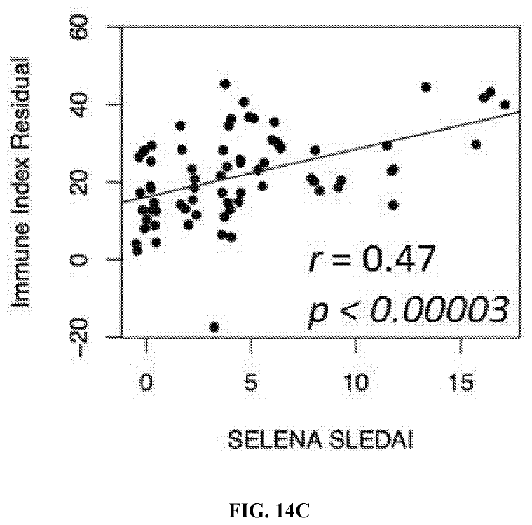

Finally, the residuals were tested for phenotypic association with phenotypes. A prospective study (Expt930) using samples provided from the Albert Einstein College of Medicine was performed using the array binding methods described in Example 1, on a non-acetylated version of the peptide array described in Example 1 (PL9.2 array). Samples were obtained from sets of patients that had previously been diagnosed with Fibromyalgia, Osteoarthritis (OA), Psoriatic arthritis, Rheumatoid arthritis (RA), Lupus (SLE) and Sjogrens (SS). The samples were processed into immune index scores by the prediction algorithm. A linker function was learned specific to the controls of Expt930, since PL9.2 yielded a similar correlation, but distinct absolute values. This linear linker function was then used to calculate immune index residual values compared to expected immune index score for a person's given chronological age (FIG. 14A). These samples were profiled using a PL9.2 array following confirmation of the correlation of Immune Index to chronological age using this array type (FIG. 14B). The correlation of Immune Index values to chronological age was confirmed when binding was evaluated on the PL9.2 array. The immune index was associated with age in the control cohort to provide r=0.33 and p<0.01.

FIG. 14A shows the immune index residual values for each of the disease groups relative to the control group of healthy subjects. A t-test was performed to determine whether residual values could be associated with a disease. The results of the t-test showed that the presence of SLE is associated with higher Immune Index residual i.e. an subject with SLE has an ‘older’ immune system than that of age-matched subjects. Furthermore, the immune index residual was determined to be correlated with the SELENA-SLEDAI score, which is a measure of SLE disease activity (FIG. 14C).

### Example 8. Peptide Microarray Optimization

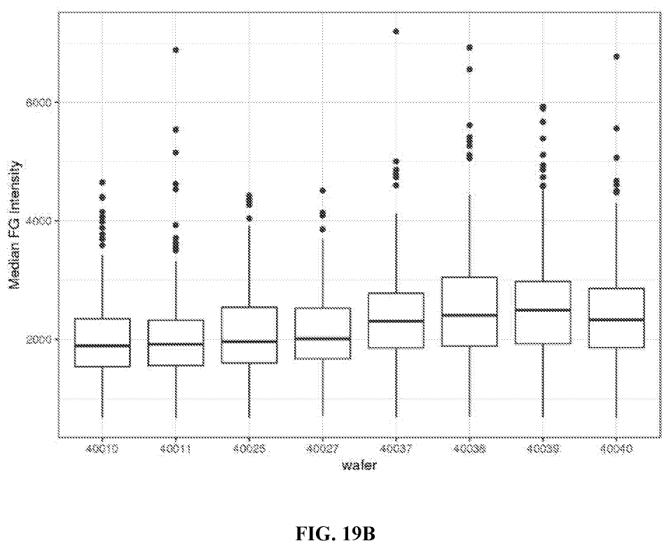

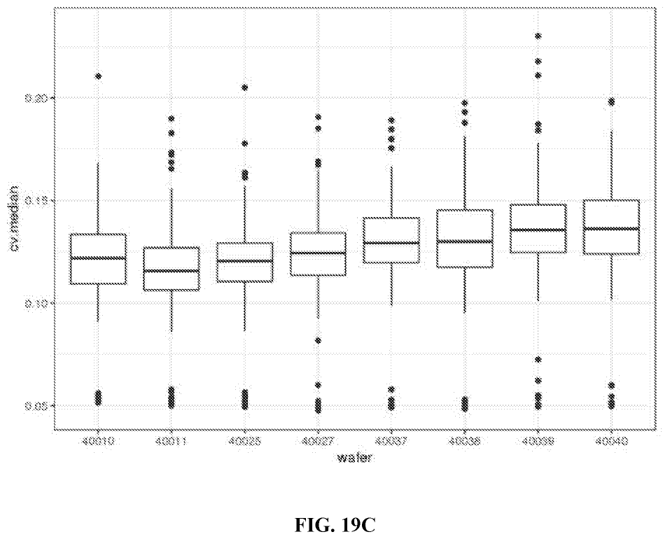

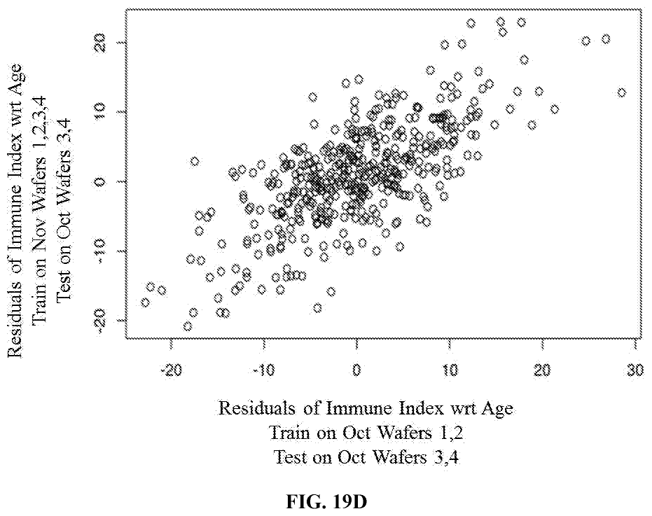

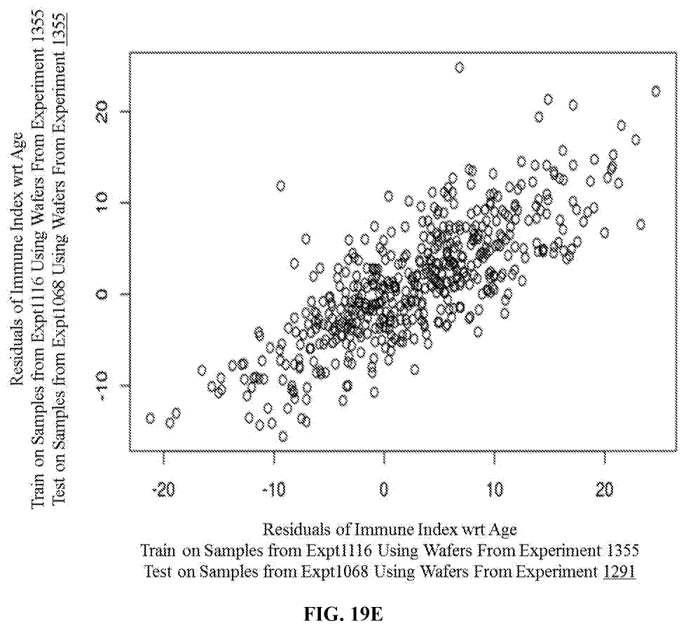

Peptide microarray was synthesized on grids of a surface of a wafer as shown in FIG. 19A. The synthesis and batch of the wafers were continuously optimized by examining for consistent foreground intensity and coefficient of variation (CV) as demonstrated by FIG. 19B and FIG. 19C. Additionally, the same sample sets were tested on different wafers or dates of experiments to validate the correlation of the samples and age as seen in FIG. 19D and FIG. 19E.

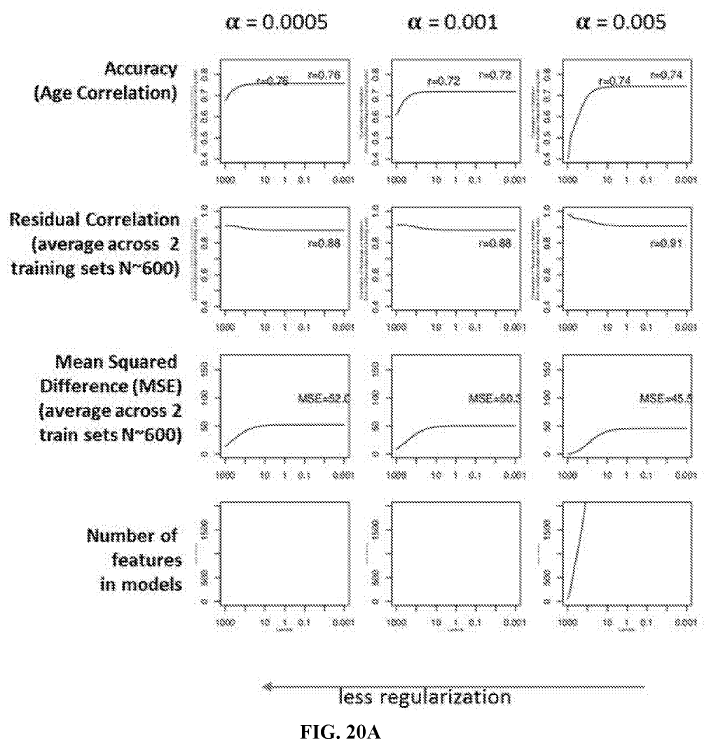

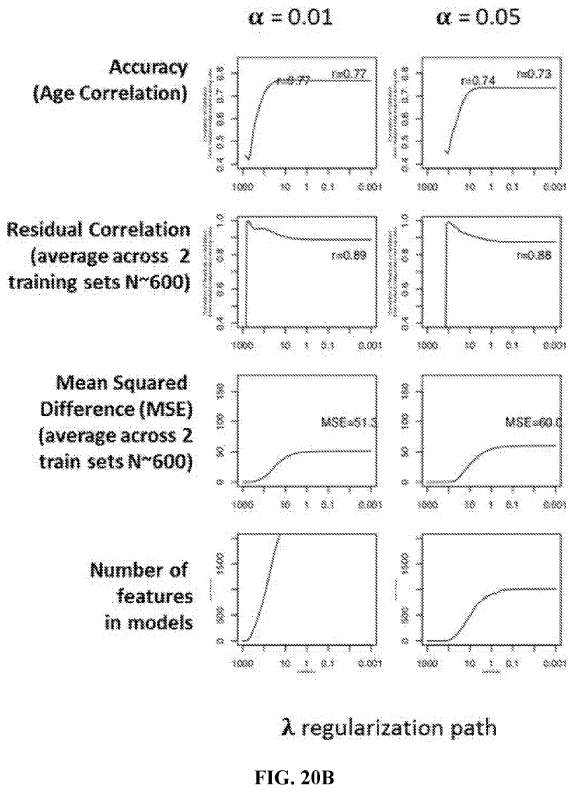

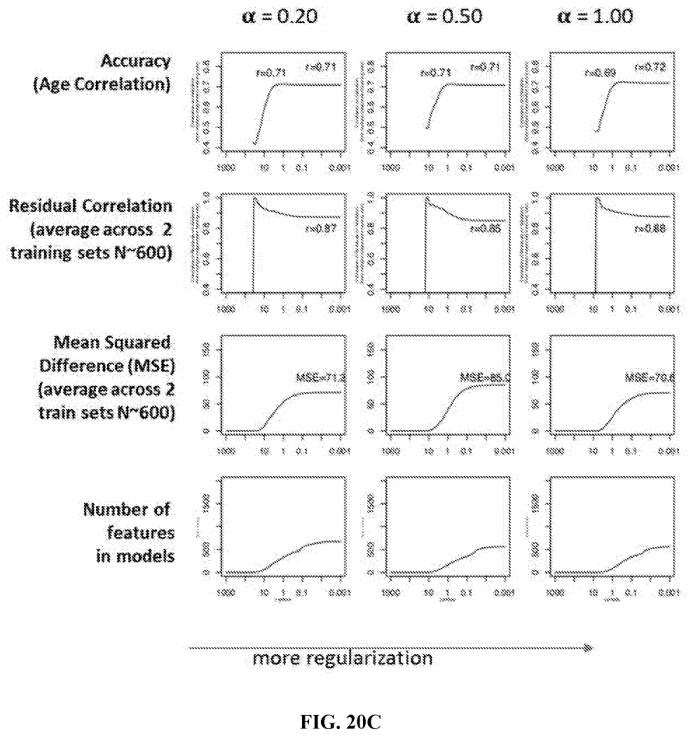

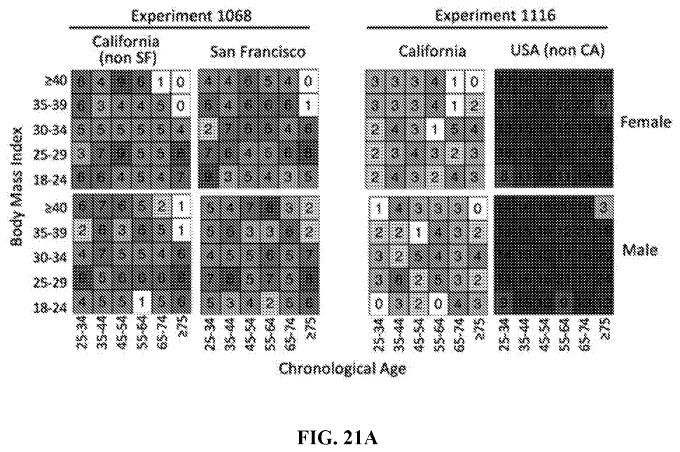

FIG. 20A-20C summarized improvements of data output and analysis based on the peptide microarray conducted in conjunction with the optimizations. The data showed improvements of accuracy, residual correlation, mean squared difference, and number of features in models. FIG. 20A, FIG. 20B, and FIG. 20C show increasing regularization from left to right in order from 20A to 20B and then 20C. The residual correlation is higher across the entire regularization path shown here in FIG. 20 as compared to FIG. 16A.

### Example 9. Aging Modulates Serum Antibody Affinity Profiles

Aging is associated with broad decline in organ function and increased risk for many chronic human diseases. Interestingly, there is high variance in impact of ageing for any given individual. This variance, which is driven by both genetics and environment, has led researchers to search for biomarkers that can predict “biological age”. The use of such biomarkers could potentially enable routine medical screening, prophylactic intervention, and better designed clinical trials. Existing markers, such as telomere length and the epigenetic clock are correlated with age and weakly associated with disease and all-cause mortality (hazard ratios of 1.15-1.3), but neither provide a clinic-ready marker that specifies which physiological or immunological processes are awry.

Ageing of the immune system (e.g., immunosenescence) leads to decreased immunity, loss of immune memory, and ultimately manifests as age-associated latent infection re-activation and decreased efficacy of prophylactic vaccination. Adaptive and innate immune mechanisms are impaired, including decreased cellular proliferation, migration, T-cell receptor diversity, antibody secretion, phagocytic abilities, cytotoxicity, and broad dysregulation of cytokines and chemokines. Loss of CD8+ T cell function results in age-associated decreases in cytotoxic memory and immunity. Generating and maintaining the adaptive humoral response is broadly impacted by ageing. Specifically, plasma cells produce less antibody, germinal center B cell selection results in lower affinity antibodies, CD4+ T cell diversity and T cell hematopoiesis decrease, professional antigen presenting cells reduce expression of peptide-MHC-II complex, and antibody effector cells show decreased functional clearance of antibody-bound pathogens. These dysregulated immune processes are also associated with age-associated increased chronic inflammation, termed “inflammaging”.

Molecular and cellular markers of immunosenescence or immune health have been focused largely on chronic inflammation driven by innate immune mechanisms and T cell surface markers. The most prominent existing inflammation biomarkers have been CRP, IL-6, and IL13, which are all associated with cardiovascular disease and all-cause mortality (hazard ratio=1.7). Inflammation can be compounded by impaired T cell function. Thymic involution, which starts in infancy and typically completes by 50 years of age, results in reduced production of naïve CD4+ and regulatory T cells. These naïve cell populations are maintained for decades in the periphery after thymic emigration, but eventually T cell receptor diversity declines which is hypothesized to lead to lower antibody diversity and decreased humoral protection. The smaller naïve compartment also leads to increased inflammation due to a higher fraction of antigen-experienced and activated T effector cells secreting inflammatory cytokines, even though each individual cell produces less cytokine in response to direct re-stimulation.

There is a significant need for immunosenescence or immune health biomarkers. Immune system decline impacts health. Subclinical immunopathology impacts health, and intervention can mitigate these negative effects. However, metrics for assessing clinical risk of disease or improvement in risks associated with behavioral intervention are limited. There is significant unmet need for such a marker for immunology.

Described herein is an “Immune Age” (or an immune index or an immune index age) biomarker for immunosenescence or immune health. Serum antibody affinities with chronological age from 1675 donors were identified by incubating serum samples on a peptide microarray and subsequently probing with fluorophore-conjugated secondary anti-IgG antibody. A regression model was created, linking peptide fluorescence to chronological age and showed that this score was highly robust with respect to reagents, peptide microarray design, and serum handling. The regression model was tested on an independent cohort of samples to confirm all results. It was show that a donor's predicted ‘immune age’ was longitudinally consistent over years, suggesting that this could be a robust long-term biomarker of humoral immunosenescence or immune health. Additionally, serum from donors was assayed for autoimmune disease and found a significant association between the “immune age” and disease activity.

Results

Serum antibody affinities and cytokine concentration assay development

To develop a biomarker for humoral immunosenescence or immune health, a wide range of analytes that could directly or by proxy provide measurement of plasma cell, memory B cell, and antibody affinity was consider. This included a wide range of cytokine profiling, immunophenotyping by lineage and cell surface markers, immune cell senescence, and methods for measuring antibody binding affinity. To ensure that biomarkers could be deployed at scale, only assays that could be deployed at scale were considered. Therefore, optimized assays for high-throughput cytokine profiling and serum antibody affinity were developed and validated.

To assay antibody affinity, high-density peptide microarrays arrays that captured both proteomic and unbiased diverse antigen discovery were used. The unbiased diverse antigen arrays contained roughly 125,000 distinct peptide sequences and had been found to be effective in predicting disease and chronic infections. In addition to these diverse arrays, a new peptide microarray format was designed to enable synthesis of approximately 3,200,000 peptide sequences, which enabled broadly profiling antibody affinity against millions of known and potential antigens. This dual peptide array strategy enabled detecting known antigens, characterizing binding to the human and infectious proteomes, and discovering uncharacterized novel antigens.

Serum antibody affinities were measured by diluting serum samples, incubating on peptide arrays, probed by fluorophore-conjugated secondary anti-IgG antibody, and subsequent fluorescent intensity detected via imaging. Additionally, assays were developed for cytokine and C-reactive protein (CRP) serum concentration. Serum samples were diluted, incubated with multiplex panels of Luminex beads conjugated to anti-cytokine antibodies, and quantified by dual-laser flow.

Enrollment of a Cohort Experiencing Diverse Stages of Immunosenescence

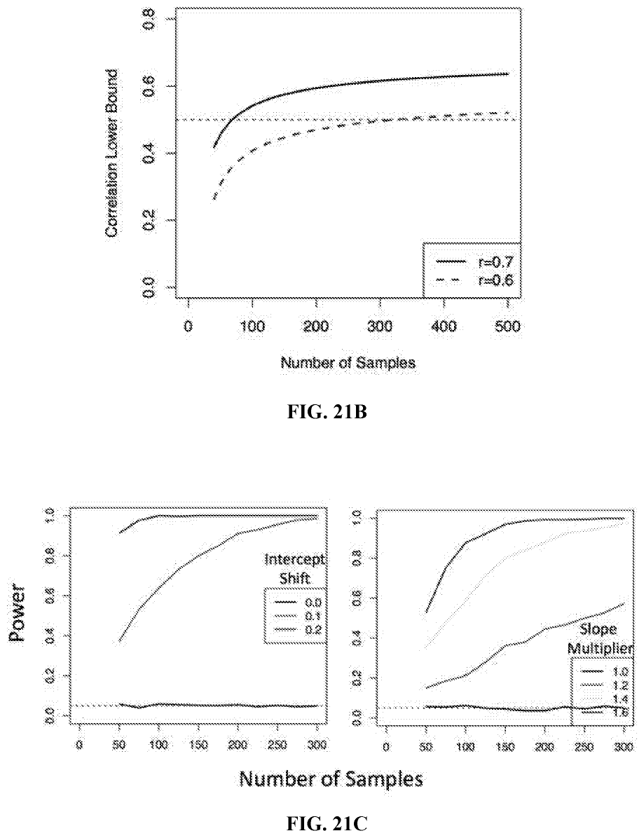

A large cohort of venipuncture serum was collected from donors. FIG. 21A-C outlined the information of the cohort as well as the statistical analysis to identify the necessary size of the cohort and parameters for analyzing the cohort. The donors were annotated for age, weight, height, sex, and collection site. The following annotations were obtained for each donor: age, BMI, sex, ethnicity, location of original venipuncture blood donation, and ethnicity. With the exception of location of blood donation, all other annotations were derived from self-reported age, weight, height, sex, and ethnicity. Age at time of blood donation was calculated by CTS based on self-reported birthdate, actual birthdate was not provided. BMI was calculated as weight (in kilograms) divided by squared height (in meters) in units of kg/m². Sex was self-reported as male or female. Ethnicity was self-reported and then coarsely grouped into White, Latino, Asian, and Black. Site of blood donation was recorded and reported as San Francisco, Other California, Arizona, Nevada, California, North Dakota, Washington, Montana, and South Dakota, Texas.

To minimize bias associated with self-selecting blood donors, donor enrollment was balanced by age, body mass index (BMI), sex, and geography. In total, 1675 samples were obtained for training and verifying a model.

Serum Antibody Affinity and Cytokine Concentrations were Correlated with Chronological Age

Correlations between cytokine concentrations and antibody affinity with age were quantified. Correlations and all downstream statistics were calculated using log-transformed cytokine and peptide array data that incorporated modest amounts of shrinkage to decrease noise in the lower range of values. In each of the analyses, additional analysis was conducted to adjust for BMI and sex.

Cytokines associated with age included Eotaxin, sIL2Ra, or sCD40L. Cytokines correlated with BMI included sIL2RA and CRP. Age and BMI were both weakly correlated with IP10 and sTNFR1 (see Example 2).

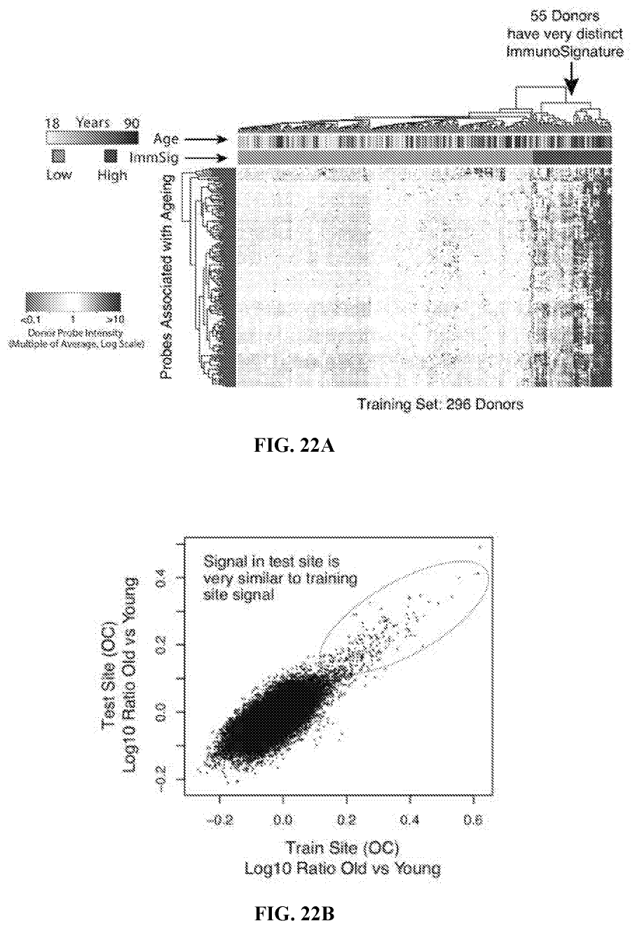

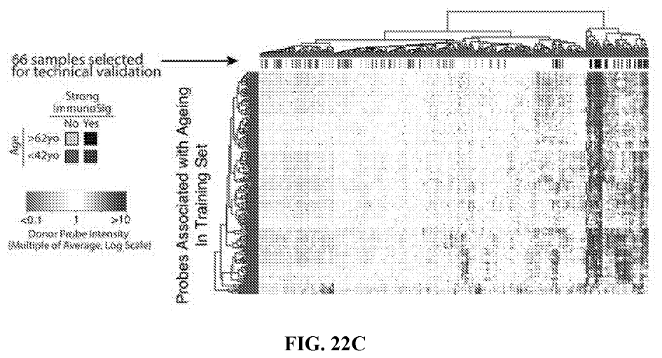

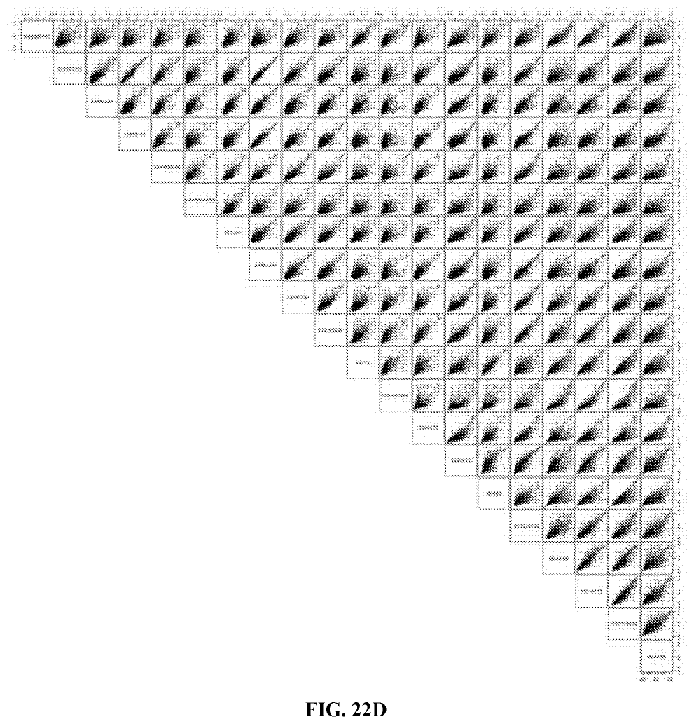

A plurality of peptides was identified to correlate with age and BMI (statistically significant Pearson's correlation). Effect size for age was estimated using (1) log-ratio of average peptide fluorescence >60 yo and <40 yo and (2) the difference of linear model fit at age 25 vs 80. These two measurements were highly correlated as show in FIG. 22. FIG. 22A shows that if a donor had high intensity binding to an SS-feature, it was likely that the donor's serum would also bind highly to other probes that began with “SS”. FIG. 22B shows that the distinct pattern of the imaging mass spectrometry (IMS) was due to more prevalent SS probes in older donors. FIG. 22C shows that high intensity fold change probes were SS probes, which were more abundant in donors who were older than 60 years old than young donors who were younger than 42 years old.

The 300 highest effect size probes (each having log ratio >0.2) were selected for additional testing. Many older individuals had higher and many younger individuals had lower fluorescence at these 300 peptides. Furthermore, the intra-donor correlation of these 300 probes was extremely high, suggesting that the set of antibody clones binding these peptides may have a shared antigen across donors as detailed in FIG. 22.

### Example 10. Age-Associated Antibody Affinity to N-Terminus Serine

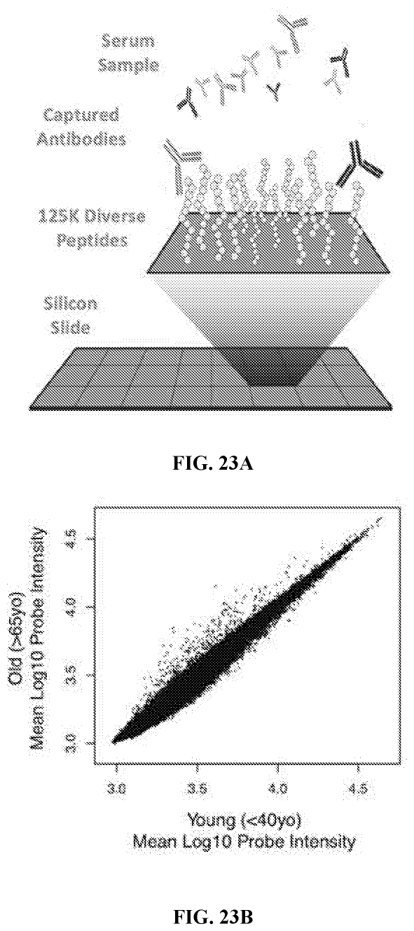

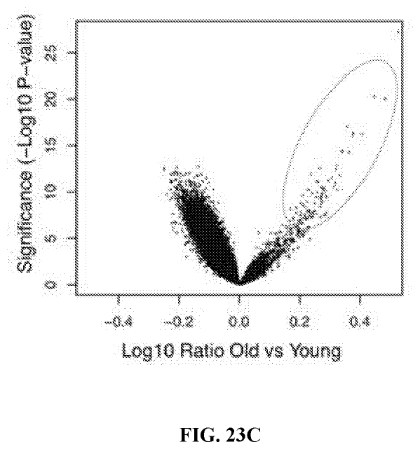

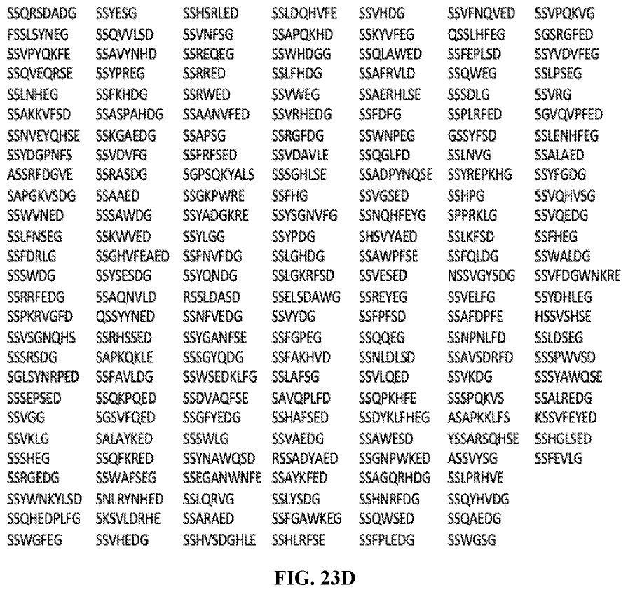

When the 300 largest effect size probes were examined, there were about 280 probes that started with two consecutive serine residues (SS) at the most N-terminus position SS as seen in FIG. 23. The remaining 20 peptides had one serine residue “S” in one of the 2 most N-terminus residues. The diverse peptide microarray had a total of about 2000 probes that started with SS. Of the 1700 probes which were not the largest effect size, there was a highly significant shift in young-old peptide fluorescence log-ratio. These was a much smaller shift for other combinations of N-terminus di-residues, and those with the largest of the small shifts included serine (FIG. 23C). An exemplary list of SS peptides identified in the TIMS for predicting immune age. The list comprises highest ranking SS-peptides, which have the largest fold changes in older, as opposed to younger, donors.

TABLE 3
the exemplary list of SS peptides from FIG. 23C

| | | | | |
|---|---|---|---|---|
| SSQRSDADG | SSYSESDG | SSHVSDGHLE | SSAWPFSE | SSVAEDG |
| FSSLSYNEG | SSAQNVLD | SSLDQHVFE | SSVESED | SSWGSG |
| SSVPYQKFE | QSSYYNED | SSAPQKHD | SSREYEG | SSVPQKVG |
| SSQVEQRSE | SSRHSSED | SSWHDGG | SSFPFSD | SGSRGFED |
| SSLNHEG | SAPKQKLE | SSLFHDG | SSQQEG | SSYVDVFEG |
| SSAKKVFSD | SSFAVLDG | SSVWEG | SSNLDLSD | SSLPSEG |
| SSNVEYQHSE | SSQKPQED | SSVRHEDG | SSVLQED | SSVRG |
| SSYDGPNFS | SGSVFQED | SSRGFDG | SSQPKHFE | SGVQVPFED |
| ASSRFDGVE | SALAYKED | SSVDAVLE | SSDYKLFHEG | SSLENHFEG |
| SAPGKVSDG | SSQFKRED | SSSGHLSE | SSAWESD | SSALAED |
| SSWVNED | SSWAFSEG | SSFHG | SSGNPWKED | SSYFGDG |
| SSLFNSEG | SNLRYNHED | SSYSGNVFG | SSAGQRHDG | SSVQHVSG |
| SSFDRLG | SKSVLDRHE | SSYPDG | SSHNRFDG | SSVQEDG |
| SSSWDG | SSVHEDG | SSLGHDG | SSQWSED | SSFHEG |
| SSRRFEDG | SSHSRLED | SSLGKRFSD | SSFPLEDG | SSWALDG |
| SSPKRVGFD | SSVNFSG | SSELSDAWG | SSVFNQVED | SSVFDGWNKRE |
| SSVSGNQHS | SSREQEG | SSVYDG | QSSLHFEG | SSYDHLEG |
| SSSRSDG | SSRRED | SSFGPEG | SSFEPLSD | HSSVSHSE |
| SGLSYNRPED | SSRWED | SSFAKHVD | SSVWEG | SSLDSEG |
| SSSEPSED | SSAANVFED | SSLAFSG | SSSDLG | SSSPWVSD |
| SSVGG | SSAPSG | SAVQPLFD | SSPLRFED | SSSYAWQSE |
| SSVKLG | SSFRFSED | SSHAFSED | GSSYFSD | SSALREDG |
| SSSHEG | SGPSQKYALS | SSVAEDG | SSLNVG | KSSVFEYED |
| SSRGEDG | SSGKPWRE | RSSADYAED | SSYREPKHG | SSHGLSED |
| SSWNKYLSD | SSYADGKRE | SSAYKFED | SSHPG | SSFEVLG |
| SSQHEDPLFG | SSYLGG | SSLYSDG | SPPRKLG | |
| SSWGFEG | SSFNVFDG | SSFGAWKEG | SSLKFSD | |
| SSYESG | SSYQNDG | SSHLRFSE | SSFQLDG | |
| SSQVVLSD | RSSLDASD | SSVHDG | NSSVGYSDG | |
| SSAVYNHD | SSNFVEDG | SSKYVFEG | SSVELFG | |
| SSYPREG | SSYGANFSE | SSQLAWED | SSAFDPFE | |
| SSFKHDG | SSSGYQDG | SSAFRVLD | SSNPNLFD | |
| SSASPAHDG | SSWSEDKLFG | SSAERHLSE | SSAVSDRFD | |
| SSKGAEDG | SSDVAQFSE | SSFDFG | SSVKDG | |
| SSVDVFG | SSGFYEDG | SSWNPEG | SSSPQKVS | |
| SSRASDG | SSSWLG | SSQGLFD | ASAPKKLFS | |
| SSAAED | SSYNAWQSD | SSADPYNQSE | YSSARSQHSE | |
| SSSAWDG | SSEGANWNFE | SSVGSED | ASSVYSG | |
| SSKWVED | SSLQRVG | SSNQHFEYG | SSLPRHVE | |
| SSGHVFEAED | SSARAED | SHSVYAED | SSQYHVDG | |

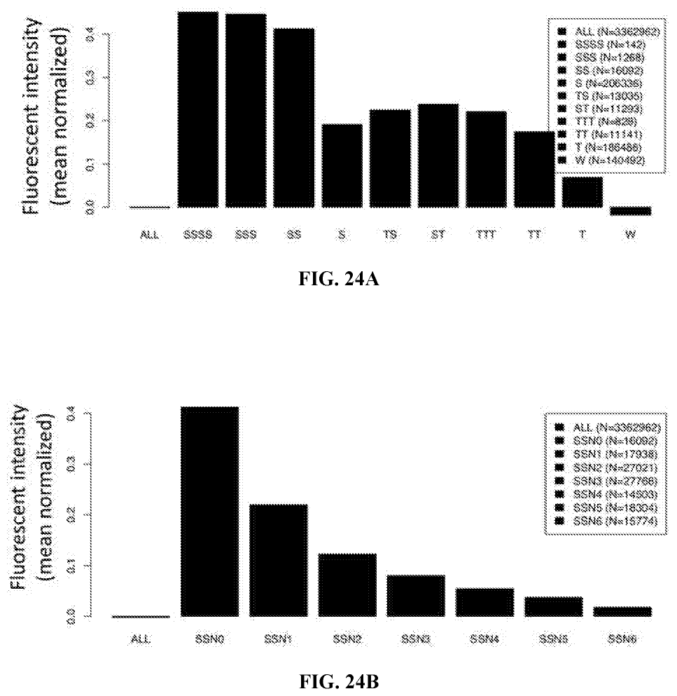

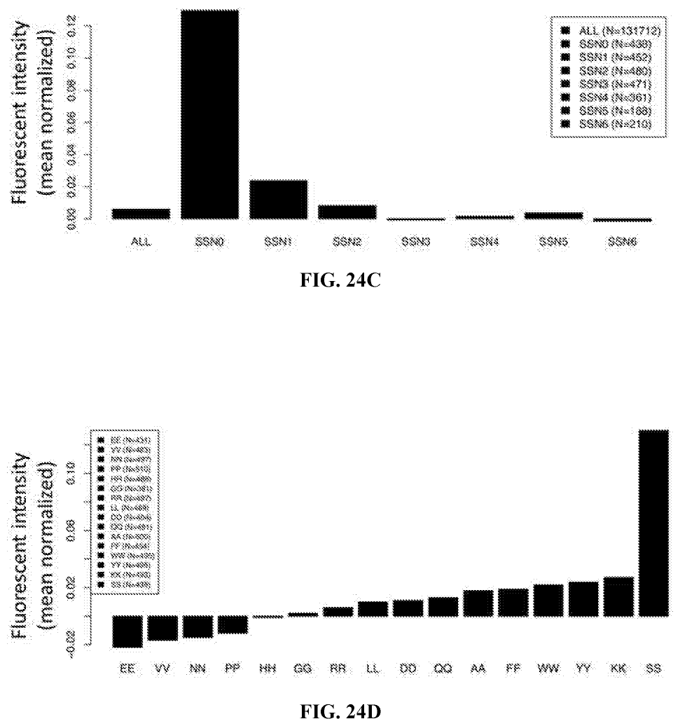

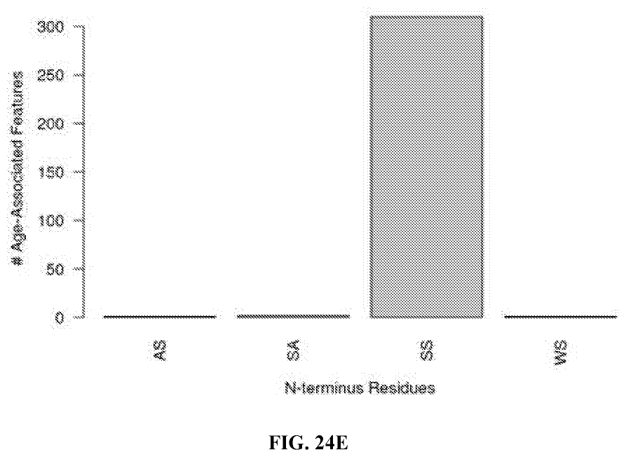

To better refine and understand the age-associated binding to serine, a much broader peptide microarray was developed and assayed with an age-balanced subset of donor serum samples on the 3.2M probes on the new microarray format (V16). It was once again found that “SS” was strongly associated with age. This signal was quantitatively similar to what was found on the diverse array, which was determined by examining ‘SS’ probes originally identified to be associated with age on the diverse array (V13) that were also on the larger array (V16, FIG. 24A). The average differential fluorescence on the two array formats had Pearson's correlation coefficient of r=0.7, which was remarkably high given the differences in manufacturing, SOP, and competing peptide probes. Motif with additional statistically significant enrichments included tetra-serine “SSSS”. FIG. 24A shows that peptides having serine, serine/threonine, or threonine motifs demonstrated higher fluorescence signal than other peptides lacking such motifs. FIG. 24B shows that such motifs positioned on the N-terminus had the strongest signal, while increasing distance from the N-terminus up to 6 residues out still had a stronger signal than peptides lacking such motifs (see “ALL”) (V16). FIG. 24C shows that the peptides having a di-serine motif had higher fluorescence signal compared to other peptides up to 2 residues from the N-terminus in the smaller array (V13 with ˜128 k peptide probes). FIG. 24D shows that peptide probes having a di-serine motif on the N-terminus had significantly larger fluorescence signal compared to other probes. FIG. 24E shows that the vast majority of age-associated peptide probes have an SS N-terminal motif.

FIG. 25 A-D demonstrated that this pattern of “SS” motifs was only present on V13 microarrays where the N-terminus was acetyl-capped (FIG. 25A). This was in contrast to V13 arrays where N-terminus was left as a free-amine. FIG. 25B details the fold change in antibody (Ab) binding signal associated with age obtained in young (<40) v (old (>60) donors when binding was performed on arrays of acetylated or non-acetylated peptides. FIG. 25C illustrates that the Ab binding to two types of arrays each comprising two replicate libraries of peptides. In the first array type, both replicate libraries contained features that were acetylated (Ac/Ac); in the second type of array, only library A was acetylated, while library B was non-acetylated (Ac/NH2). The samples used were derived from young (<40) and old (>60) donors. FIG. 25D shows the acetylation/non-acetylation status of the immuno-signature peptides from which the immune index was determined. The data show that the immune index that was determined for two different populations of donors (sf or oc).

### Example 11. N-Terminus Di-Serine Age-Association Score and Heterogeneity of N-Terminus Di-Serine Age Association

The di-serine N-terminus motif was the most prominent peptide sequence signal. Therefore, the top 291 SS probes associated with age (using the fold change statistic of older vs younger serum donors) were used to calculate the average normalized value of age-associated probes with SS N-terminus (also called the ‘SS score’). This aggregate statistic was robust across experimental assay conditions and peptide microarray format.

The serine age-associated motif was highly heterogeneous, with many older donors not presenting significant antibody binding to probes with di-serine N-terminus. Additionally, a subset of young donors presented with high binding to peptides with di-serine N-terminus. Independent peptide features with di-serine, but different sequence, were highly correlated within donors. In other words, if a donor had 1 SS probe that was high, it was far more likely that other probes ending in SS would also be high (FIG. 22A-C). This pattern was reproduced in technical replicates and on additional microarray formats (V16 and V18). The V18 microarray had 351,909 probes including 138,378 probes that have matching acetylated and un-acetylated peptide features on the array.

### Example 12. Machine Learning Identified Serum Antibody Affinity as Highly Predictive of Chronological Age

Affinity and cytokine data were normalized by mean-centering after taking the log transform. To avoid explicit feature filtering, machine learning regression methods that encouraged sparsity to limit model complexity were used. An exemplary method chosen was chronological age regression with a model that was a weighted linear combination of normalized fluorescent intensities. The machine learning regression model was trained and verified with cross validation and an independent hold out set. The total dataset of ˜1675 donors was split into 675 hold-out samples and 1000 training samples. Of the 1000 training samples, randomly selected 800 were used to train a model and the remaining 200 were used to maximize accuracy on test set. This was repeated 16 times and the average model was subsequently used. Accuracy was then tested on the held out 675 donors. This process was performed 100× to ensure a robust estimate for expected hold-out accuracy. The machine learning regression model was able to accurately predict chronological age, with average Pearson's correlation coefficient r is 0.76.

The model was next optimized for criteria other than accuracy of chronological age prediction. Namely, it was discovered that the Pearson's correlation coefficient r was fairly stable at ˜0.75 independent of selection of model hyperparameters. However, if the method trained on independent training sets and applied both models to an independent hold out test set, the model residuals could vary significantly depending on model hyperparameters.

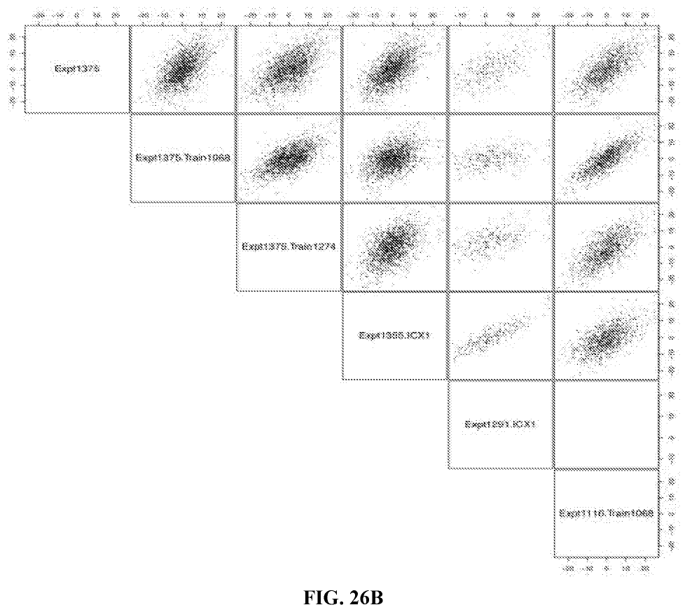

To increase reproducibility, hyperparameter search was performed on reproducibility metrics and found that higher regularization (λ>1) and increased weighting towards L2 norm vs L1 norm. This tilted error toward models that were “underfitted” and “denser”, which resulted in models with lower variance and increased reproducibility. The final model, after optimizing, achieved r approximately at 0.77 and was highly reproducible across a number of array formats, batches, and other considerations. FIG. 26A-B illustrated that the residuals from the machine learning regression model were used to act as proxies for accelerated and decelerated immune aging. A single test set is shown in FIG. 26A, and multiple array formats, syntheses, were used to train the Immune Index (x- and y-axes as labeled). Values on x- and y-axes are residuals, which normalize out the default transitive correlation of all models being correlated to chronological age. FIG. 26B depicts the donor samples examined across 6 different combinations of dates of experimentation, batches of wafer, and array types. The residuals remained correlated across all 6 combinations.

### Example 13. Validation and Reproducibility of Age-Associated Antibody Affinity

The multi-serine affinity and machine learning model for chronological age prediction were both validated using arrays from independently manufactured wafer batches and reagents. The peptide microarray assay was performed by-hand and by the automated integrated system. Samples were processed in a variety of manner and comparable results were found.

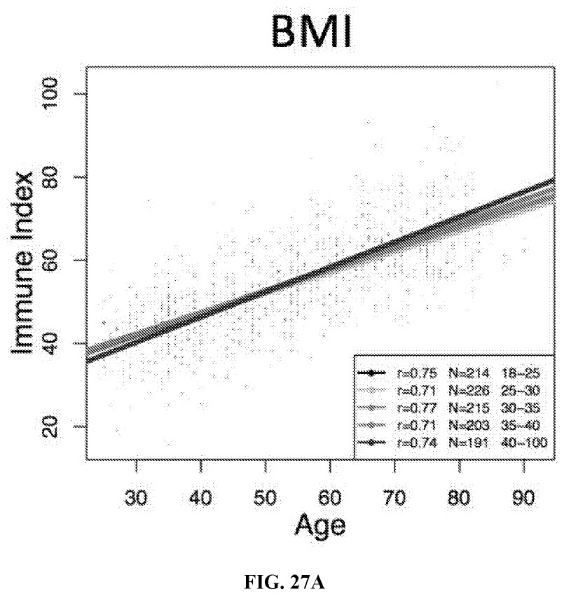

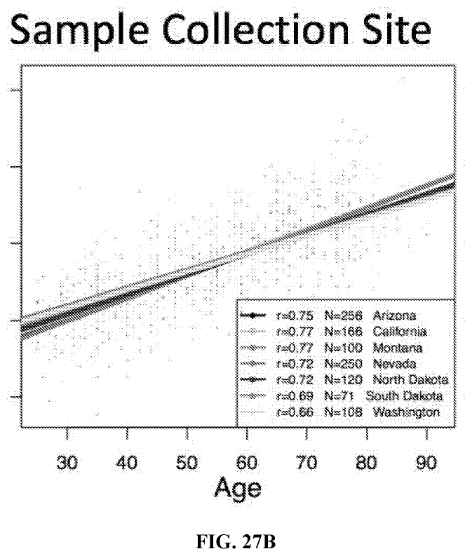

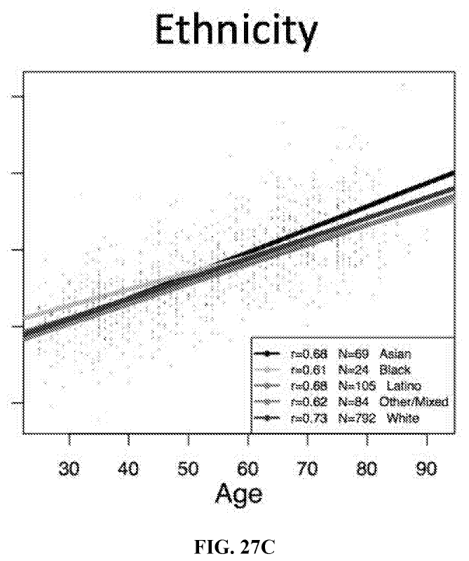

Regression model analysis yielded no statistical significant contrition or alteration imposed by BMI, sample collection site, or ethnicity (FIG. 27). In addition, given the prominent association of chronic inflammation biomarkers with Age and BMI, it was possible that these markers could improve the antibody affinity-based regression model. When cytokines as independent variables were combined with all antibody-affinity peptide features, it was discovered that cytokines did not add predictive capacity. Furthermore, a combination of antibody affinity score and cytokine markers (which dramatically reduced dimensionality) with ridge regression or elastic net still yielded no improvement of prediction on an independent test set. Therefore, cytokine concentrations did not improve prediction of chronological age.

### Example 14. Age-Associated Antibody-Peptide Binding was not Impacted by Endogenous Small Molecules or Proteins

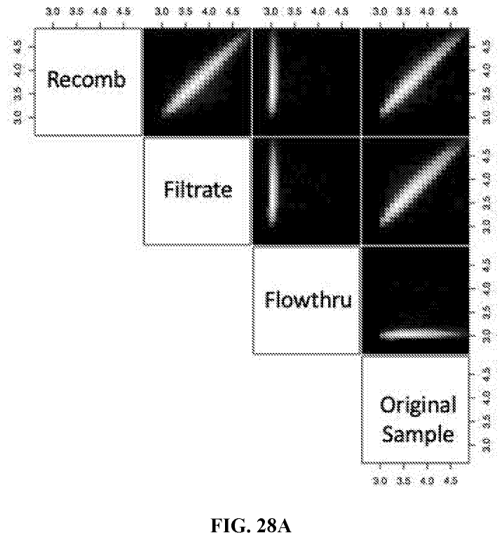

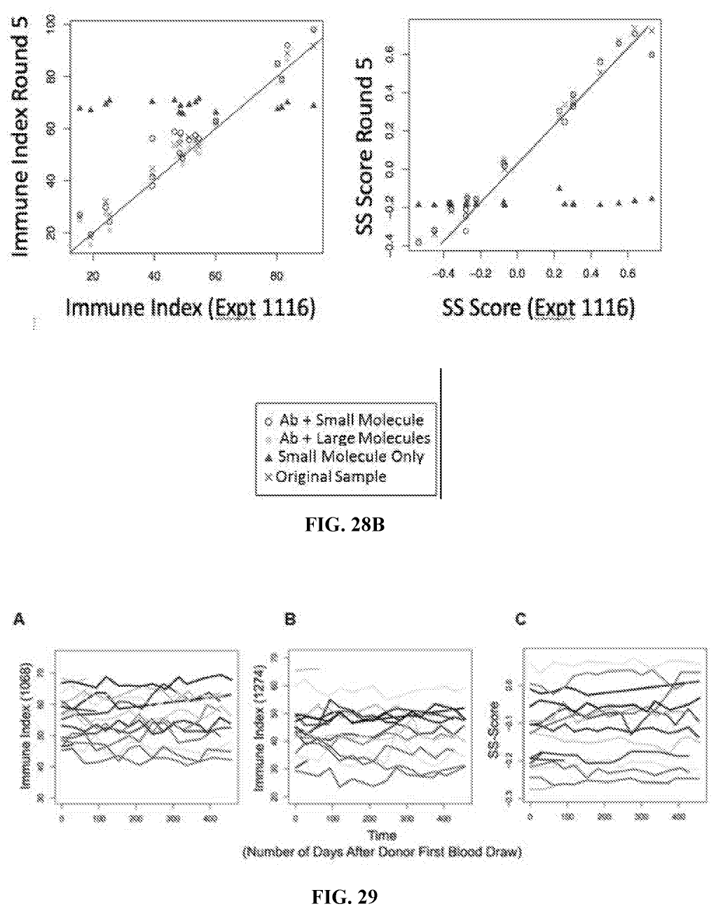

The affinity of IgG molecules binding to arrayed peptides could potentially be impacted by non-immune molecules that were also correlated with age (FIG. 28A-28B). While IgG is a highly abundant serum-protein, there are many small molecules that are present in much greater concentration. It was considered that a non-antibody serum factor may be altering secondary antibody affinity and/or stickiness. Therefore, tests were conducted with depleted antibody (using 30 KDa column filters) and found that the <30 KDa fraction had no signal. Probes that were typically bound by secondary antibody were examined and found that they were not differentially bound in older vs younger donor serum samples.

### Example 15. Age-Associated Antibody Affinity was Stable In Vivo for More than 15 Months

The immune system responds to a number of environmental stimuli, including infectious pathogens, commensal microbial communities, wound healing, and many others. Immunosenescence, when left undeterred, progresses steadily at the population level. Such phenomena should be observed in repeated sampling from a given donor, which did not result in dramatically varying assay and regression results over short time frames. Finding a consistent value for ‘immune age’ would ensure that the value of “immune age” weren't merely detecting an enrichment of transient phenomena in older donors, but rather examining a relatively stable biomarker. A relatively stable biomarker in observational studies is ideal for monitoring in interventional studies. Baseline can be used to predict responder/non-responder. Changes can be interpreted as impactful with fewer donors, since statistical power is higher with the lower biological day-to-day variance.

Blood was drawn from a cohort of donors on an approximately bi-monthly basis and assayed antibody affinity via peptide microarray. It found that nearly all time points for all donors remained in a/−5 year range of their average. The dynamic range of the assay was ˜20-90, so +/−5 yr is <10% of total dynamic range. Similar stability was observed for the SS-score, which was also <10% variation of dynamic range (FIG. 29).

### Example 16. Autoimmune Phenotypes were Associated with Accelerated Humoral Immune Ageing

A cohort of donors (with corresponding health controls) with diverse immune pathologies and phenotypically similar diseases were enrolled for the study. Their pathologies included fibromyalgia, osteoarthritis, psoriatic arthritis, rheumatoid arthritis, systemic lupus erythematosus (SLE), and Sjögren's syndrome. The donor serum was assayed by peptide microarray for calculating the “immune age” metric and normalized by chronological age to obtain an estimate for “accelerated immune aging”.

It was found that SLE donors had accelerated immune ageing, whereas donors from other immune and non-immune diseases were comparable to control donors. In addition, accelerated humoral immunosenescence (e.g., worsening immune health) was found in serum donors with high Lupus disease activity; it was these high disease activity donors that drive the original association between Lupus vs Controls (FIG. 14A-C).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of measuring immune health in a subject comprising, obtaining an immunological measurement from a biological sample from the subject, wherein the immunological measurement comprises antibody-peptide binding.

2. The method of claim 1, wherein the immunological measurement further comprises one or more of the group consisting of: an immune protein sequence, a cytokine level, a metabolite level, and a blood cell count.

3. The method of claim 2, wherein the cytokine is selected from TNFα, GM-CSF, MCP-1 (CCL2), MCP-3, IFNα, IFNγ, IL13, IL2, IL4, IL5, IL6, IL7, IL8, IL10, IL12, IL13, IL17, IL18, IL21, CRP, EGFR, IP10 (CXCL10), Eotaxin (CCL11), MIG, AGP, sTNF-RI, sTNF-RII, sIL2RA, sIL1RA, sIL1RII, sIL6R, CD40L, IL18BP, EGF, VEGF, resistin, leptin, adiponectin, alpha-1-antitrypsin, and free fatty acids.

4. The method of any one of claims 2 to 3, wherein the cytokine is selected from CD40L, EGF, Eotaxin (CCL11), GM-CSF, IFNα, IFNγ, IL-1β, sIL-1RA, sIL-2R, IL-6, IP-10 (CXCL10), MCP-1 (CCL2), TNFα, sTNF-RI, and sTNF-RII.

5. The method of any one of claims 2 to 4, wherein the cytokine is selected from Eotaxin (CCL11), sIL-1RA, sIL-2R, sTNF-RI, IP10 (CXCL10), TNFα, IFNα, IFNγ, IL6, sTNF-RII, and IL-1β.

6. The method of any one of claim 2 to 5, wherein cytokine level is measured in a biological fluid.

7. The method of claim 6, wherein the biological fluid is selected from the group consisting of serum, whole blood, dried blood, plasma, saliva, and a combination thereof.

8. The method of claim 6, wherein the cytokine level is measured in a cytokine assay selected from the group consisting of a bead assay, an aptamer assay, an ELISA assay, and an ELISPOT assay.

9. The method of claim 1, wherein antibody-peptide binding is measured in a peptide array binding assay, wherein the peptide array binding assay comprises
(a) contacting a sample from the subject to a peptide array comprising a plurality of different peptides on distinct features of the array;
(b) detecting the binding of antibodies present in the sample to a set of peptides on the peptide array to obtain a pattern of binding signals, wherein the pattern comprises binding signals each associated with a distinct peptide on the array; and
(c) comparing the pattern of binding signals in the sample to the pattern of binding signals obtained in reference samples, wherein the binding signals obtained from the binding of the sample correspond to a same set of peptides predictive of immune health identified in a plurality of healthy reference subjects,
thereby determining the immune health of the subject.

10. The method of claim 9, wherein the sample is a biological fluid.

11. The method of claim 10, wherein the biological fluid is selected from the group consisting of whole blood, serum, plasma, saliva, and a combination thereof.

12. The method of claim 11, wherein blood is dried blood.

13. The method of any one of claims 9 to 12, wherein the peptide array is a peptide microarray.

14. The method of any one of claims 9 to 13, wherein the peptide array comprises at least about 10,000 distinct peptides.

15. The method of any one of claims 9 to 13, wherein the peptide array comprises at least about 3,000,000 distinct peptides.

16. The method of any one of claims 9 to 15, wherein the peptide array comprises peptides having 20 or fewer amino acids.

17. The method of any one of claims 9 to 15, wherein the peptide array comprises peptides having at least 20 amino acids.

18. The method of any one of claims 9 to 17, wherein the peptide array comprises peptides comprising natural amino acids.

19. The method of any one of claims 9 to 18, wherein the peptide array comprises peptides comprising unnatural amino acids.

20. The method of any one of claims 9 to 19, wherein the peptide array comprises a plurality of peptides characterized by at least one serine motif, threonine motif, serine-threonine motif, or any combination thereof.

21. The method of claim 20, wherein the serine motif comprises S, SS, SSS, or SSSS.

22. The method of claim 20, wherein the threonine motif comprises T or TT.

23. The method of claim 20, wherein the serine-threonine motif comprises TS or ST.

24. The method of any one of claims 20-23, wherein each of the at least one serine motif, threonine motif, or serine-threonine motif is positioned no more than 1, 2, 3, 4, 5 or 6 amino acids from the N-terminus.

25. The method of any one of claims 20-24, wherein the plurality of peptides are acetylated.

26. The method of any one of claims 20-25, wherein a machine learning algorithm generates a prediction of immune health based on the immunological measurement.

27. The method of claim 26, wherein the machine learning algorithm comprises a panel of peptide features comprising the plurality of peptides characterized by at least one serine motif, threonine motif, serine-threonine motif, or any combination thereof.

28. The method of claim 26 or 27, wherein the plurality of peptides make up at least 10%, 20%, 30%, 40%, 50%, 60%, 70%, 80%, 90%, or 95% of the panel of peptide features.

29. The method of any one of claims 26-28, wherein the plurality of peptides comprises at least 50, 100, 150, 200, 250, 300, or 350 peptides.

30. The method of any one of claims 26-29, wherein at least 10%, 20%, 30%, 40%, 50%, 60%, 70%, 80%, 90%, or 95% of the plurality of peptides are statistically correlated with age.

31. The method of claim 30, wherein at least 10%, 20%, 30%, 40%, 50%, 60%, 70%, 80%, 90%, or 95% the plurality of peptides that are statistically correlated with age have an N-terminal di-serine (SS) motif.

32. The method of any one of claims 9 to 19, wherein the peptide array comprises peptides having a sequence comprising EX₁(X₂)ₙ (SEQ ID NO: 1), wherein X₁ comprises an amino acid selected from A, S, R, Y, and V, X₂ comprises any amino acid.

33. The method of claim 32, wherein n is between 3 and 30.

34. The method of claim 32 or claim 33, wherein antibody-peptide binding to a peptide having a sequence of SEQ ID NO: 1 is associated with body mass index (BMI).

35. The method of any one of claims 9 to 34, wherein the peptide array comprises peptides having a sequence comprising SS(X)ₙ (SEQ ID NO: 2), wherein X comprises any amino acid.

36. The method of claim 35, wherein n is between 3 and 30.

37. The method of claim 35, wherein antibody-peptide binding to a peptide having a sequence of SEQ ID NO: 2 is associated with chronological age.

38. The method of any one of claims 9 to 37, wherein the plurality of healthy reference subjects are subjects not having an immune altering condition.

39. The method of claim 38, wherein the immune altering condition is selected from an autoimmune disease, an inflammatory disease, an immunodeficiency disease, and a cancer.

40. The method of any one of claims 9 to 39, wherein the set of peptides predictive of immune health are identified by a method comprising:
(i) providing a same peptide array and contacting a plurality of reference samples from a plurality of reference subjects to the peptide array;
(ii) detecting the binding of antibodies present in each of the reference samples to the peptides on the array to obtain a pattern of binding signals for each of the reference samples, wherein each pattern of binding signals corresponds to one of a range of known measurements of at least one marker of immune health;
(iii) measuring the binding signal associated with each peptide in each of the pattern of binding signals obtained for each of the reference samples;
(iii) determining the correlation of the binding signal for each of the peptides in the plurality of reference samples to the range of measurements of the at least one known marker of immune health; and
(iv) identifying a set of peptides having a combination of binding signals that correlates to the at least one marker of immune health, thereby identifying the set of peptides predictive of immune health.

41. The method of claim 40, wherein range of known measurements is selected for chronological age and body mass index.

42. The method of claim 40, wherein step (iv) comprises using a statistical model selected from Elastic Net regression, SVM, and neural networks.

43. The method of claim 40, wherein the at least one marker of immune health is selected from chronological age, body mass index, at least one cytokine, and a combination thereof.

44. The method of claim 40, wherein the at least one marker of immune health further comprises one or more of the group consisting of an immune protein sequence, a cytokine level, a metabolite level, and a blood cell count.

45. The method of any one of claims 9 to 44, wherein the immune health of the subject corresponds an immunological measurement that is less than, equal to, or greater than the same immunological measurement obtained in the healthy reference subjects having a chronological age corresponding to the immune age of the subject.

46. The method of claim 45, wherein the immune health corresponds to a chronological age that is greater than, equal to, or less than the chronological age of the subject, thereby determining that the immune health of the subject is greater than, equal to, or less than the immune age of healthy reference subjects.

47. The method of claim 45, wherein the immune health corresponds to a BMI that is greater than, equal to, or less than the BMI of the subject, thereby determining that the immune health of the subject is greater than, equal to, or less than the immune health of healthy reference subjects.

48. The method of claim 45, wherein the immune health corresponds to a combination of chronological age and BMI that is greater than, equal to, or less than the chronological age of the subject, thereby determining that the immune health of the subject is greater than, equal to, or less than the immune health of healthy reference subjects.

49. The method of any one of claims 40 to 48, wherein the marker is chronological age, and wherein the set of peptides predictive of immune health comprise at least one of the sequence motifs provided in Table 1.

50. The method of any one of claims 40 to 48, wherein the marker is chronological age, and wherein the set of peptides predictive of immune health comprise at least one of the following sequence motifs: S, SS, SSS, SSSS, ST, TS, TT, or TTT.

51. The method of any one of claims 40 to 48, wherein the marker is BMI, and wherein the set of peptides predictive of immune health comprise at least one of the sequence motifs provided in Table 2.

52. The method of any one of claims 40 to 48, wherein the marker is BMI, and wherein the set of peptides predictive of immune health comprise at least one of the following sequence motifs: S, SS, SSS, SSSS, ST, TS, TT, or TTT.

53. The method of any one of claims 2 to 51, wherein the immune protein is selected from the group consisting of an immunoglobulin and a T cell receptor.

54. The method of any one of claims 2 to 53, wherein the immune protein sequence is determined by sequencing a nucleic acid encoding the immune protein.

55. The method of any one of claims 2 to 54, wherein the metabolite is selected from a fatty acid, an amino acid, a sugar, an enzyme substrate, and combinations thereof.

56. The method of any one of claims 2 to 55, wherein the blood cell is selected from one or more of an erythrocyte, a leukocyte, a neutrophil, an eosinophil, a basophil, a lymphocyte, a T cell, a CD4+ T cell, a CD8+ T cell, a regulatory T cell, a γδ T cell, a natural killer cell, a natural killer T cell, a monocyte, a macrophage, and a platelet.

57. A computer-implemented method of predicting an immune health of a subject comprising:
a) ingesting, by a computer, results of an immunological measurement from a biological sample from the subject, wherein the immunological measurement comprises antibody-peptide binding; and
b) applying, by the computer, a machine learning algorithm to the results of the immunological measurement to predict the immune health of the subject.

58. The method of claim 57, further comprising performing, by the computer, feature selection.

59. The method of claim 58, wherein the feature selection is performed by t-test, correlation, principal component analysis (PCA), or a combination thereof.

60. The method of claim 57, wherein the machine learning algorithm is implemented as: a linear classifier, a neural network, a support vector machine (SVM), an adaptively boosted classifier (AdaBoost), decision tree learning, or a combination thereof.

61. The method of claim 60, wherein the machine learning algorithm is implemented as a linear classifier, and wherein a linear model is learned by elastic net.

62. The method of claim 57, wherein the machine learning algorithm is implemented as ridge regression, lasso regression, regression trees, forward stepwise regression, backward elimination, support vector regression, or a combination thereof.

63. The method of claim 57, further comprising comparing, by the computer, a proxy measure of the immune health of the subject to the predicted immune health of the subject to determine a residual score.

64. The method of claim 63, wherein the proxy measure of the immune health of the subject comprises: chronological age, body mass index (BMI), immune disease or immune disease state, response to treatment in autoimmune disease, response to treatment in immunotherapy, erythrocyte sedimentation rate, antinuclear autoantibodies, rheumatoid factor, fibrinogen, T cell TCR diversity, B cell immunoglobulin diversity, quantification of lymphocytes, quantification of myeloid cells, endogenous steroids, quantification of complement, or a combination thereof.

65. The method of claim 64, wherein the proxy measure of the immune health of the subject comprises a combination of chronological age and body mass index (BMI).

66. The method of claim 57, wherein the predicted immune health is expressed as an immune age.

67. The method of claim 57, further comprising ingesting survey data pertaining to the current or past health of the subject, and wherein the machine learning algorithm is further applied to the survey data.

68. The method of claim 57, further comprising generating, by the computer, a report.

69. The method of claim 68, wherein the report is implemented as a mobile application or a web application.

70. The method of any one of claims 57 to 69, wherein the immunological measurement further comprises one or more of the group consisting of: antibody-peptide binding, an immune protein sequence, a cytokine level, a metabolite level, and a blood cell count.

71. The method of claim 70, wherein the cytokine is selected from TNFα, GM-CSF, MCP-1 (CCL2), MCP-3, IFNα, IFNγ, IL1β, IL2, IL4, IL5, IL6, IL7, IL8, IL10, IL12, IL13, IL17, IL18, IL21, CRP, EGFR, IP10 (CXCL10), Eotaxin (CCL11), MIG, AGP, sTNF-RI, sTNF-RII, sIL2RA, sIL1RA, sIL1RII, sIL6R, CD40L, IL18BP, EGF, VEGF, resistin, leptin, adiponectin, alpha-1-antitrypsin, and free fatty acids.

72. The method of any one of claims 70 to 71, wherein the cytokine is selected from CD40L, EGF, Eotaxin (CCL11), GM-CSF, IFNα, IFNγ, IL-1β, sIL-1RA, sIL-2R, IL-6, IP-10 (CXCL10), MCP-1 (CCL2), TNFα, sTNF-RI, sTNF-RII.

73. The method of any one of claims 70 to 71, wherein the cytokine is selected from Eotaxin (CCL11), sIL-1RA, sIL-2R, sTNF-RI, IP10 (CXCL10), TNFα, IFNα, IFNγ, IL6, sTNF-RII, and IL-1β.

74. The method of any one of claims 70 to 73, wherein cytokine level is measured in a biological fluid.

75. The method of claim 74, wherein the biological fluid is selected from the group consisting of serum, whole blood, dried blood, plasma, saliva, and a combination thereof.

76. The method of any one of claims 70 to 75, wherein the cytokine level is measured in a cytokine assay selected from the group consisting of a bead assay, an aptamer assay, an ELISA assay, and an ELISPOT assay.

77. The method of any of claims 57 to 76, wherein antibody-peptide binding is measured in a peptide array binding assay, wherein the peptide array binding assay comprises
(a) contacting a sample from the subject to a peptide array comprising a plurality of different peptides on distinct features of the array;
(b) detecting the binding of antibodies present in the sample to a set of peptides on the peptide array to obtain a pattern of binding signals, wherein the pattern comprises binding signals each associated with a distinct peptide on the array; and
(c) comparing the pattern of binding signals in the sample to the pattern of binding signals obtained in reference samples, wherein the binding signals obtained from the binding of the sample correspond to a same set of peptides predictive of immune health identified in a plurality of healthy reference subjects,
thereby determining the immune health of the subject.

78. The method of claim 77, wherein the sample is a biological fluid.

79. The method of claim 78, wherein the biological fluid is selected from the group consisting of blood, serum, plasma, saliva, and a combination thereof.

80. The method of claim 79, wherein blood is dried blood.

81. The method of any one of claims 77 to 80, wherein the peptide array is a peptide microarray.

82. The method of any one of claims 77 to 80, wherein the peptide array comprises about 10,000 distinct peptides.

83. The method of any one of claims 77 to 80, wherein the peptide array comprises about 3,000,000 distinct peptides.

84. The method of any one of claims 77 to 83, wherein the peptide array comprises peptides having 20 or fewer amino acids.

85. The method of any one of claims 77 to 83, wherein the peptide array comprises peptides having at least 20 amino acids.

86. The method of any one of claims 77 to 85, wherein the peptide array comprises peptides comprising natural amino acids.

87. The method of any one of claims 77 to 86, wherein the peptide array comprises peptides comprising unnatural amino acids.

88. The method of any one of claims 77 to 87, wherein the peptide array comprises a plurality of peptides characterized by at least one serine motif, threonine motif, serine-threonine motif, or any combination thereof.

89. The method of claim 88, wherein the serine motif comprises S, SS, SSS, or SSSS.

90. The method of claim 88, wherein the threonine motif comprises T or TT.

91. The method of claim 88, wherein the serine-threonine motif comprises TS or ST.

92. The method of any one of claims 88-91, wherein each of the at least one serine motif, threonine motif, or serine-threonine motif is positioned no more than 1, 2, 3, 4, 5 or 6 amino acids from the N-terminus.

93. The method of any one of claims 88-92, wherein the plurality of peptides are acetylated.

94. The method of any one of claims 88 to 93, wherein the machine learning algorithm comprises a panel of peptide features comprising the plurality of peptides characterized by at least one serine motif, threonine motif, serine-threonine motif, or any combination thereof.

95. The method of claim 94, wherein the plurality of peptides make up at least 10%, 20%, 30%, 40%, 50%, 60%, 70%, 80%, 90%, or 95% of the panel of peptide features.

96. The method of claim 94 or 95, wherein the plurality of peptides comprises at least 50, 100, 150, 200, 250, 300, or 350 peptides.

97. The method of claim 94 or 96, wherein at least 10%, 20%, 30%, 40%, 50%, 60%, 70%, 80%, 90%, or 95% of the plurality of peptides are statistically correlated with age.

98. The method of claim 97, wherein at least 10%, 20%, 30%, 40%, 50%, 60%, 70%, 80%, 90%, or 95% the plurality of peptides that are statistically correlated with age have an N-terminal di-serine (SS) motif.

99. The method of any one of claims 77 to 87, wherein the peptide array comprises peptides having a sequence comprising EX₁(X₂)ₙ (SEQ ID NO: 1), wherein X₁ comprises an amino acid selected from A, S, R, Y, and V, X₂ comprises any amino acid.

100. The method of claim 99, wherein n is between 3 and 30.

101. The method of claim 99 or claim 100, wherein antibody-peptide binding to a peptide having a sequence of SEQ ID NO: 1 is associated with body mass index (BMI).

102. The method of any one of claims 77 to 101, wherein the peptide array comprises peptides having a sequence comprising SS(X)ₙ (SEQ ID NO: 2), wherein X comprises any amino acid.

103. The method of claim 102, wherein n is between 3 and 30.

104. The method of claim 102, wherein antibody-peptide binding to a peptide having a sequence of SEQ ID NO: 2 is associated with chronological age.

105. The method of any one of claims 77 to 104, wherein the plurality of healthy reference subjects are subjects not having an immune altering condition.

106. The method of claim 105, wherein the immune altering condition is selected from an autoimmune disease, an inflammatory disease, an immunodeficiency disease, and a cancer.

107. The method of any one of claims 77 to 106, wherein the set of peptides predictive of immune health are identified by a method comprising:
(i) providing a same peptide array and contacting a plurality of reference samples from a plurality of reference subjects to the peptide array;
(ii) detecting the binding of antibodies present in each of the reference samples to the peptides on the array to obtain a pattern of binding signals for each of the reference samples, wherein each pattern of binding signals corresponds to one of a range of known measurements of at least one marker of immune health;
(iii) measuring the binding signal associated with each peptide in each of the pattern of binding signals obtained for each of the reference samples;
(iii) determining the correlation of the binding signal for each of the peptides in the plurality of reference samples to the range of measurements of the at least one known marker; and
(iv) identifying a set of peptides having a combination of binding signals that correlates to the at least one marker of immune health, thereby identifying the set of peptides predictive of immune health.

108. The method of claim 107, wherein range of known measurements is selected for chronological age and body mass index.

109. The method of claim 107, wherein step (iv) comprises using a statistical model selected from Elastic Net regression, SVM, and neural networks.

110. The method of claim 107, wherein the at least one marker of immune health is selected from chronological age, body mass index, at least one cytokine, and a combination thereof.

111. The method of claim 107, wherein the at least one marker of immune health further comprises one or more of the group consisting of an immune protein sequence, a cytokine level, a metabolite level, and a blood cell count.

112. The method of any one of claims 57 to 111, wherein the immune health of the subject corresponds an immunological measurement that is less than, equal to, or greater than the same immunological measurement obtained in the healthy reference subjects having a chronological age corresponding to the immune health of the subject.

113. The method of claim 112, wherein the immune health corresponds to a chronological age that is greater than, equal to, or less than the chronological age of the subject, thereby determining that the immune health of the subject is greater than, equal to, or less than the immune age of healthy reference subjects.

114. The method of claim 112, wherein the immune health corresponds to a BMI that is greater than, equal to, or less than the BMI of the subject, thereby determining that the immune health of the subject is greater than, equal to, or less than the immune health of healthy reference subjects.

115. The method of claim 112, wherein the immune health corresponds to a combination of chronological age and BMI that is greater than, equal to, or less than the chronological age of the subject, thereby determining that the immune health of the subject is greater than, equal to, or less than the immune health of healthy reference subjects.

116. The method of any one of claims 107 to 115, wherein the marker is chronological age, and wherein the set of peptides predictive of immune health comprise at least one of the sequence motifs provided in Table 1.

117. The method of any one of claims 107 to 115, wherein the marker is chronological age, and wherein the set of peptides predictive of immune health comprise at least one of the following sequence motifs: S, SS, SSS, SSSS, ST, TS, TT, or TTT.

118. The method of any one of claims 107 to 115, wherein the marker is BMI, and wherein the set of peptides predictive of immune health comprise at least one of the sequence motifs provided in Table 2.

119. The method of any one of claims 107 to 115, wherein the marker is BMI, and wherein the set of peptides predictive of immune health comprise at least one of the following sequence motifs: S, SS, SSS, SSSS, ST, TS, TT, or TTT.

120. The method of any one of claims 1 to 119, further comprising providing a recommendation for the subject based on the measured immune health.

121. The method of claim 120, wherein the recommendation comprises providing treatment to the subject, stopping treatment of the subject, adopting a lifestyle change, or obtaining testing for one or more immune-related diseases, disorders, or conditions.

122. The method of any one of claims 1 to 119, further comprising providing a therapy or treatment to the subject based on the measured immune health.

123. The method of any one of claims 1 to 119, further comprising providing further testing to the subject based on the measured immune health.

124. The method of claim 123, wherein the further testing comprises genetic testing, metabolite testing, serum protein testing, blood cell count testing, immunoglobulin testing, or any combination thereof.

125. A computer system comprising a processor and non-transitory computer readable storage medium encoded with a computer program that causes the processor to perform the method of any one of claims 57-121.
